(19) United States
(12) Patent Application Publication
(10) Pub. No.: US 2024/0042030 A1
(43) Pub. Date: Feb. 8, 2024

(54) ENGINEERED CELLS WITH REDUCED GENE EXPRESSION TO MITIGATE IMMUNE CELL RECOGNITION

(71) Applicant: Allogene Therapeutics, Inc., South San Francisco, CA (US)

(72) Inventors: Cesar Adolfo SOMMER, San Mateo, CA (US); Hsin-Yuan CHENG, San Mateo, CA (US); Barbra Johnson SASU, San Francisco, CA (US)

(21) Appl. No.: 18/361,056

(22) Filed: Jul. 28, 2023

Related U.S. Application Data
(60) Provisional application No. 63/509,136, filed on Jun. 20, 2023.
(60) Provisional application No. 63/393,350, filed on Jul. 29, 2022.

(51) Int. Cl. A61K 39/00; C12N 15/113; C12N 5/0783; A61P 35/00

(52) U.S. Cl. CPC A61K 39/4631; C12N 15/113; A61K 39/4613; C12N 5/0646; A61P 35/00; C12N 2510/00

(57) ABSTRACT

Provided herein are engineered immune cells and populations thereof for administration to subjects to treat cancer (e.g., solid tumors or liquid tumors) and other conditions. The cells are engineered to functionally express a reduced level of one or more of CD48, CD58, ICAM-1, RFX5, NLRC5, TAP2, β2m, TRAC, RFXAP, CIITA and RFXANK. The cells optionally are further engineered to express one or more than one additional protein such as an antigen binding protein (e.g., a chimeric antigen receptor (CAR) or T cell receptor) and/or a CD70 binding protein to target tumor cells or other damaged cells in the subject and/or to express other genes at a reduced level. Also provided are methods of making and using the engineered cells, compositions and kits comprising them, and methods of treating by administering the cells and the compositions.

### CROSS REFERENCE TO RELATED APPLICATIONS

The present application claims the benefit of priority to U.S. Provisional Application No. 63/393,350, filed on Jul. 29, 2022; and U.S. Provisional Application No. 63/509,136, filed on Jun. 20, 2023, the contents of both of which are hereby incorporated by reference in their entireties.

### REFERENCE TO SEQUENCE LISTING

The instant application contains a Sequence Listing which has been submitted electronically in XML file format and is hereby incorporated by reference in its entirety. Said XML copy, created on Jul. 13, 2023, is named AT-053_03US_SL.xml and is 98,189 bytes in size.

### FIELD

The present disclosure relates generally to the use of engineered immune cells (e.g., T cells) for use in therapeutic applications.

### BACKGROUND

Adoptive transfer of immune cells genetically modified to recognize malignancy-associated antigens is showing promise as a new approach to treating cancer (see, e.g., Brenner et al., Current Opinion in Immunology, 22(2): 251-257 (2010); Rosenberg et al., Nature Reviews Cancer, 8(4): 299-308 (2008)). Immune cells can be genetically modified to express chimeric antigen receptors (CARs), fusion proteins comprised of an antigen recognition moiety and T cell activation domains (see, e.g., Eshhar et al., Proc. Natl. Acad. Sci. USA, 90(2): 720-724 (1993)). Immune cells that contain CARs, e.g., CAR-T cells (CAR-Ts), are engineered to endow them with antigen specificity while retaining or enhancing their ability to recognize and kill a target cell.

However, the generation of CAR-modified autologous cell therapies is expensive, requires weeks of process and quality testing, and yields product of variable potency depending on the initial quality and quantity of patient-specific T cells employed. Allogeneic CAR-modified cell therapies—in which cells from a healthy donor are modified to express the CAR and then administered to multiple patients-promises a cheaper and more robust product than autologous therapies that can be delivered immediately upon need (see, e.g., Graham et al., Cells 2018, 7, 155; doi:10.3390/cells7100155). Additionally, allogeneic therapies enable selection based on desirable product characteristics (e.g., gene editing efficiency, site of integration, lack of deleterious off-target gene edits, haplotype, etc.), and facilitate more sophisticated cell engineering (e.g., multiple gene edits improving potency, persistence, homing, etc.). A major hurdle to implementing allogeneic CAR-modified cell therapies is the potential for rejection of the product (based on donor cells) by the immune system of the patient (host).

Killer lymphocytes such as CD8+ T cells and natural killer (NK) cells identify and kill cancerous, virally infected, and foreign cells (including allogeneic cells) that deviate from self. The central determinants of self vs non-self-discrimination are the major histocompatibility complex (MHC) molecules expressed on the surface of all nucleated cells. Each MHC class I molecule is a non-covalent trimeric complex of a highly multi-allelic MHC class I heavy chain (the most common of which is HLA-A2), an invariant β2 microglobulin (β2m), and a presented peptide proteolytically-derived from an internally expressed protein. Collectively, MHC class I molecules are loaded with peptides representative of the entire protein diversity expressed within the cell, enabling cancerous and infected cells to ‘announce’ their distress to the immune system. CD8+ T cells recognize these non-self peptides with a T cell receptor (TCR) unique to each nascent T cell and initiate killing pursuant to recognition of non-self peptide-MHC (see, e.g., Dembid, Z. et al. Nature 320, 232-238 (1986)). The recognition of non-self by T cells is complemented by the recognition of ‘missing self’ by NK cells: inhibitory receptors on the surface of NK cells enable NK cell-mediated killing in the absence of MHC (see, e.g., K. Karre et al., Nature 319, 675-678 (1986); see FIGS. 1 and 3 of PCT/US2022/14393, which is incorporated herein by reference in its entirety). Experimental ablation of MHC class I presentation—achieved through CRISPR/Cas9-mediated genomic knockout (KO) of the β2m gene encoding the universal β2m component of MHC class I—results in potent NK activation and selective killing of T cells lacking MHC in both autologous and alloreactive contexts (see FIG. 2 of PCT/US2022/14393, which is incorporated herein by reference in its entirety).

While allogeneic cell therapies have advantages over autologous cell therapies, allogeneic cells face potential rejection by host or recipient immune system cells reactive with T and NK epitope determinants on the surface of the allogeneic cell product that are distinct from host. Approaches to avoid rejection of allogeneic therapeutic cells by downregulating or abolishing expression of HLA molecules on the surface of the allogeneic cells have been described (see, e.g., Lanza, R. et al. Engineering universal cells that evade immune detection. Nat. Rev. Immunol. 2019 December; 19(12):723-733, Epub 2019 Aug. 15).

Abrogation of MHC class I molecules, for example by deleting the beta-2 microglobulin gene (β2m), can prevent recognition and rejection by CD8 T cells but the lack of MHC I is a strong signal for NK cell reactivity, which may result in acute rejection by these cells. Downregulation of MHC molecules by knocking down β2m expression or inactivating genes involved in antigen processing and presentation can potentially avoid NK cell reactivity and decrease, but not completely eliminate, rejection by cytotoxic CD8 T cells. These modifications, typically achieved with the use of gene editing tools or shRNA technology, can be combined with exogenous overexpression of NK cell and/or T cell inhibitory proteins (e.g., CD47 or PDL1) that can further decrease the degree of rejection but are nevertheless not entirely effective (Deuse et al. The SIRPα-CD47 immune checkpoint in NK cells. J Exp Med (2021) 218 (3): e20200839; Han et al. Generation of hypoimmunogenic human pluripotent stem cells. PNAS, Apr. 30, 2019, 116 (21) 10441-10446).

The present disclosure provides the advantages of improved allogeneic therapies that provide increased persistence of the administered cells despite the recipients' natural defenses.

### SUMMARY

Provided herein are immune cells that have been engineered e.g., genetically engineered to mitigate rejection by a host or recipient into which or whom the cells have been introduced, methods of mitigating rejection and/or recognition by a host or recipient's immune system e.g., T cells and/or NK cells, compositions and populations comprising the engineered cells and methods of treating a cancer in a patient using the same.

In one aspect, the disclosure provides an engineered immune cell, e.g., a CAR T cell, or a population of engineered immune cells comprising the engineered immune cell that comprises one or more genomic modifications that functionally impair or reduce expression of one or more targets described herein. In one embodiment, the engineered immune cell comprises a genomic modification that functionally impairs or reduces expression of (i) RFX5 and/or NLRC5 and (ii) CD58 relative to a cell without the genomic modification. In another embodiment, the genomic modification comprises a knockdown and/or a knockout of (i) RFX5 and/or NLRC5 and/or (ii) CD58. In one other embodiment, the genomic modification comprises one or more modifications at the gene locus of (i) RFX5 and/or NLRC5 and (ii) CD58. In one embodiment, the genomic modification comprises a deletion or an insertion at the gene locus of (i) RFX5 and/or NLRC5 and (ii) CD58. In other embodiments, the genomic modification is selected from the group consisting of (i) an insertion of one or more nucleotides, (ii) an insertion of a polynucleotide sequence that encodes a protein, (iii) a deletion of one or more nucleotides, and (iv) a substitution of one or more nucleotides. In additional embodiments, the genomic modification was introduced by a gene editing technology selected from TALEN, zinc finger, Cas-CLOVER, and a CRISPR/Cas system.

In one embodiment, the one or more genomic modifications are at the genomic location of one or more genes (corresponding to one or more targets as described herein) or are elsewhere within the genome and not at the location of the one or more genes (corresponding to one or more targets as described herein), such that the modifications functionally impair or reduce expression of the one or more genes (corresponding to one or more targets as described herein).

In one embodiment, the genomic modification comprises an insertion of an RNA interference sequence. In another embodiment, the RNA interference sequence is an shRNA sequence, an siRNA sequence, or a miRNA sequence. In other embodiments, the RNA interference sequence comprises a sequence that is complementary to (i) RFX5 and/or NLRC5 and/or (ii) CD58 gene sequences.

In a further embodiment, the engineered immune cell further comprises a polynucleotide sequence encoding an antigen binding protein and/or a CD70 binding protein. In one embodiment, the antigen binding protein is a chimeric antigen receptor (CAR) or a T cell receptor (TCR). In another embodiment, the cell is further engineered to comprise one or more genomic modifications that functionally impair or reduce expression of one or more of TAP2, β2m, TRAC, CIITA, RFXAP, RFXANK, ICAM-1, and CD48 relative to a non-engineered cell.

In additional embodiments, the engineered immune cell or the population of engineered immune cells comprising the engineered immune cell has improved persistence and/or improved resistance against alloreactive immune cell rejection as compared to an immune cell that does not comprise the genomic modification. In one embodiment, the alloreactive immune cell rejection comprises alloreactive T cell-mediated rejection and/or alloreactive natural killer (NK) cell-medicated rejection. In other embodiments, the increased persistence is determinable and/or determined by a mixed lymphocyte reaction (MLR) assay. In one embodiment, the improved resistance against alloreactive immune cell rejection is determinable and/or determined by an MLR assay.

In other embodiments, the engineered immune cell comprises one or more genomic modifications that functionally impair or reduce expression of RFX5 and CD58. In one embodiment, the engineered immune cell comprises one or more genomic modifications that functionally impair or reduce expression of NLRC5 and CD58. In another embodiment, the engineered immune cell comprises one or more genomic modifications that functionally impair or reduce expression of RFX5, NLRC5, and CD58. In other embodiments, β2m is functionally expressed at a reduced level in the engineered immune cell. In additional embodiments, the engineered immune cell comprises an unmodified β2m gene, and/or wherein β2m is not functionally expressed at a reduced level in the engineered immune cell.

In further embodiments, the engineered immune cell exhibits: (i) a reduced level of expression of an MHC class I protein or complex at the cell surface and/or (ii) a reduced level of expression of an MHC class II protein or complex at the cell surface.

In one embodiment, the antigen binding protein is a CAR. In other embodiments, the engineered immune cell expresses the antigen binding protein and/or the CD70 binding protein. In further embodiments, the polynucleotide sequence encoding the antigen binding protein and/or the CD70 binding protein is located within a disrupted CD58, RFX5, NLRC5, ICAM-1, CD48, TAP2, β2m, TRAC, CIITA, RFXAP or RFXANK locus.

In other embodiments, the engineered immune cell further comprises one or more genomic modifications of an endogenous TCRa gene. In one embodiment, the engineered immune cell further comprises one or more genomic modifications of an endogenous CD52 gene.

In one other embodiment, the engineered immune cell is or is obtained from an immune cell of a healthy volunteer, is obtained from a patient, or is obtained from an induced pluripotent stem cell (iPSC). In other embodiments, the engineered immune is not a natural killer (NK) cell or is not obtained from an NK cell of a healthy volunteer or patient. In a further embodiment, the engineered immune cell is not obtained from an iPSC.

In one embodiment, the engineered immune cell or one or more engineered immune cells in a population of engineered immune cells expresses or functionally expresses a CD70 binding protein. In other embodiments, the engineered immune cell comprises a polynucleotide sequence encoding a CD70 binding protein. In a further embodiment, the CD70 binding protein comprises a CD70 binding domain and a transmembrane domain. In one embodiment, the CD70 binding domain comprises a CD70 antibody, or a receptor for CD70 or a CD70 binding fragment thereof. In other embodiments, the CD70 binding domain comprises an anti-CD70 antibody, optionally the anti-CD70 antibody is a scFv. In another embodiment, the CD70 binding protein further comprises a hinge domain, optionally the hinge domain comprises a CD8 hinge. In one embodiment, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In a further embodiment, the CD70 binding protein comprises a CD3z or a CD3g signaling domain and does not comprise a costimulatory domain. In other embodiments, the CD70 binding protein comprises a 4-1BB signaling domain and does not comprise a CD3z signaling domain. In another embodiment, the CD70 binding protein comprises a 4-1BB signaling domain and a CD3z signaling domain. In other embodiments, the one or more intracellular domain comprises the amino acid sequence of one or more of SEQ ID NOs: 1, 7-14, 17-31, 32-58, 59-70, or 89-90. In another embodiment, the CD70 binding protein does not comprise an intracellular signaling domain.

In one other embodiment, the engineered immune cell or one or more engineered immune cells in a population of engineered immune cells comprise an unmodified β2m, RFX5, NLRC5, CIITA, and TAP2 gene. In another embodiment, the expression of one or more of β2m, RFX5, NLRC5, CIITA, and TAP2 is not functionally impaired or reduced in the engineered immune cell or in one or more engineered immune cells in a population of engineered immune cells. In one embodiment, the one or more genomic modifications do not comprise genomic modifications of one or more of β2m, RFX5, NLRC5, CIITA, and TAP2.

In one aspect, the present disclosure provides a population of engineered immune cells that comprise one or more of the engineered immune cells described herein. In one embodiment, the population is characterized as having no more than 50% of the engineered immune cells functionally express (i) RFX5 and/or NLRC5 and (ii) CD58. In other embodiments, no more than 50% of the engineered immune cells functionally express RFX5 and CD58; or no more than 50% of the engineered immune cells functionally express NLRC5 and CD58; or no more than 50% of the engineered immune cells functionally express RFX5, NLRC5, and CD58. In other embodiments, no more than 50% of the engineered immune cells also functionally express a) any one or more of CD48, ICAM-1, TAP2, β2m, TRAC, CIITA, RFXAP and RFXANK; or b) only one of CD48 and ICAM-1; or c) both CD48 and ICAM-1.

In one embodiment, the population of engineered immune cells comprises engineered immune cells, wherein at least 1% of the engineered immune cells functionally express (i) RFX5 and/or NLRC5 and (ii) CD58 at a level not greater than 50% of the expression level in non-engineered immune cells. In another embodiment, the at least 1% of engineered immune cells functionally express RFX5 and CD58 at a level not greater than 50% of the expression level in non-engineered immune cells. In one embodiment, the at least 1% of engineered immune cells functionally express NLRC5 and CD58 at a level not greater than 50% of the expression level in non-engineered immune cells. In another embodiment, the at least 1% of engineered immune cells functionally express RFX5, NLRC5, and CD58 at a level not greater than 50% of the expression level in non-engineered immune cells. In additional embodiments, the at least 1% of engineered immune cells functionally express a) any one or more of CD48, ICAM-1, TAP2, β2m, TRAC, CIITA, RFXAP and RFXANK; or b) only one of CD48 and ICAM-1; or c) both CD48 and ICAM-1, at a level not greater than 50% of the expression level in non-engineered immune cells.

In a further embodiment, the population comprises and that has improved persistence and/or improved resistance against alloreactive immune cell rejection as compared to a non-engineered immune cell. In another embodiment, the improved resistance is against alloreactive T cell-mediated rejection and/or alloreactive natural killer (NK)-mediated rejection. In one embodiment, the increased persistence is determinable and/or determined by a mixed lymphocyte reaction (MLR) assay and/or wherein the improved resistance against alloreactive immune cell rejection is determinable and/or determined by an MLR assay.

In another embodiment, the population of engineered immune cells comprises engineered immune cells, wherein at least 50% of the engineered immune cells exhibit a reduced level of expression of an MHC class I protein or complex at the cell surface. In another embodiment, the population of engineered immune cells comprises at least 10% engineered T cells, at least 20% engineered T cells, at least 30% engineered T cells, at least 40% engineered T cells, at least 50% engineered T cells, at least 75% engineered T cells, or at least 90% engineered T cells. In another embodiment, at least 10%, at least 20%, at least 30%, at least 40%, at least 50%, at least 75%, or at least 90% of the engineered immune cells further express an antigen binding protein or a CD70 binding protein. In a further embodiment, the antigen binding protein is a CAR or a TCR.

In other embodiments, a nucleic acid encoding the antigen binding protein (e.g., CAR or TCR) and/or the CD70 binding protein is inserted into a disrupted locus of CD58, RFX5, NLRC5, CD48, ICAM-1, TAP2, NLRC5, β2m, TRAC, RFX5, RFXAP, CIITA and RFXANK. In a further embodiment, at least 10%, at least 20%, at least 30%, at least 40%, at least 50%, at least 75%, or at least 90% of the engineered cells further comprises one or more genomic modifications of one or more of an endogenous TCRa gene and an endogenous CD52 gene. In one other embodiment, at least 10%, 20%, 30%, 40%, 50%, 75%, or 90% of the engineered immune cells further express one or more proteins selected from the group consisting of HLA-E, HLA-E single-chain trimer, HLA-G or HLA-G single-chain trimer, UL18 or UL18 single-chain trimer, HLA-A2 and HLA-A2 single-chain trimer.

In other embodiments, the functional expression level of one or more of TAP2, NLRC5, β2m, TRAC, RFX5, RFXAP, CIITA and RFXANK is measured by determining the surface expression level of HLA, beta2 microglobulin (B2M) or both HLA and B2M on the surface of the engineered immune cell or is measured by flow cytometry. In one embodiment, the functional expression level of one or more of CD48, CD58, and ICAM-1 is measured by determining the surface expression level of one or more of CD48, CD58, and ICAM-1 on the surface of the engineered immune cell or is measured by flow cytometry.

In one other aspect, the present disclosure provides methods of making the engineered immune cells or populations of engineered immune cells described herein. In one embodiment, the method comprises the step of modifying the genome of an engineered immune cell. In another embodiment, the method further comprises producing the engineered immune cell that comprises genomic modifications. In a further embodiment, the genome of the engineered immune cell is modified using TALEN, zinc finger, Cas-CLOVER, or a CRISPR/Cas system.

In another aspect, the present disclosure provides pharmaceutical composition comprising the engineered immune cell or cell populations comprising the engineered immune cell. In one embodiment, the pharmaceutical composition further comprises at least one pharmaceutically acceptable carrier or excipient. In another embodiment, the engineered immune cell or one or more engineered immune cells of the cell population (i) further express one or more proteins selected from the group consisting of HLA-E, HLA-E single-chain trimer, HLA-G, HLA-G single-chain trimer, UL18, UL18 single-chain trimer, HLA-A2, HLA-A2 single-chain trimer, and human cytomegalovirus (HCMV) US11, and/or (ii) is/are further engineered to not express or to express at a reduced level any one or more of TAP2, NLRC5, β2m, TRAC, CIITA, RFXANK, RFXAP and RFX5.

In one additional aspect, the present disclosure provides methods of treating a condition or a disorder in a patient. In one embodiment, the method comprises administering to the subject an engineered immune cell, a cell population comprising the same, or a pharmaceutical composition comprising the engineered immune cell or a cell population comprising the same. In a further embodiment, the condition is a solid tumor or a liquid tumor. In other embodiments, the disorder is a cancer, an autoimmune disorder, or an infection, as further described herein.

In another aspect, the present disclosure provides methods of improving (i) persistence or (ii) resistance against an alloreactive immune cell rejection of the engineered immune cells described herein. In one embodiment, the engineered immune cells are allogeneic engineered immune cells. In other embodiments, the method comprises the step of modifying allogeneic immune cells to introduce one or more genomic modifications that functionally impair or reduce expression of (i) RFX5 and/or NLRC5 and (ii) CD58 to provide allogeneic engineered immune cells. In a further embodiment, the method comprises the step of administering the allogeneic engineered immune cells to a subject. In one embodiment, the genomic modifications comprise a knockdown and/or a knockout of (i) RFX5 and/or NLRC5 and (ii) CD58. In other embodiments, the genomic modifications comprise one or more modifications at the gene locus of (i) RFX5 and/or NLRC5 and (ii) CD58. In a further embodiment, the genomic modifications comprise a deletion or an insertion at the gene locus of (i) RFX5 and/or NLRC5 and (ii) CD58. In other embodiments, the genomic modifications are selected from the group consisting of (i) an insertion of one or more nucleotides, (ii) an insertion of a sequence that encodes a protein, (iii) a deletion of one or more nucleotides, and (iv) a substitution of one or more nucleotides. In one embodiment, the genomic modifications were introduced by a gene editing technology selected from TALEN, zinc fingers, Cas-CLOVER, and a CRISPR/Cas system.

In an additional embodiment, the genomic modifications comprise an insertion of an RNA interference sequence. In a further embodiment, the interference sequence is an shRNA sequence, an siRNA sequence, or a miRNA sequence. In other embodiments, the interference sequence comprises a sequence that is complementary to (i) RFX5 and/or NLRC5 and (ii) CD58 gene sequences. In some embodiments, the allogeneic engineered immune cells further comprise a polynucleotide sequence encoding an antigen binding protein (e.g., a CAR or a TCR) and/or a CD70 binding protein. In further embodiments, the allogeneic engineered immune cells are further engineered to comprise one or more genomic modifications that functionally impair or reduce expression of one or more of TAP2, β2m, TRAC, CIITA, RFXAP, RFXANK, ICAM-1, and CD48 relative to cells without the modification. In other embodiments, the genomic modifications functionally impair or reduce expression to about 50% or less of the corresponding level in cells without the genomic modifications. In one embodiment, the allogeneic engineered immune cells have improved persistence and/or improved resistance against an alloreactive immune cell rejection as compared to allogeneic non-engineered immune cells. In other embodiments, the improved resistance is against alloreactive T cell-mediated rejection and/or alloreactive natural killer (NK)-mediated rejection. In one embodiment, the increased persistence is determinable and/or determined by a mixed lymphocyte reaction (MLR) assay and/or wherein the improved resistance against alloreactive immune cell rejection is determinable and/or determined by an MLR assay.

In a further embodiment, the method comprises functionally impairing or reducing expression of RFX5 and CD58, or NLRC5 and CD58, or RFX5, NLRC5, and CD58. In an additional embodiment, the extent of reduction in the expression level of (i) RFX5 and/or NLRC5 and (ii) CD58 is determined relative to the expression level of (i) RFX5 and/or NLRC5 and (ii) CD58, respectively, in a cell of the same type that has not been modified.

In a further embodiment, the functional expression level of one or more of CD48, CD58, and ICAM-1 is measured by determining the surface expression level of a CD48 protein, a CD58 protein or an ICAM-1 protein on the surface of the engineered immune cell. In another embodiment, the functional expression level of RFX5 and/or NLRC5 is measured by determining the surface expression level of HLA, beta2 microglobulin (B2M) or both HLA and B2M on the surface of the engineered immune cell. In one embodiment, the surface expression level is measured by flow cytometry. In other embodiments, the method further comprises introducing one or more genomic modifications of one or more of a TCRa gene and a CD52 gene. In one embodiment, the allogeneic engineered immune cells comprise an unmodified β2m gene or wherein β2m is not functionally expressed at a reduced level in the allogeneic engineered immune cell.

In other embodiments, the immune cells of the instant disclosure are engineered to functionally express a reduced level, relative to corresponding cells that have not been so engineered, of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In an additional embodiment, the immune cells that are engineered to functionally express at a reduced level, relative to corresponding cells that have not been so engineered, one or more targets as described herein, comprise one or more genomic modifications that functionally impair or reduce expression of the one or more targets relative to a cell without the one or more genomic modifications.

In another embodiment, the engineered cells (i) have an unmodified β2m gene, (ii) functionally express a normal level of β2m and/or (iii) are not engineered to functionally express a reduced level of β2m.

The engineered cells can be further engineered to augment resistance to rejection and/or to provide a therapeutic effect, e.g., the cells can be engineered to comprise or express an additional protein, e.g., an antigen binding protein such as a chimeric antigen receptor (CAR) and/or a T cell receptor, wherein the antigen binding protein or T cell receptor targets the engineered immune cell to tumor cells that express a cognate antigen and/or to other undesired e.g., disease state cells.

The present disclosure thus provides a method of increasing persistence of allogeneic cells in a recipient, the method comprising engineering the cells to functionally express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   relative to non-engineered cells.

In an additional embodiment, the method of engineering the cells to functionally express at a reduced level, relative to corresponding cells that have not been so engineered, one or more targets as described herein, comprises introducing into the cells one or more genomic modifications that functionally impair or reduce expression of the one or more targets relative to a cell without the one or more genomic modifications.

In another embodiment, the method does not comprise engineering the cells to functionally express a reduced level of β2m.

In an aspect, the present disclosure provides an engineered immune cell that functionally expresses
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level relative to non-engineered cells.

In an additional embodiment, the engineered immune cell that functionally expresses at a reduced level, relative to corresponding cells that have not been so engineered, one or more targets as described herein, comprises one or more genomic modifications that functionally impairs or reduces expression of the one or more targets relative to a cell without the one or more genomic modifications.

In another embodiment, the engineered cell (i) has an unmodified β2m gene, (ii) functionally expresses a normal level of β2m and/or (iii) is not engineered to functionally express a reduced level of β2m. In some embodiments, the reduced level of expression, stated relative to the expression level in a corresponding but non-engineered immune cell, is 0%, for example when both chromosomal copies of a gene are knocked out, or 50% (i.e. 50% of the level in a non-engineered control immune cell), for example when one of the two chromosomal copies of a gene is knocked out and there is no compensatory increase in expression of the other chromosomal copy of that gene. In some embodiments, the cell expresses
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a level not greater than 90%, not greater than 75%, not greater than 50%, not greater than 25%, or not greater than 10% of the expression level in a non-engineered immune cell.

In an additional embodiment, the cell that expresses one or more targets at a level not greater than 90%, not greater than 75%, not greater than 50%, not greater than 25%, or not greater than 10% of the expression level in a non-engineered immune cell comprises one or more genomic modifications that functionally impairs or reduces expression of the one or more targets as described herein relative to a cell without the one or more genomic modifications.

In another embodiment, the cell (i) has an unmodified β2m gene, (ii) functionally expresses a normal level of β2m and/or (iii) is not engineered to functionally express a reduced level of β2m.

In some embodiments, the level of expression of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,

in the engineered immune cell is any value between 0% and 90% of the level in a control cell not correspondingly engineered with respect to the corresponding gene(s). In some embodiments, the expression level in the engineered cell is, for example, between 10% and 90%, between 25% and 90%, between 25% and 75%, between 10% and 50%, between 25% and 50%, between 50% and 90%, or between 50% and 75% of the level in a control cell. In some embodiments, a reduced level of expression other than 0% or 50% is obtained when, for example, only one chromosomal copy of a gene is knocked out and a compensatory mechanism causes an increase in the level of expression of the remaining chromosomal copy, or reduction in expression is achieved by a method other than gene knockout, such as known knockdown methods e.g., those that employ any of various RNA-based techniques (e.g., antisense RNA, miRNA, siRNA; see, e.g., Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016)). In an additional embodiment, the engineered immune cell having a level of expression of one or more targets as described herein that is between 0% and 90% of the level in a control cell that has not been correspondingly engineered comprises comprise one or more genomic modifications that functionally impair or reduce expression of the one or more targets relative to an immune cell without the one or more genomic modifications.

In some embodiments, the engineered immune cell disclosed herein exhibits a reduced level of expression of an MHC class I protein or complex at the cell surface relative to a suitable control. In various embodiments, the cell is a T cell e.g., a human T cell. In some embodiments, the cell comprises a mutation in the TAP2, NLRC5, β2m, CIITA, RFX5, RFXAP and RFXANK loci or gene(s) and/or a disruption in the TAP2, NLRC5, β2m, CIITA, RFX5, RFXAP and RFXANK loci or genes that causes a reduction in functional expression of the disrupted locus (or loci) or gene(s). In other embodiments, the cell may comprise an additional mutation in one or more of the CD48, CD58, and ICAM-1 loci or gene(s) and/or a disruption in one or more of the CD48, CD58, and ICAM-1 loci or gene(s) that causes a reduction in functional expression of the disrupted locus (or loci) or gene(s). In another embodiment, the cell may comprise mutations in
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; or (vi) CD48, CD58, ICAM-1, RFX5, and NLRC5,
   loci or genes that causes a reduction in functional expression of the disrupted locus (or loci) or gene(s). In another embodiment, the cell (i) does not comprise a mutation in the β2m locus or gene, (ii) has an unmodified β2m gene, (iii) functionally expresses a normal level of β2m and/or (iv) is not engineered to functionally express a reduced level of β2m.

In an additional embodiment, the cell that comprises mutations in the loci or genes of one or more targets as described herein that cause a reduction in functional expression of the disrupted locus (or loci) or gene(s) comprise one or more genomic modifications that functionally impair or reduce expression of the one or more targets relative to a cell without the one or more genomic modifications.

In an embodiment, the mutation or disruption is introduced by any one or a combination of gene mutations or gene editing techniques, including but not limited to known homologous recombination techniques and techniques that employ any one or more of meganucleases, TALEN, zinc fingers, shRNA, Cas-CLOVER, and a CRISPR/Cas system (for example, Cas9, Cas12 and MAD7) or other systems such as a base editing system or a prime editing system. In some embodiments, the cell is a non-human cell, e.g., a primate cell or a non-primate mammalian cell. In some embodiments, the cell is a human cell.

In various embodiments, the engineered immune cell further expresses an antigen binding protein, for example, the engineered immune cell comprises a nucleic acid that encodes an antigen binding protein. In an embodiment, the antigen binding protein is a chimeric antigen receptor (CAR). In an embodiment, a nucleic acid encoding the antigen binding protein e.g., the CAR is inserted into a disrupted CD48, CD58, ICAM-1, TAP2, NLRC5, β2m, TRAC, CIITA, RFX5, RFXAP or RFXANK locus or is inserted into the locus, thereby disrupting it. In various embodiments, the antigen binding protein is a T cell receptor (TCR). In an embodiment, a nucleic acid encoding the TCR is inserted into a disrupted CD48, CD58, ICAM-1, TAP2, NLRC5, β2m, TRAC, CIITA, RFX5, RFXAP or RFXANK locus or is inserted into the locus, thereby disrupting it. In an embodiment, the engineered immune cell further comprises one or more genomic modifications, e.g., a modification of an endogenous genetic locus, for example, of one or more of the following: an endogenous CD70 gene, an endogenous TCRa gene and an endogenous CD52 gene. In various embodiments, the one or more genomic modifications cause a reduction or absence of functional expression of the gene that contains the modification.

In an additional embodiment, the engineered immune cell that further expresses an antigen binding protein, e.g., a CAR, comprises one or more genomic modifications that functionally impair or reduce expression of the one or more targets relative to an immune cell without the one or more genomic modifications.

The engineered immune cell can be derived from cells from any of various sources. The engineered immune cell can be prepared or derived from cells e.g., stem cells or immune cells from a person other than the person to whom the engineered immune cells will be administered, e.g., a donor (e.g., a healthy volunteer) other than the recipient, or can be prepared or derived from cells e.g., stem cells or immune cells from the person to whom the engineered immune cells will be administered (the recipient), or can be derived from one or more induced pluripotent stem cells (iPSCs). In an embodiment, the immune cell is an immune cell obtained from a healthy volunteer, is obtained from a patient, or is derived from an iPSC.

In another aspect, the engineered cells may further comprise a polynucleotide that encodes a CD70 binding protein and/or may functionally express a CD70 binding protein. In one embodiment, the CD70 binding protein comprises a CD70 binding domain and a transmembrane domain. In some embodiments, the CD70 binding domain comprises a CD70 antibody, or a receptor for CD70 or a CD70 binding fragment thereof. In other embodiments, the CD70 binding domain comprises an anti-CD70 antibody, optionally the anti-CD70 antibody is a scFv. In one embodiment, the CD70 binding protein further comprises a hinge domain, optionally the hinge domain comprises a CD8 hinge. In one other embodiment, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In another embodiment, the CD70 binding protein comprises a CD3z or a CD3g signaling domain and does not comprise a costimulatory domain. In additional embodiments, the CD70 binding protein comprises a 4-1BB signaling domain and does not comprise a CD3z signaling domain. In other embodiments, the CD70 binding protein comprises a 4-1BB signaling domain and a CD3z signaling domain. In one embodiment, the one or more intracellular domain comprises the amino acid sequence of one or more of SEQ ID NOs: 1, 7-14, 17-70, or 89-90. In another embodiment, the CD70 binding protein does not comprise an intracellular signaling domain.

In another aspect, the present disclosure provides a method of making the engineered immune cell disclosed herein. In an embodiment, the method comprises the use of any gene editing technology, such as TALEN, zinc fingers, Cas-CLOVER, and a CRISPR/Cas system, and/or the use of any known gene knockdown methods e.g., those that employ any of various RNA-based techniques (e.g., shRNA, antisense RNA, miRNA, siRNA; see, e.g., Lam et al., Mol. Ther. Nucleic Acids 4:e252 (2015), doi:10.1038/mtna.2015.23; Sridharan and Gogtay, Brit. J. Clin. Pharmacol. 82: 659-72 (2016)) to reduce functional expression of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   relative to non-engineered immune cells. In another embodiment, method does not comprise gene editing of the cells to functionally express a reduced level of β2m.

In an additional embodiment, the method of making the engineered immune cell using a gene editing technology to reduce the functional expression, relative to corresponding immune cells that have not been so engineered, of one or more targets as described herein comprises introducing into the immune cell one or more genomic modifications that functionally impair or reduce expression of the one or more targets relative to an immune cell without the one or more genomic modifications.

In an embodiment, the method comprises or further comprises the introduction into the engineered immune cell of a nucleic acid encoding an antigen binding protein, e.g., a CAR or TCR. In an embodiment, the method comprises or further comprises introducing into the genome of the engineered immune cell one or more genomic modifications of one or more of an endogenous TCRa gene and an endogenous CD52 gene. In an embodiment, the one or more genomic modifications disrupts and/or prevents, wholly or partly, the functional expression of one or more of an endogenous TCRa gene and an endogenous CD52 gene.

In various embodiments of the present disclosure, the functional expression level of any one or more of TAP2, NLRC5, β2m, TRAC, CIITA, RFX5, RFXAP and RFXANK is measured by determining the surface expression level of one or more HLA proteins, such as an HLA-A or HLA-B protein, or of beta2 microglobulin (B2M), or of both one or more HLA proteins and B2M on the surface of the engineered immune cell, or is measured by flow cytometry. In various embodiments of the present disclosure, the functional expression level of any one or more of CD48, CD58, and ICAM-1 is measured by determining the surface expression level of each cell surface protein, such as one or more of a CD48, a CD58 or an ICAM-1 protein on the surface of the engineered immune cell, or is measured by flow cytometry. In some embodiments, expression of any one or more of CD48, CD58, ICAM-1, TAP2, NLRC5, β2m, TRAC, CIITA, RFX5, RFXAP and RFXANK in engineered immune cells of the present disclosure is assayed by measuring the degree to which the engineered immune cells survive in the presence of effector cells e.g., T cells, in comparison to the degree to which not correspondingly engineered, but otherwise comparable e.g., identical, immune cells survive under the same conditions.

In some embodiments, an immune cell that has been engineered to functionally express a reduced level, relative to corresponding cells that have not been so engineered, of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   is further engineered (a) to express one or more proteins selected from the group consisting of HLA-E, HLA-E single-chain trimer, HLA-G, HLA-G single-chain trimer, UL18, UL18 single-chain trimer, HLA-A2, HLA-A2 single-chain trimer, human cytomegalovirus (HCMV) US11 and/or (b) to express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   achieved by knockout or knockdown as described herein.

In an additional embodiment, the immune cell that has been engineered to functionally express at a reduced level, relative to corresponding cells that have not been so engineered, one or more targets as described herein, comprises one or more genomic modifications that functionally impair or reduce expression of the one or more targets relative to a cell without the one or more genomic modifications.

In another aspect, the present disclosure provides a population of engineered immune cells comprising an engineered immune cell provided herein. In an embodiment, the population of engineered immune cells comprises between 10⁴ and 10¹⁰ engineered immune cells provided herein. In various embodiments, the population of engineered immune cells comprises 10⁴, 10⁵, 10⁶, 10⁷, 10⁸, 10⁹, or 10¹⁰ engineered immune cells provided herein.

In another aspect, the present disclosure provides a population of engineered immune cells wherein no more than, for example, 75% of the cells functionally express
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In another embodiment, the cells of the population functionally express β2m at a normal level or do not functionally express β2m at a reduced level.

In an additional embodiment, the population of engineered immune cells comprising between 10⁴ and 10¹⁰ engineered immune cells comprises engineered immune cells that comprise one or more genomic modifications that functionally impair or reduce expression of the one or more targets relative to an immune cell without the one or more genomic modifications.

In another aspect, the present disclosure provides a population of engineered immune cells wherein at least 1%, 5%, 10%, 20%, 30%, 40%, 50%, 75%, 90%, 95% or 100% of the engineered immune cells functionally express
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level. The reduced level may be at a level not greater than 90%, not greater than 75%, not greater than 50%, not greater than 25%, or not greater than 10% of the expression level in a non-engineered immune cell. In particular, the reduced levels may be at a level not greater than 50% of the expression level in non-engineered immune cells. In another embodiment, the population of engineered immune cells functionally express β2m at a normal level or do not functionally express β2m at a reduced level.

In an additional embodiment, the population of engineered immune cells wherein at least 1%, 5%, 10%, 20%, 30%, 40%, 50%, 75%, 90%, 95% or 100% of the engineered immune cells functionally express one or more targets as described herein at a reduced level comprise engineered immune cells that comprise one or more genomic modifications that functionally impair or reduce expression of the one or more targets relative to an immune cell without the one or more genomic modifications.

In some embodiments, the reduced level of expression, stated relative to an appropriate control e.g., the expression level in corresponding immune cells not correspondingly engineered, is 0%, for example when both chromosomal copies of a gene are knocked out (e.g., by methods that employ TALEN, zinc fingers, Cas-CLOVER, and/or CRISPR/Cas system), or 50%, for example when one of the two chromosomal copies of a gene is knocked out and there is no compensatory increase in expression of the other chromosomal copy of that gene. In some embodiments, the reduced level of expression, stated relative to the expression level in non-engineered immune cells, is between 0% and 90% or between any two values intermediate between 0% and 90%, for example between 10% and 90%, between 25% and 90%, between 25% and 75%, between 10% and 50%, between 25% and 50%, between 50% and 90%, and between 50% and 75%. Values within one or more of such intermediate ranges can be obtained when, for example, only one chromosomal copy of a gene is knocked out and a compensatory mechanism causes an increase in the level of expression of the remaining chromosomal copy, or reduction in expression is achieved by a method other than gene knockout, such as known knockdown methods, e.g., those that employ any of various RNA-based techniques (e.g., shRNA anti-sense RNA, miRNA, siRNA; see, e.g., Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016)). In some embodiments of the population of engineered immune cells disclosed herein, some or all of the engineered cells, e.g., 5-10%, 10-25%, 25-50%, 50-90%, or 90-100% exhibit a reduced level of expression of an MHC class I protein and/or MHC class II protein or complex at the cell surface relative to a suitable control.

In various embodiments, the population of engineered immune cells or a population of immune cells comprising engineered immune cells as disclosed herein comprises at least 10% engineered T cells, at least 20% engineered T cells, at least 30% engineered T cells, at least 40% engineered T cells, at least 50% engineered T cells, at least 75% engineered T cells, at least 90% engineered T cells or 100% engineered T cells. Also provided herein is a population of cells of which at least 10%, at least 20%, at least 30%, at least 40%, at least 50%, at least 75%, at least 90% or 100% are engineered immune cells e.g., engineered T cells as disclosed herein.

In some embodiments, at least 10%, at least 20%, at least 30%, at least 40%, at least 50%, at least 75%, or at least 90% of the engineered cells of the population further express an antigen binding protein. In some embodiments, the antigen binding protein is a chimeric antigen receptor (CAR). In some embodiments, a nucleic acid encoding the CAR is inserted into a disrupted CD48, CD58, ICAM-1, TAP2, NLRC5, β2m, TRAC, CIITA, RFX5, RFXAP or RFXANK locus and/or such an insertion disrupts the locus. In some embodiments, the antigen binding protein is a T cell receptor (TCR). In some embodiments, a nucleic acid encoding the TCR is inserted into a disrupted CD48, CD58, ICAM-1, TAP2, NLRC5, β2m, TRAC, CIITA, RFX5, RFXAP or RFXANK locus and/or such an insertion disrupts the locus.

In certain embodiments of the population of engineered immune cells disclosed herein, at least 10%, at least 20%, at least 30%, at least 40%, at least 50%, at least 75%, or at least 90% of the engineered cells further comprises one or more genomic modifications of one or more of an endogenous TCRa gene and an endogenous CD52 gene.

In an embodiment, the population of engineered immune cells is derived from one or more immune cells obtained from a person, for example, from a person other than the person to whom they will be administered, e.g., obtained from a donor other than the recipient or from a healthy volunteer, or is derived from one or more immune cells obtained from a patient e.g., the person to whom they will be administered, or is derived from one or more iPSCs.

In another aspect, the population of engineered cells may further comprise a polynucleotide that encodes a CD70 binding protein and/or may functionally express a CD70 binding protein. In one embodiment, the CD70 binding protein comprises a CD70 binding domain and a transmembrane domain. In some embodiments, the CD70 binding domain comprises a CD70 antibody, or a receptor for CD70 or a CD70 binding fragment thereof. In other embodiments, the CD70 binding domain comprises an anti-CD70 antibody, optionally the anti-CD70 antibody is a scFv. In one embodiment, the CD70 binding protein further comprises a hinge domain, optionally the hinge domain comprises a CD8 hinge. In one other embodiment, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In another embodiment, the CD70 binding protein comprises a CD3z or a CD3g signaling domain and does not comprise a costimulatory domain. In additional embodiments, the CD70 binding protein comprises a 4-1BB signaling domain and does not comprise a CD3z signaling domain. In other embodiments, the CD70 binding protein comprises a 4-1BB signaling domain and a CD3z signaling domain. In one embodiment, the one or more intracellular domain comprises the amino acid sequence of one or more of SEQ ID NOs: 1, 7-14, 17-70, or 89-90. In another embodiment, the CD70 binding protein does not comprise an intracellular signaling domain.

In another aspect, the present disclosure provides a method of making the population of engineered immune cells described herein, wherein the method comprises the use of a gene editing technology, for example, a gene editing technology selected from the group consisting of TALEN, zinc fingers, Cas-CLOVER, and a CRISPR/Cas system, to reduce functional expression of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.
   In one embodiment, the method does not comprise the use of a gene editing technology, for example, a gene editing technology to reduce functional expression of β2m.

In an additional embodiment, the method of making the population of engineered immune cells as described herein using a gene editing technology to reduce the functional expression, relative to corresponding immune cells that have not been so engineered, of one or more targets as described herein comprises introducing into immune cells one or more genomic modifications that functionally impair or reduce expression of the one or more targets relative to immune cells without the one or more genomic modifications.

In another aspect, the present disclosure provides a method of determining or measuring the functional expression level of TAP2, NLRC5, β2m, TRAC, CIITA, RFX5, RFXAP and/or RFXANK in the cells of the population of engineered immune cells disclosed herein, wherein the functional expression level is measured by determining the surface expression level of an HLA protein, beta2 microglobulin (B2M) or both an HLA protein and B2M on the surface of the engineered immune cells, and/or is measured by flow cytometry. In various embodiments of the present disclosure, the functional expression level of any one or more of CD48, CD58, and ICAM-1 in the cells of the population of engineered immune cells, wherein the functional expression level is measured by determining the surface expression level of each cell surface protein, such as one or more of a CD48, a CD58 or an ICAM-1 protein on the surface of the engineered immune cell, or is measured by flow cytometry.

In various embodiments of the engineered immune cell described herein and of the population of engineered immune cells disclosed herein, the engineered immune cell is, or one or more of the engineered immune cells of the population, e.g., at least 10%, 20%, 30%, 40%, 50%, 75%, 90% or 100% of the engineered immune cells of the population, are further engineered (e.g., by any of the methods disclosed herein or by any other method known to the person of ordinary skill in the art) to express one or more proteins selected from the group consisting of HLA-E, HLA-E single-chain trimer, HLA-G, HLA-G single-chain trimer, UL18, UL18 single-chain trimer, HLA-A2, HLA-A2 single-chain trimer, and human cytomegalovirus (HCMV) US11 by any method described herein or by any other method known to the person of ordinary skill in the art.

In another aspect, method of treating a condition in a patient comprises administering to the patient: an engineered immune cell, a population of engineered immune cells, or a pharmaceutical composition comprising an engineered cell or a population of engineered immune cells, wherein the engineered cell and/or an engineered cell of the population further comprises a polynucleotide that encodes a CD70 binding protein and/or functionally expresses a CD70 binding protein. In one embodiment, the CD70 binding protein comprises a CD70 binding domain and a transmembrane domain. In some embodiments, the CD70 binding domain comprises a CD70 antibody, or a receptor for CD70 or a CD70 binding fragment thereof. In other embodiments, the CD70 binding domain comprises an anti-CD70 antibody, optionally the anti-CD70 antibody is a scFv. In one embodiment, the CD70 binding protein further comprises a hinge domain, optionally the hinge domain comprises a CD8 hinge. In one other embodiment, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In another embodiment, the CD70 binding protein comprises a CD3z or a CD3g signaling domain and does not comprise a costimulatory domain. In additional embodiments, the CD70 binding protein comprises a 4-1BB signaling domain and does not comprise a CD3z signaling domain. In other embodiments, the CD70 binding protein comprises a 4-1BB signaling domain and a CD3z signaling domain. In one embodiment, the one or more intracellular domain comprises the amino acid sequence of one or more of SEQ ID NOs: 1, 7-14, 17-70, or 89-90. In another embodiment, the CD70 binding protein does not comprise an intracellular signaling domain.

In another aspect, the present disclosure provides a pharmaceutical composition comprising an engineered immune cell as disclosed herein, wherein the composition further comprises one or more pharmaceutically acceptable carrier or excipient. In another aspect, the present disclosure provides a pharmaceutical composition comprising a population of engineered immune cells as disclosed herein, wherein the composition further comprises one or more pharmaceutically acceptable carrier or excipient. In various embodiments of the composition, the engineered immune cell or one or more of the engineered immune cells of the population, e.g., at least 10%, 20%, 30%, 40%, 50%, 75%, 90% or 100% of the engineered immune cells of the population, express(es) one or more proteins selected from the group consisting of HLA-E, HLA-E single-chain trimer, HLA-G, HLA-G single-chain trimer, UL18, UL18 single-chain trimer, HLA-A2, HLA-A2 single-chain trimer, and human cytomegalovirus (HCMV) US11, and/or do(es) not express or express(es) at a reduced level any one or more of CIITA, RFXANK, RFXAP and RFX5 achieved by knockout or knockdown as described herein.

In another aspect, the engineered cell(s) of the pharmaceutical composition further comprise a polynucleotide that encodes a CD70 binding protein and/or functionally express a CD70 binding protein. In one embodiment, the CD70 binding protein comprises a CD70 binding domain and a transmembrane domain. In some embodiments, the CD70 binding domain comprises a CD70 antibody, or a receptor for CD70 or a CD70 binding fragment thereof. In other embodiments, the CD70 binding domain comprises an anti-CD70 antibody, optionally the anti-CD70 antibody is a scFv. In one embodiment, the CD70 binding protein further comprises a hinge domain, optionally the hinge domain comprises a CD8 hinge. In one other embodiment, the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof. In another embodiment, the CD70 binding protein comprises a CD3z or a CD3g signaling domain and does not comprise a costimulatory domain. In additional embodiments, the CD70 binding protein comprises a 4-1BB signaling domain and does not comprise a CD3z signaling domain. In other embodiments, the CD70 binding protein comprises a 4-1BB signaling domain and a CD3z signaling domain. In one embodiment, the one or more intracellular domain comprises the amino acid sequence of one or more of SEQ ID NOs: 1, 7-14, 17-70, or 89-90. In another embodiment, the CD70 binding protein does not comprise an intracellular signaling domain.

In an additional embodiment, the pharmaceutical compositions comprise one or more pharmaceutically acceptable carriers or excipients and an engineered cell, e.g., an engineered immune cell, as described herein, wherein the engineered cell comprises one or more genomic modifications that functionally impair or reduce expression of one or more targets as described herein relative to cells without the one or more genomic modifications.

In an aspect, there is provided an engineered immune cell as disclosed herein, a population of engineered immune cells as disclosed herein, or a pharmaceutical composition for use as a medicament.

In another aspect, the present disclosure provides a method of treating a condition in a patient comprising administering to the patient an engineered immune cell as disclosed herein, a population of engineered immune cells as disclosed herein, or a pharmaceutical composition as disclosed herein. In an embodiment, the condition is selected from the group consisting of a solid tumor and a liquid tumor.

In another aspect, the present disclosure provides a method of decreasing the surface expression level of an MHC class I protein in an engineered immune cell, in some embodiments, to about 75% or less of the expression level of the MHC class I protein in non-engineered immune cells, the method comprising reducing the functional expression level of TAP2, NLRC5, β2m, CIITA, RFX5, RFXAP and RFXANK, e.g., to about 75% or less of the expression level in non-engineered immune cells. In another embodiment, the method further comprises reducing the functional expression level of one or more of CD48, CD58, and ICAM-1, e.g., to about 75% or less of the expression level in non-engineered immune cells. In another embodiment, the method comprises reducing the functional expression level of
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; or (vi) CD48, CD58, ICAM-1, RFX5, and NLRC5,
   e.g., to about 75% or less of the expression level in non-engineered immune cells.

In an additional embodiment, the method of decreasing the surface expression level of an MHC class I protein in an engineered immune cell, in some embodiments, to about 75% or less of the expression level of the MHC class I protein in non-engineered immune cells by reducing the functional expression level of one or more targets as described herein comprises introducing into immune cells one or more genomic modifications that functionally impair or reduce expression of the one or more targets relative to immune cells without the one or more genomic modifications.

In another embodiment, the engineered immune cell functionally express β2m at a normal level or does not functionally express β2m at a reduced level. In another embodiment, the engineered immune cell further comprises a polynucleotide that encodes a CD70 binding protein and/or functionally expresses a CD70 binding protein, as described herein.

In an embodiment, the engineered immune cell disclosed herein exhibits a reduced level of expression of an MHC class I protein or complex at the cell surface relative to a suitable control.

In an embodiment, the engineered immune cell disclosed herein is an engineered T cell. In certain embodiments, the engineered immune cell disclosed herein, e.g., the engineered T cell disclosed herein, further expresses an additional protein e.g., a protein encoded by exogenous DNA or a protein whose expression is brought about by further engineering of the cell. In some embodiments, the additional protein is an antigen binding protein and/or a CD70 binding protein. In some embodiments, the antigen binding protein is a chimeric antigen receptor (CAR). In some embodiments, a nucleic acid encoding the additional protein(s), e.g., the antigen binding protein e.g., the CAR, and/or CD70 binding protein is introduced into the cell by methods described herein. In some embodiments, the nucleic acid is introduced into a disrupted CD48, CD58, ICAM-1, TAP2, NLRC5, β2m, TRAC, CIITA, RFX5, RFXAP or RFXANK locus or a CD48, CD58, ICAM-1, TAP2, NLRC5, β2m, TRAC, CIITA, RFX5, RFXAP or RFXANK locus is disrupted by insertion of a nucleic acid encoding the additional protein e.g., the antigen binding protein, e.g., the CAR, and/or the CD70 binding protein.

In an embodiment of the method disclosed herein, the antigen binding protein is a T cell receptor (TCR) component, e.g., TCR α (TCR alpha), TCR R (TCR beta), TCR 7 (TCR gamma) or TCR δ (TCR delta). In an embodiment, a nucleic acid encoding the TCR component is inserted into a disrupted CD48, CD58, ICAM-1, TAP2, NLRC5, β2m, TRAC, CIITA, RFX5, RFXAP or RFXANK locus or a CD48, CD58, ICAM-1, TAP2, NLRC5, β2m, TRAC, CIITA, RFX5, RFXAP or RFXANK locus is disrupted by insertion of a nucleic acid encoding the TCR component.

In a further embodiment of the method disclosed herein, the engineered immune cell further comprises one or more genomic modifications (e.g., knock-out, deletion, knock-down, insertion) of one or more of an endogenous TCR alpha gene and an endogenous CD52 gene. In some embodiments, the genomic modification partially or wholly eliminates functional expression of the modified gene. In a further embodiment of the method disclosed herein, the immune cell is an immune cell obtained from a person, e.g., from a donor other than the person to whom the cells will be administered, e.g., from a healthy volunteer, or is obtained from a patient e.g., the person to whom the cells will be administered, or is derived from an iPSC.

In another aspect, the present disclosure provides a method of making the population of engineered immune cells disclosed herein, wherein the method comprises the use of a gene editing technology, for example a gene editing technology selected from the group consisting of TALEN, zinc fingers, shRNA, Cas-CLOVER, and a CRISPR/Cas system, to reduce functional expression of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   in a cell, e.g., in an immune cell.

In another embodiment, the immune cell (before or after gene-editing) further comprises a polynucleotide that encodes a CD70 binding protein and/or functionally expresses a CD70 binding protein, as described herein. In another embodiment, the method does not comprise gene editing of β2m in a cell, e.g., in an immune cell.

In another embodiment, the gene editing technology introduces a mutation into
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   genetic locus or loci.

In an additional embodiment, the method of making the population of engineered immune cells as described herein using a gene editing technology to reduce the functional expression, relative to corresponding immune cells that have not been so engineered, of one or more targets as described herein by introducing a mutation into the genetic locus or loci or the one or more targets, comprises introducing into immune cells one or more genomic modifications that functionally impair or reduce expression of the one or more targets relative to immune cells without the one or more genomic modifications.

In another embodiment, the gene editing technology does not introduce a mutation into the β2m loci. In an embodiment, the mutation is any one or more of an insertion, e.g., the insertion of one or more nucleotides or base pairs, e.g., the insertion of a sequence that encodes a protein, a deletion of one or more nucleotides or base pairs, and a substitution of one or more nucleotides or base pairs.

In an embodiment of the method disclosed herein, the functional expression level of any one or more of TAP2, NLRC5, β2m, TRAC, CIITA, RFX5, RFXAP and RFXANK is measured by determining the surface expression level of an HLA protein, of beta2 microglobulin (B2M) or of both HLA and B2M on the surface of the engineered immune cell, or is measured by flow cytometry. In various embodiments of the present disclosure, the functional expression level of any one or more of CD48, CD58, and ICAM-1 is measured by determining the surface expression level of each cell surface protein, such as one or more of a CD48, a CD58 or an ICAM-1 protein on the surface of the engineered immune cell, or is measured by flow cytometry. In an embodiment, the extent of reduction in the surface expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   is determined relative to the corresponding expression level in a cell of the same type that has not been gene edited.

In an embodiment of the methods disclosed herein, the engineered immune cell expresses an additional protein, which can be any desired protein including an antigen binding protein or a CD70 binding protein (as further described herein). In an embodiment, the antigen binding protein comprises a CAR. In an embodiment, a nucleic acid encoding the CAR (and optionally a CD70 binding protein) is inserted into a disrupted CD58, CD48, ICAM-1, TAP2, NLRC5, β2m, TRAC, CIITA, RFX5, RFXAP or RFXANK locus or such an insertion disrupts the locus. In another embodiment, the antigen binding protein is a T cell receptor (TCR) component. In an embodiment, a nucleic acid encoding the TCR is inserted into a disrupted CD48, CD58, ICAM-1, TAP2, NLRC5, β2m, TRAC, CIITA, RFX5, RFXAP or RFXANK locus or such an insertion disrupts the locus.

In an embodiment, the method comprises reducing the functional expression level of the disrupted locus. In an embodiment, the method comprises reducing the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In an additional embodiment, the method of reducing the functional expression of the disrupted locus by reducing the functional expression level of one or more targets as described herein comprises introducing into immune cells one or more genomic modifications that functionally impair or reduce expression of the one or more targets relative to immune cells without the one or more genomic modifications.

In another embodiment, the method does not comprise reducing the functional expression level of β2m.

In certain embodiments, the method further comprises introducing into the engineered immune cell one or more genomic modifications of one or more of a gene encoding a TCR component e.g., a TCRa gene and a CD52 gene.

In another aspect, the present disclosure provides a method of reducing peptide diversity presented on the cell surface e.g., by MHC class I, the method comprising reducing the functional expression level of any one or more of TAP2, NLRC5, β2m, CIITA, RFX5, RFXAP and RFXANK, in some embodiments reducing this level to about 90% or less (in other words, a reduction of at least about 10%, e.g., to a level of about 90 or less compared to a control level of 100) or to about 75% or less of a comparable cell that has not been correspondingly altered. In addition to reducing peptide diversity, the method disclosed herein may also comprise the concurrent downregulation or elimination of certain cell surface receptors that are known to play a role in immune cell adhesion and activation at the immune synapse, e.g., one or more of CD48, CD58, and ICAM-1. In various embodiments, reducing the functional expression level of one or more genes, as described herein, comprises the use of a gene editing technology selected from the group consisting of TALEN, zinc fingers, Cas-CLOVER, and a CRISPR/Cas system (including for example, Cas9, Cas12 and MAD7). In another embodiment, the method comprises reducing the functional expression level of
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; or (vii) CD48, CD58, ICAM-1, RFX5, and NLRC5.

In an additional embodiment, the method of reducing peptide diversity presented on the cell surface and concurrently downregulating or eliminating certain cell surface receptors that are known to play a role in immune cell adhesion and activation at the immune synapse by reducing the functional expression level of one or more genes/targets as described herein comprises introducing into immune cells one or more genomic modifications that functionally impair or reduce expression of the one or more targets relative to immune cells without the one or more genomic modifications.

In another embodiment, the method does not comprise reducing the functional expression of β2m.

In an embodiment, the extent of reduction in the expression level of one or more of CD48, CD58, ICAM-1, TAP2, NLRC5, β2m, TRAC, CIITA, RFX5, RFXAP and RFXANK is determined relative to the corresponding expression level in a cell of the same type that has not been gene edited. In an embodiment, the functional expression level of one or more of TAP2, NLRC5, β2m, TRAC, CIITA, RFX5, RFXAP and RFXANK is measured by determining the surface expression level of HLA, beta2 microglobulin (B2M) or both HLA and B2M on the surface of the engineered immune cell. In various embodiments of the present disclosure, the extent of reduction in the expression level of any one or more of CD48, CD58, and ICAM-1 is measured by determining the surface expression level of each cell surface protein, such as one or more of a CD48, a CD58 or an ICAM-1 protein on the surface of the engineered immune cell, or is measured by flow cytometry.

In another aspect, the present disclosure provides a method of decreasing T cell-mediated killing of allogeneic cells comprising reducing the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.
   in an engineered immune cell such as an engineered T cell. In another embodiment, the method of decreasing T cell-mediated killing of allogeneic cells does not comprise reducing the functional expression level of β2m.

In an additional embodiment, the method of decreasing T cell-mediated killing of allogeneic cells by reducing the functional expression level of one or more targets as described herein in an engineered immune cell, such as an engineered T cell, comprises introducing into an immune cell, such as a T cell, one or more genomic modifications that functionally impair or reduce expression of the one or more targets relative to an immune cell without the one or more genomic modifications.

In some embodiments, the method results in a reduced level of expression, stated relative to the expression level in non-engineered immune cells, that is 0%, for example when both chromosomal copies of a gene are knocked out, or 50%, for example when one of the two chromosomal copies of a gene is knocked out and there is no compensatory increase in expression of the other chromosomal copy of that gene. In some embodiments, the method results in a reduced level of expression, stated relative to the expression level in non-engineered immune cells, that is between 0% and 90% or between any two values intermediate between 0% and 90%, for example between 10% and 90%, between 25% and 90%, between 25% and 75%, between 10% and 50%, between 25% and 50%, between 50% and 90%, and between 50% and 75%.

In some embodiments, reducing the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   comprises the use of a gene editing technology, for example, any one or more of TALEN, zinc fingers, Cas-CLOVER, and/or CRISPR/Cas system, and/or any one or more known knockdown methods e.g., those that employ any of various RNA-based techniques (e.g., shRNA, anti-sense RNA, miRNA, siRNA).

In another embodiment, the method comprises reducing the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In an additional embodiment, the method of reducing the functional expression level of one or more targets as described herein in immune cells by using a gene editing technology and/or any one or more known knockdown methods comprises introducing into immune cells one or more genomic modifications that functionally impair or reduce expression of the one or more targets relative to immune cells without the one or more genomic modifications.

In another embodiment, the method does not comprise reducing the functional expression level of β2m.

In an embodiment, the extent of reduction in the expression level of one or more of CD48, CD58, ICAM-1, TAP2, NLRC5, β2m, TRAC, CIITA, RFX5, RFXAP and RFXANK is determined relative to the corresponding expression level in a cell of the same type that has not been so altered and/or manipulated.

In an embodiment of the method of decreasing T cell-mediated killing of allogeneic cells disclosed herein, the functional expression level of one or more of TAP2, NLRC5, β2m, TRAC, CIITA, RFX5, RFXAP and RFXANK is measured by determining the surface expression level of HLA, beta2 microglobulin (B2M) or both HLA and B2M on the surface of the engineered immune cell. In an embodiment, the surface expression level of one or more of TAP2, NLRC5, β2m, TRAC, CIITA, RFX5, RFXAP and RFXANK is measured by flow cytometry. In various embodiments of the present disclosure, the functional expression level of any one or more of CD48, CD58, and ICAM-1 is measured by determining the surface expression level of each cell surface protein, such as one or more of a CD48, a CD58 or an ICAM-1 protein on the surface of the engineered immune cell, or is measured by flow cytometry. In an embodiment, the method comprises introducing one or more genomic modifications of one or more of a gene encoding a TCR component e.g., a TCRa gene and a CD52 gene. In another embodiment, the engineered immune cell further comprises a polynucleotide that encodes a CD70 binding protein and/or functionally expresses a CD70 binding protein, as described herein.

In some embodiments of the method of decreasing T cell-mediated killing of allogeneic cells disclosed herein, the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   in engineered immune cells of the present disclosure is assayed by measuring the degree to which the engineered immune cells survive in the presence of effector cells e.g., T cells, in comparison to the degree to which non-engineered, but otherwise comparable e.g., identical, immune cells survive under the same conditions. In another embodiment, the engineered immune cell(s) further comprises a polynucleotide that encodes a CD70 binding protein and/or functionally expresses a CD70 binding protein, as described herein.

In an aspect, the present disclosure provides an engineered immune cell that functionally expresses
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level.

In an additional embodiment, the engineered cell that functionally expresses one or more targets as described herein at a reduced level comprises one or more genomic modifications that functionally impair or reduce expression of the one or more targets relative to an immune cell without the one or more genomic modifications.

In another embodiment, the engineered immune cell does not functionally express β2m at a reduced level. In various embodiments, the cell exhibits a reduced level of expression of an MHC class I protein or complex at the cell surface, a reduced level of expression of an MHC class II protein or complex at the cell surface, or a reduced level of expression of an MHC class I protein or complex at the cell surface and a reduced level of expression of an MHC class II protein or complex at the cell surface. In various embodiments, the cell is a T cell. In another embodiment, the engineered immune cell further comprises a polynucleotide that encodes a CD70 binding protein and/or functionally expresses a CD70 binding protein, as described herein.

In various embodiments, the engineered immune cell disclosed herein further expresses an additional protein. In some embodiments, the additional protein is an antigen binding protein and/or a CD70 binding protein. In some embodiments, the antigen binding protein is a chimeric antigen receptor (CAR). In some embodiments, the antigen binding protein is a T cell receptor (TCR). In certain embodiments, the engineered immune cell comprises a nucleic acid encoding the additional protein. In some embodiments, the nucleic acid encoding the additional protein is located within a disrupted CD48, CD58, ICAM-1, TAP2, NLRC5, CIITA, RFX5, RFXANK, β2m or RFXAP locus and/or causes or creates a disruption in such a locus. In another embodiment, the nucleic acid encoding the additional protein is not located within a disrupted β2m locus and/or does not cause or create a disruption in such a locus.

In various embodiments, the engineered immune cell disclosed herein comprises or further comprises one or more genomic modifications of one or more of an endogenous TCRa (TCRa or TCR alpha) gene and an endogenous CD52 gene.

In various embodiments, the engineered immune cell disclosed herein is or is derived from an immune cell obtained from a healthy volunteer or a patient, or is derived from an iPSC.

In an aspect, the present disclosure provides a method of making an engineered immune cell disclosed herein comprising the use of a gene editing technology selected from the group consisting of TALENs, zinc fingers, Cas-CLOVER, and a CRISPR/Cas system to reduce functional expression of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In an additional embodiment, the method of making an engineered immune cell using a gene editing technology to reduce functional expression of one or more targets as described herein comprises introducing into immune cells one or more genomic modifications that functionally impair or reduce expression of the one or more targets relative to immune cells without the one or more genomic modifications.

In another embodiment, the method does not comprise use of a gene editing technology to reduce functional expression of β2m.

### BRIEF DESCRIPTION OF THE DRAWINGS

FIG. 1A depicts different components of the immune synapse (adapted from Huppa, J., Davis, M. T-cell-antigen recognition and the immunological synapse, Nat Rev Immunol 3, 973-983 (2003)).

FIG. 1B depicts immune evasion models of CD58-deficient tumor cells (see FIG. 8 of Challa-Malladi, M. et al., Combined genetic inactivation of 02-Microglobulin and CD58 reveals frequent escape from immune recognition in diffuse large B cell lymphoma. Cancer Cell. 2011 Dec. 13; 20(6):728-40, Epub 2011 Dec. 1)

FIG. 1C depicts how MHC-I expression controls the balance between T cell and NK cell rejection. Normal MHC-1 expression triggers T cell rejection (left panel) while the absence of MHC-1 triggers NK cell rejection via “missing self” elimination (right panel).

FIG. 1D depicts the use of a CD70 binding protein expressed on a CAR T cell against alloreactive host (e.g., patient) immune cells. The CD70 binding protein (also referred to as a dagger protein) binds to CD70 polypeptides expressed on host immune cells leading to their elimination. The CAR T cell expresses another molecule, e.g., CD58, at a reduced level compared to a non-engineered cell, thereby providing the CAR T cell with further protection against a host immune response.

FIG. 1E-1F shows the results of T cell MLR (E) and NK cell MLR (F) assays using cell populations having certain knockouts.

FIG. 2 shows the gene editing efficiency of cell populations having certain knockouts.

FIG. 3 shows the results of a T cell MLR assay using cell populations having certain knockouts.

FIG. 4 shows the results of a PMBC MLR assay using cell populations having certain knockouts.

FIG. 5A-5B shows the results of primed T MLR assays that were performed to test the effectiveness of CAR T cells against allogeneic T cell rejection. FIG. 5C-5D show the results of a further primed T MLR assay (C) and an NK cell MLR assay (D). FIG. 5E shows the results of a PBMC MLR assay. FIG. 5F shows that some CAR T cells with various knockouts were able to decrease host immune cell expansion (left panel: host CD8+ T cells; right panel: host CD4+ T cells). FIG. 5G shows that some CAR T cells with various knockouts were able to decrease host NK cell expansion. FIG. 5H shows the cytotoxicity of the CAR T cells with the different knockouts.

FIG. 6A-6B shows the results of an NK cell MLR assay that was performed to test the susceptibility of CAR T cells to allogeneic NK cell rejection. FIG. 6C demonstrates that the CAR T cells do not exhibit IL-2 independent growth.

FIG. 7 shows the gene editing efficiency for different CAR T cell populations.

FIG. 8 shows the expansion of different CAR T cell populations having different knockouts.

FIG. 9 shows the results of a cytotoxicity assay for the different CAR T cells having certain knockouts.

FIG. 10 shows the results of a primed T cell MLR assay for different engineered (e.g., gene-edited) CAR T cells.

FIG. 11 shows the results of a PBMC MLR assay for different engineered (e.g., gene-edited) CAR T cells.

FIG. 12A-12B shows the results of host immune cell expansion (A: host CD8+ T cells and CD4+ T cells; B: NK cells) in MLR assays with the different engineered (e.g., gene-edited) CAR T cells.

FIG. 13A-13D shows the results of different assays using CD19 CAR/CD70 binding protein/CD58 knockout cells. FIG. 13A shows the results of a PBMC MLR assay. FIG. 13B-13C shows the results of host cell expansion assay in the MLR assay (B: host CD4+ and CD8+ T cells; C: host NK cells). FIG. 13D shows the results of a cytotoxicity assay.

### DETAILED DESCRIPTION

The instant disclosure provides a gene editing strategy for providing a therapeutic allogeneic cell product that does not provoke, or provokes to a reduced degree, rejection by the recipient's immune system. This permits the cell product to persist longer in the recipient and thus promotes and/or improves the therapeutic effect. The present strategy involves downregulation or elimination of certain cell surface receptors that are known to play a role in immune cell adhesion and activation at the immune synapse, e.g., one or more of CD48, CD58, and ICAM-1. This strategy can be further supplemented with further downregulation or elimination of genes encoding molecules involved in HLA expression, which minimizes the diversity of peptides presented by an allogeneic cell product. This is achieved through the introduction of one or more genomic modifications (e.g., a genomic knockout or knockdown) of one or more of TAP2, NLRC5, β2m, CIITA, RFX5, RFXAP and RFXANK, the products of which function in peptide presentation by the cell. In one embodiment, the genomic modification (e.g., a genomic knockout or knockdown) is directed at
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; or (vi) CD48, CD58, ICAM-1, RFX5, and NLRC5.

In one embodiment, the one or more genomic modifications are at the genomic location of one or more genes (corresponding to one or more targets as described herein) or are elsewhere within the genome and not at the location of the one or more genes (corresponding to one or more targets as described herein), such that the modifications functionally impair or reduce expression of the one or more genes (corresponding to one or more targets as described herein).

An immune cell may be referred to as an engineered immune cell when it comprises at least one modification. Thus, the engineered immune cells of the present disclosure may be referred to as immune cells that comprise a genomic modification.

In one aspect, the engineered immune cells, as well as populations comprising the same demonstrate an improved ability to resist elimination by alloreactive immune cells. In one embodiment, the improved ability to resist elimination is demonstrated by an improved ability to survive in the presence of alloreactive immune cells such as PBMCs, T cells and/or natural killer (NK) cells. For example, the improved ability to survive can be shown via one or more mixed lymphocyte (MLR) assays as described herein, including in the Examples section.

In another aspect, the engineered immune cells, as well as the populations comprising the same demonstrate improved persistence in an allogeneic subject or an HLA-mismatched subject. In one embodiment, the improved persistence of the cells or populations is demonstrated by an improved ability to remain viable after being adoptively transferred into an allogeneic subject or an HLA-mismatched subject. As described herein, the cells and populations comprising the same are characterized by an improved ability to resist elimination by alloreactive immune cells. Without being bound by theory, following administration to an allogeneic subject or an HLA-mismatched subject, it is expected that the cells and populations having this improved ability will also demonstrate improved persistence.

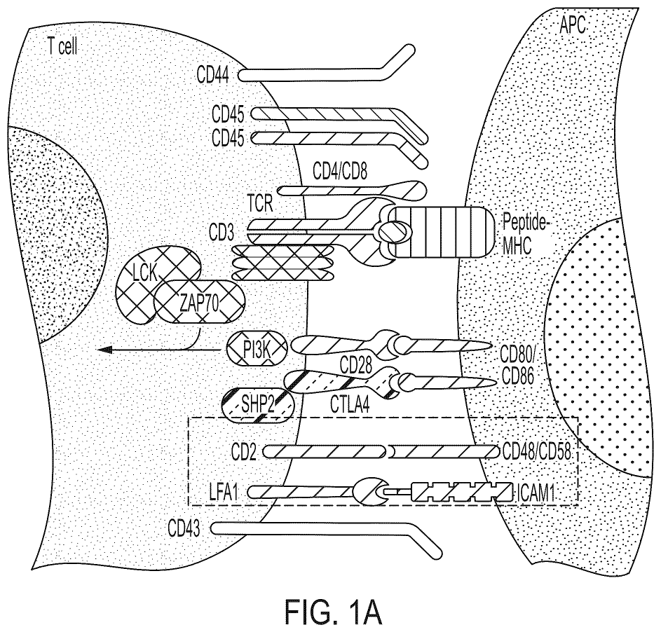

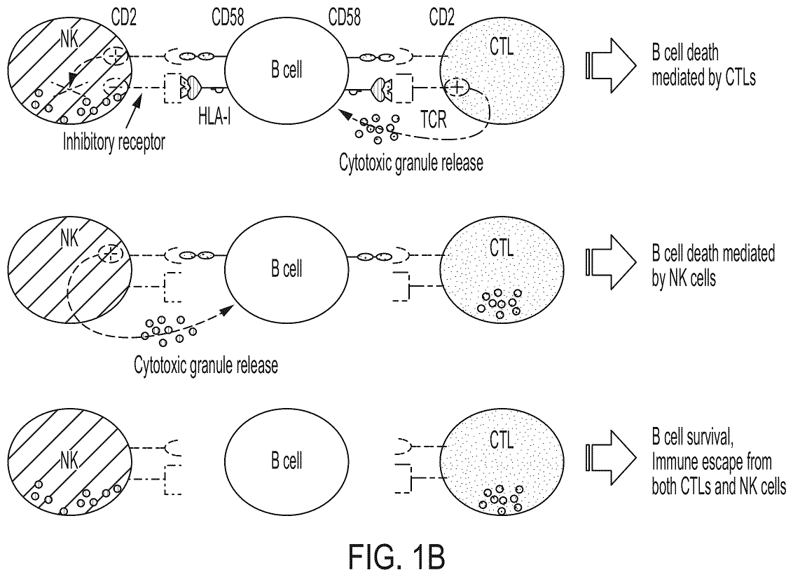

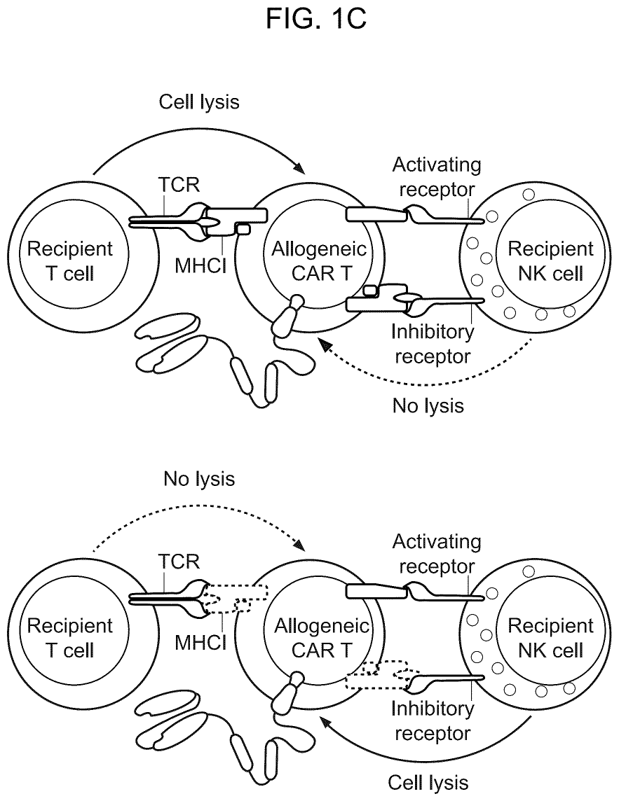

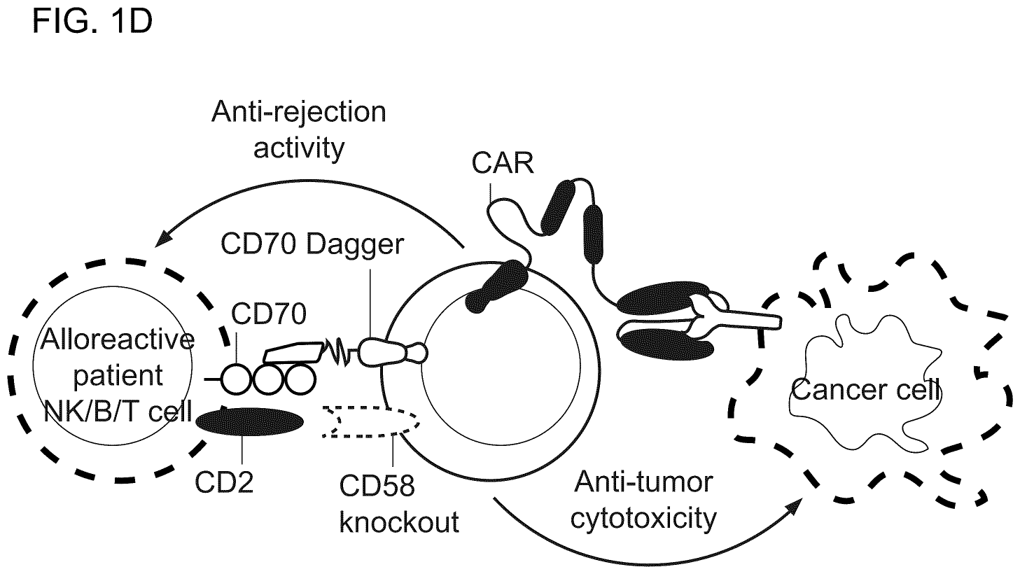

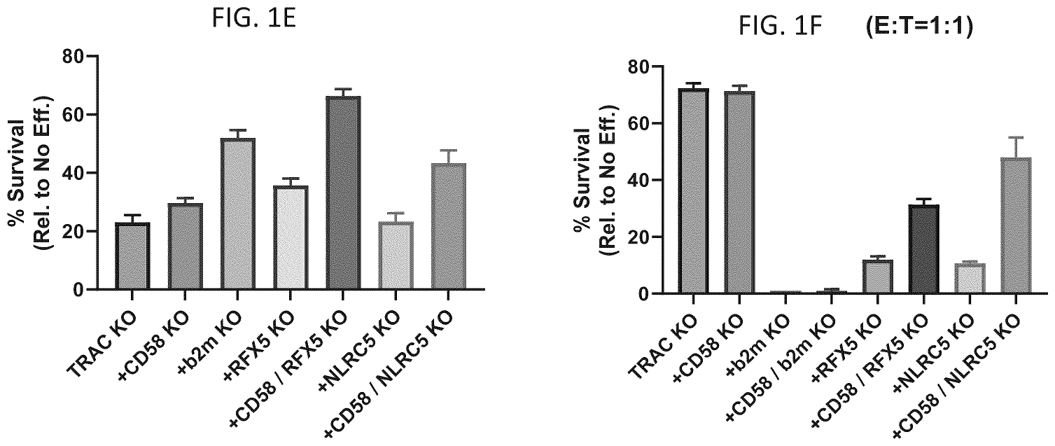

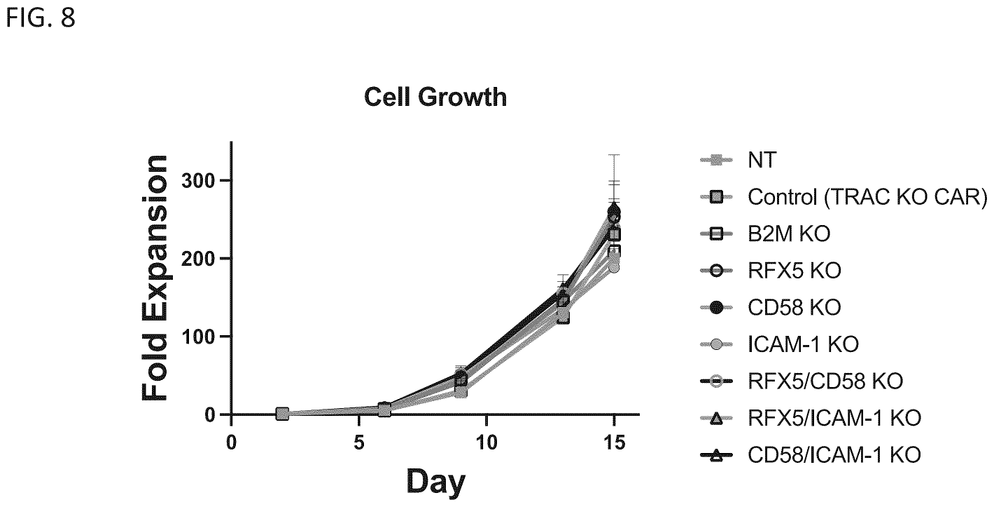

One aspect of the present invention concerns engineered cells (e.g., engineered immune cells, such as CAR T cells) that have functionally reduced expression (or no expression) of genes encoding cell surface molecules that could be recognized and subsequently killed by a recipient (host) immune cell, as well as methods for making and using such engineered cells. Certain cell surface molecules including, without limitation, CD58 and CD2, CD48 and CD2, and/or ICAM-1 and LFA-1, interact to provide proper cell adhesion and activation of immune cells (Dustin, M. L. The immunological synapse, Cancer Immunol Res. 2014 November; 2(11):1023-33). Furthermore, the loss of CD58 has been associated with resistance of tumor cells to T-cell mediated killing and immune evasion (Frangieh, C. J. et al. Multimodal pooled Perturb-CITE-seq screens in patient models define mechanisms of cancer immune evasion. Nat Genet. 2021 March; 53(3):332-341, Epub 2021 Mar. 1; Challa-Malladi, M. et al., Combined genetic inactivation of 02-Microglobulin and CD58 reveals frequent escape from immune recognition in diffuse large B cell lymphoma. Cancer Cell. 2011 Dec. 13; 20(6):728-40, Epub 2011 Dec. 1). For example, B cell lymphoma cells presenting tumor-associated antigens on HLA class I molecules can be recognized and subsequently killed by CD8 T cells (See FIG. 1B top panel—from FIG. 8 of Challa-Malladi, M. et al.). Tumor cells often downregulate HLA class I expression. However, HLA class I is a ligand for natural killer (NK) cell inhibitory responses such as KIRs (killer-cell immunoglobulin-like receptors), which transduce signals to counterbalance activation signals from receptors such as CD2 (see e.g., Jaeger, B. N., Vivier, E., Natural Killer Cell Tolerance: Control by Self or Self-Control? Cold Spring Harb Perspect Biol. 2012 March; 4(3):a007229-a007229). As a result, the loss of cell surface expression of HLA class I renders tumor cells vulnerable to NK cell killing via activation of CD2 by its ligand CD58 expressed on the tumor cells (See FIG. 1B middle panel—from FIG. 8 of Challa-Malladi, M. et al.). The concurrent loss of HLA class I and CD58 may confer protection from both T cell and NK cell killing thereby resulting in immune evasion by tumor cells (See FIG. 1B bottom panel—from FIG. 8 of Challa-Malladi, M. et al.). The functional reduction of expression (or no expression) of certain cell surface molecules, e.g., CD58, CD48, and ICAM-1, in engineered cells from a donor cell population (e.g., allogeneic engineered immune cells, such as CAR T cells) is a potential way to decrease the recognition and subsequent killing of the engineered cells by host T and NK cells following their infusion into a patient (i.e., an individual who is not the source of the donor cell population).

The present invention provides engineered cells (e.g., engineered immune cells, such as CAR T cells) having reduced functional expression (or no functional expression) of certain genes encoding cell surface molecules that play a role at the immune synapse, wherein the engineered cells can be part of an allogeneic cell graft administered to a patient in need (i.e., the host or recipient of the graft), which avoids or is capable of avoiding rejection by patient's immune cells. The modifications, e.g., gene editing modifications, to produce the engineered cells with reduced functional expression (or no expression) of certain genes does not require the exogenous expression of a protein, which is advantageous. For instance, gene editing approaches focused on introducing one or more exogenous genes into an engineered cell can be challenging due to the limited capacity of gene delivery vectors, e.g., vectors used to deliver CAR T and other CAR T cell enhancements, such as cytokines. Rather, the disclosure of the present invention includes methods for the modification of engineered cells during the manufacturing process using gene editing technologies, e.g., CRISPR-Cas9, TALEN, etc., as described herein, to inactivate or downregulate one or more genes encoding cell surface receptors (e.g., one or more of CD48, CD58, and ICAM-1). Such modifications can further include gene editing to downregulate or eliminate expression of genes involved in HLA molecules, as described herein. In another embodiment, the engineered cells (e.g., engineered immune cells, such as CAR T cells) further comprises a polynucleotide encoding a CD70 binding protein and/or functionally expresses a CD70 binding protein, as described herein.

In one other aspect of the present disclosure, the gene editing target is CD48 (also known as lymphocytic activation molecule 2 or SLAMF2), an immunoglobulin-like receptor that interacts with CD2 to contribute to the formation of an immunological synapse between T cells and antigen-presenting cells. In other aspects, the gene editing target is CD58 (also known as lymphocyte-function antigen 3 or LFA-3), a costimulatory receptor that interacts with its natural ligand of CD2, which also contributes to the formation of an immunological synapse. In an additional aspect, the gene editing target is Intercellular Adhesion Molecule 1 or ICAM-1 (also known as CD54), which (i) is a cell surface glycoprotein known for its role in stabilizing cell-cell interaction, (ii) is expressed in immune cells, and (iii) is a ligand for the LFA-1 receptor on leukocytes.

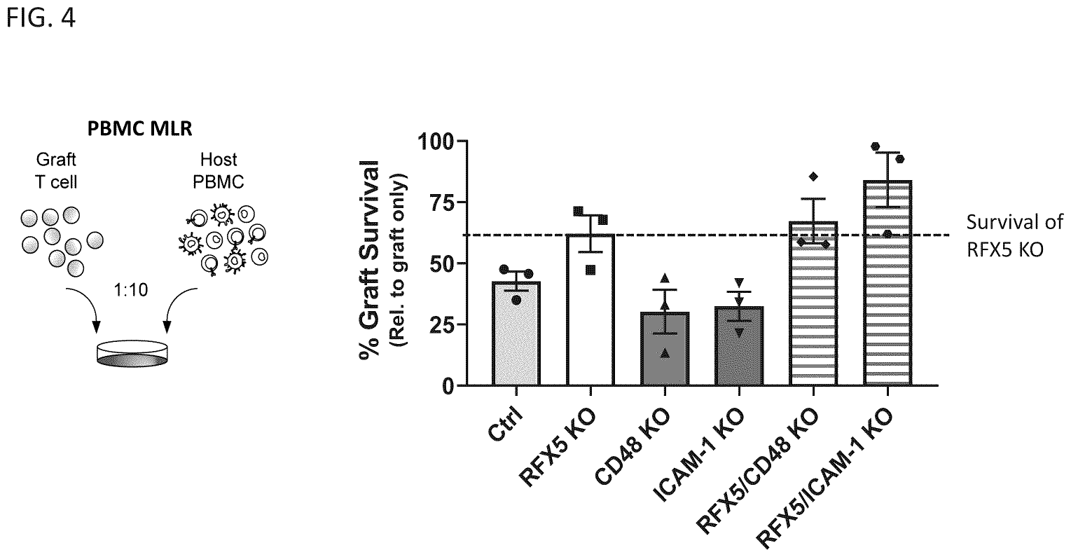

In one other aspect of the present disclosure, the gene editing target is the TAP2 component of the transporter associated with antigen processing (TAP). The dominant pathway by which MHC class I molecules are loaded with peptide is TAP-dependent: peptides generated by the proteasome (or the IFN-γ-inducible immunoproteasome) are imported to the endoplasmic reticulum (ER) via TAP and then loaded on MHC class I. A minority of peptides—substantially derived from signal peptides—are loaded through an alternative TAP- and proteasome-independent pathway following signal sequence cleavage by the ER-resident signal peptide peptidase (SPP). Knocking out TAP2 reduces surface β2m modestly (2-fold decrease after selection for KO cells) compared to the profound (10-100-fold) reduction in surface β2m in β2m KO cells (see FIG. 4A of PCT/US2022/14393, which is incorporated herein by reference in its entirety).

In another aspect of the present disclosure, the gene editing target may be a molecule involved in the regulation of transcription of HLA-I and HLA-II molecules. HLA-I and HLA-II molecules are tightly regulated at the transcriptional level by similar critical cis-regulatory elements: W/S, X1, X2 and Y box motifs. The Regulatory Factor, X (RFX) heterocomplex is composed of RFX5, RFXAP and RFXANK and binds to the X1 box. The X2 box is occupied by CREB/ATF1 family transcription factors and the Y box is bound by the NF-Y protein. In addition, two members of the nucleotide-binding domain and leucine-rich repeat containing receptor (NLR) family, NLRC5 and CIITA are required for formation of the HLA enhanceosome complex to promote transcription of HLA-I and HLA-II, respectively. NLRC5 and CIITA do not directly bind to DNA but rather require the aid of the other subunits of the enhanceosome for docking (Meissner, T. B. et al. NLR family member NLRC5 is a transcriptional regulator of MHC class I genes. Proc. Natl. Acad. Sci. U.S.A 107, 13794-13799 (2010); Meissner, T. B. et al. NLRC5 Cooperates with the RFX Transcription Factor Complex To Induce MHC Class I Gene Expression. J. Immunol. 188, 4951-4958 (2012). Specifically, NLRC5 associates with the RFXANK through its ankyrin repeats.

In one embodiment, the gene editing target is a member of the nucleotide-binding domain and leucine-rich repeat containing receptor (NLR) family called NLR caspase recruitment domain containing 5 (NLRC5). CRISPR/Cas9-mediated knockout of NLRC5 results in a 2.5-fold reduction in the level of surface β2m (see FIG. 4B of PCT/US2022/14393, which is incorporated herein by reference in its entirety). In another embodiment, the gene editing target is a RFX5, which along with RFXAP and RFXANK/B is part of the RFX complex that associates with the X1 box motif, as described herein.

The gene editing strategies disclosed herein surprisingly reduce peptide display sufficiently to reduce cell death at the hands of the recipient's T cell response while at the same time not reducing peptide display so much that killing by the recipient's NK cells is provoked. The strategies provided herein therefore represent a significant advance in allogeneic CAR-T therapy and other allogeneic cell therapies. The gene editing strategies provided herein confer the additional advantage that the NLRC5 knockout effect of suppressing MHC class I presentation should occur in the presence or absence of IFN-γ. This conclusion is supported by the finding that IFN-γ-induced MHC class I upregulation is dependent on NLRC5.

In one other aspect of the present invention, the gene editing target(s) of an engineered cell may be one or more genes encoding one or molecules that have a role in one or more cellular pathways the relate to rejection by host or recipient immune cells that are reactive with T cell and NK cell epitope determinants on the surface of an allogeneic cell product that is distinct from the host. In one embodiment, the gene editing target(s) of an engineered cell may be one or more genes encoding i) a cell surface receptor that is known to play a role in immune cell adhesion and activation at the immune synapse (e.g., one or more of CD48, CD58, and ICAM-1) and/or ii) a transcription factor or regulator of HLA-I and HLA-II molecules (e.g., RFX5, NLRC5, CIITA, RFXAP and RFXANK). The approaches described herein to generate engineered cells having reduced or eliminated expression of one or more genes may focus only on genes that encode molecules having a role at the immune synapse but may also be supplemented with reduced or eliminated expression of genes that encode molecules having a primary role as transcription factors for HLA-I and/or HLA-II molecules.

### GENERAL TECHNIQUES

The practice of the instant disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., TRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Gene editing techniques using TALENs, CRISPR/Cas9, and megaTAL nucleases, for example, are within the skill of the art and explained fully in the literature, such as T. Gaj et al., Genome-Editing Technologies: Principles and Applications, Cold Spring Harb Perspect Biol 2016; 8:a023754 and citations therein.

### Definitions

As used herein “autologous” means that cells, a cell line, or population of cells used for treating subjects that are obtained from said subject.

As used herein “allogeneic” means that cells or population of cells used for treating subjects that are not obtained from said subject, but instead from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, Regulatory T (Treg) cells, B cells, natural killer (NK) cells, natural killer T (NKT) cells, mast cells, and myeloid-derived phagocytes.

As used herein, the term “expression” refers to the transcription and/or translation of a particular nucleotide sequence driven by a promoter.

As used herein, “expression vector” refers to a vector comprising a recombinant polynucleotide comprising expression control sequences operatively linked to a nucleotide sequence to be expressed. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

Engineered immune cells of the present disclosure express e.g., functionally express one or more of CD48, CD58, ICAM-1, TAP2, NLRC5, β2m, TRAC, CIITA, RFX5, RFXAP and RFXANK at a reduced level as described herein and optionally further comprise additional features. For example, they can functionally express an antigen binding protein from an exogenous nucleic acid encoding the antigen binding protein introduced into the cell by techniques described herein. In one embodiment, the engineered immune cells of the present disclosure functionally express a first antigen binding protein, e.g., a CAR, and/or a second protein, e.g., CD70-binding protein. The engineered cells may also comprise genomic modifications e.g., mutations at endogenous genes such as TCRa and/or CD52 that decreases or eliminates functional expression of the gene, and/or they can express one or more additional proteins from an exogenous nucleic acid encoding the antigen binding protein introduced into the cell by techniques described herein. As described herein, engineered immune cells of the present disclosure can derive, e.g., be prepared from cells, e.g., immune cells obtained from various sources.

As used herein, to “functionally express” a gene means that a gene is expressed and that expression yields a functioning gene end product. For example, if a gene encodes a protein, then a cell functionally expresses the gene if expression of the gene ultimately produces a properly functioning protein. Thus, if a gene is not transcribed, or expression of the gene ultimately produces an RNA that is not translated or translation yields only a non-functioning protein e.g., the protein does not fold correctly or is not transported to its site of action (e.g., membrane, for membrane-bound proteins), for example, then the gene is not functionally expressed. Functional expression can be measured directly (e.g., by assaying for the gene product itself) or indirectly (e.g., by assaying for the effects of the gene product).

As used herein, “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

“Promoter” and “promoter sequence” are used interchangeably and refer to a DNA sequence capable of controlling the expression of a coding sequence or functional RNA. In general, a coding sequence is located 3′ to a promoter sequence. It is understood by those skilled in the art that different promoters can direct the expression of a gene in different tissues or cell types, or at different stages of development, or in response to different environmental or physiological conditions.

In any of the vectors of the present disclosure, the vector optionally comprises a promoter disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the instant disclosure.

The term “extracellular ligand-binding domain” as used herein refers to an oligo- or polypeptide that is capable of binding a ligand. Preferably, the domain will be capable of interacting with a cell surface molecule. For example, the extracellular ligand-binding domain can be chosen to recognize a ligand that acts as a cell surface marker on target cells associated with a particular disease state. The term “stalk domain” is used herein to refer to any oligo- or polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on a T cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1 BBL, OX40L, inducible costimulatory ligand (ICOS-L), intercellular adhesion molecule (ICAM, CD30L, CD40, CD70, CD83, HLA-G, MICA, M1 CB, HVEM, lymphotoxin β receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1 BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)2, and Fv), and any other modified configuration of the immunoglobulin molecule that comprises an antigen recognition site including, for example without limitation, single chain (scFv) and domain antibodies (including, for example, shark and camelid antibodies), and fusion proteins comprising an antibody. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen-binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (see, e.g., Ward et al., Nature 341:544-546, 1989), and an isolated complementarity determining region (CDR).

An antibody, an antibody conjugate, or a polypeptide that “specifically binds” to a target is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. It is also understood that by reading this definition, for example, an antibody (or moiety or epitope) that specifically binds to a first target may or may not specifically bind to a second target. As such, “specific binding” does not necessarily require (although it can include) exclusive binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are several techniques for determining CDRs, e.g., an approach based on cross-species sequence variability (i.e., Kabat et al. Sequences of Proteins of Immunological Interest, (5th ed., 1991, National Institutes of Health, Bethesda MD)); an approach based on crystallographic studies of antigen-antibody complexes (Al-lazikani et al., 1997, J. Molec. Biol. 273:927-948), the Chothia system (i.e., Chothia and Lesk, J. Mol. Biol. (1987) 196(4):901-917. As used herein, a CDR can refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs can be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs can also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs can be identified as the residues that make enthalpic contributions to antigen binding. See, e.g., Makabe et al., Journal of Biological Chemistry, 283:1 156-1 166, 2008. Still other CDR boundary definitions may not strictly follow one of the above approaches, but will nonetheless overlap with at least a portion of the Kabat CDRs, although they can be shortened or lengthened in light of prediction or experimental findings that particular residues or groups of residues or even entire CDRs do not significantly impact antigen binding. As used herein, a CDR can refer to CDRs defined by any approach known in the art, including combinations of approaches. The methods used herein can utilize CDRs defined according to any of these approaches. For any given embodiment containing more than one CDR, the CDRs can be defined in accordance with any of Kabat, Chothia, extended, AbM, contact, AHo and/or conformational definitions.

Antibodies of the instant disclosure can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide can comprise modified nucleotides, such as methylated nucleotides and their analogs. If present, modification to the nucleotide structure can be imparted before or after assembly of the chain. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the instant disclosure. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining a beneficial or desired clinical result. For purposes of the instant disclosure, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of subjects having a disease (e.g., cancer).

“Ameliorating” means a lessening or improvement of one or more symptoms as compared with not administering a treatment. “Ameliorating” also includes shortening or reduction in duration of a symptom. As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of the instant disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” can be considered in the context of administering one or more therapeutic agents, and a single agent can be considered to be given in an effective amount if, in conjunction with one or more other agents, a desirable result can be or is achieved.

As used herein, a “subject” is any mammal, e.g., a human, or a monkey. Mammals include, but are not limited to, farm animals, sport animals, pets, primates, horses, dogs, cats, mice and rats. In an exemplary embodiment, the subject is a human. In an exemplary embodiment, the subject is a monkey, e.g., a cynomolgus monkey.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions of the instant disclosure comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 21 st Ed. Mack Publishing, 2005).

As used herein, “alloreactivity” refers to the ability of T cells to recognize MIC complexes that were not encountered during thymic development. Alloreactivity manifests itself clinically as host-versus-graft rejection and graft-versus-host disease.

Reference to “about” a value or parameter herein includes (and describes) embodiments that are directed to plus or minus 1, 2, 3, 4, 5, 6, 7, 8, 9 or 10% that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the instant disclosure are described in terms of a Markush group or other grouping of alternatives, the instant disclosure encompasses not only the entire group listed as a whole, but also each member of the group individually and all possible subgroups of the main group, and also the main group absent one or more of the group members. The instant disclosure also envisages the explicit exclusion of one or more of any of the group members in the disclosed and/or claimed embodiments.

Unless otherwise defined, all technical and scientific terms used herein have the same meaning as commonly understood by one of ordinary skill in the art to which the present disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the instant disclosure. The materials, methods, and examples are illustrative only and not intended to be limiting.

An “antigen binding protein” comprises one or more antigen binding domains. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease or to a viral or bacterial antigen.

Antigen binding domains include, but are not limited to, antibody binding regions that are immunologically functional fragments. The term “immunologically functional fragment” (or “fragment”) of an antigen binding domain is a species of antigen binding domain comprising a portion (regardless of how that portion is obtained or synthesized) of an antibody that lacks at least some of the amino acids present in a full-length chain, but which is still capable of specifically binding to a target antigen. Such fragments are biologically active in that they bind to the target antigen and can compete with other antigen binding domains, including intact antibodies, for binding to a given epitope.

Immunologically functional immunoglobulin fragments include, but are not limited to, scFv fragments, Fab fragments (Fab′, F(ab′)2, and the like), one or more complementarity determining regions (“CDRs”), a diabody (heavy chain variable domain on the same polypeptide as a light chain variable domain, connected via a short peptide linker that is too short to permit pairing between the two domains on the same chain), domain antibodies, bivalent antigen binding domains (comprises two antigen binding sites), multispecific antigen binding domains, and single-chain antibodies. These fragments can be derived from any mammalian source, including but not limited to human, mouse, rat, camelid or rabbit. As will be appreciated by one of skill in the art, an antigen binding domain can include non-protein components.

The variable regions typically exhibit the same general structure of relatively conserved framework regions (FR) joined by the 3 hypervariable regions (CDRs). The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3.

In some embodiments, antigen binding domains comprise one or more complementarity binding regions (CDRs) present in the full-length light or heavy chain of an antibody, and in some embodiments comprise a single heavy chain and/or light chain or portion thereof. These fragments can be produced by recombinant DNA techniques or can be produced by enzymatic or chemical cleavage of antigen binding domains, including intact antibodies.

In some embodiments, the antigen binding domain is an antibody or fragment thereof, including one or more of the complementarity determining regions (CDRs) thereof. In some embodiments, the antigen binding domain is a single chain variable fragment (scFv), comprising light chain CDRs: CDR1, CDR2 and CDR3, and heavy chain CDRs: CDR1, CDR2 and CDR3.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196: 901-917; Al-Lazikani et al., (1997) J Mol Biol 273: 927-948; Chothia et al., (1992) J Mol Biol 227: 799-817; Tramontano et al., (1990) J Mol Biol 215(1): 175-82; and U.S. Pat. No. 7,709,226), contact numbering, the AbM scheme (Antibody Modeling program, Oxford Molecular) or the AHo system (Honneger and Pluckthun, J Mol Biol (2001) 309(3):657-70).

In some embodiments, the antigen binding domain is a recombinant antigen receptor. The term “recombinant antigen receptor” as used herein refers broadly to a non-naturally occurring surface receptor that comprises an extracellular antigen-binding domain or an extracellular ligand-binding domain, a transmembrane domain and an intracellular domain. In some embodiments, the recombinant antigen receptor is a chimeric antigen receptor (CAR). Chimeric antigen receptors (CARs) are well-known in the art. A CAR is a fusion protein that comprises an antigen recognition moiety, a transmembrane domain and T cell activation domains (see, e.g., Eshhar et al., Proc. Natl. Acad. Sci. USA, 90(2): 720-724 (1993)).

In some embodiments, the intracellular domain of a recombinant antigen receptor comprises a co-stimulatory domain and an ITAM-containing domain. In some embodiments, the intracellular domain of a recombinant antigen receptor comprises an intracellular protein or a functional variant thereof (e.g., truncation(s), insertion(s), deletion(s) or substitution(s)).

The term “extracellular ligand-binding domain” or “extracellular antigen-binding domain” as used herein refers to a polypeptide that is capable of binding a ligand or an antigen or capable of interacting with a cell surface molecule, such as a ligand or a surface antigen. For example, the extracellular ligand-binding or antigen-binding domain can be chosen to recognize a ligand that acts as a cell surface marker on target cells associated with a particular disease state, e.g., a tumor-specific antigen. In some embodiments, the antigen-binding domain comprises an antibody, or an antigen binding fragment or an antigen binding portion of an antibody. In some embodiments, the antigen binding domain comprises an Fv or scFv, an Fab or scFab, an F(ab′)2 or a scF(ab′)2, an Fd, a monobody, a affibody, a camelid antibody, a VHH antibody, a single domain antibody, or a darpin. In some embodiments, the ligand-binding domain comprises a partner of a binding pair, such as a ligand that binds to a surface receptor, or an ectodomain of a surface receptor that binds to a ligand.

The term “stalk domain” or “hinge domain” are used interchangeably herein to refer to any polypeptide that functions to link the transmembrane domain to the extracellular ligand-binding domain. In particular, stalk domains are often used to provide more flexibility and accessibility for the extracellular ligand-binding domain.

The term “intracellular signaling domain” refers to the portion of a protein which transduces the effector signal function signal and directs the cell to perform a specialized function.

### Vectors

Expression vectors and methods for the administration of polynucleotide compositions are known in the art and further described herein.

In another aspect, the instant disclosure provides a method of making any of the polynucleotides described herein.

Polynucleotides complementary to any such sequences are also encompassed by the instant disclosure. Polynucleotides can be single-stranded (coding or antisense) or double-stranded, and can be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences can, but need not, be present within a polynucleotide of the instant disclosure, and a polynucleotide can, but need not, be linked to other molecules and/or support materials.

Polynucleotides can comprise a native sequence (i.e., an endogenous sequence that encodes an antibody or a portion thereof) or can comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide can generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof. Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence can be compared to a reference sequence of the same number of contiguous positions after the two sequences are optimally aligned.

Optimal alignment of sequences for comparison can be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:1 1-17; Robinson, E. D., 1971, Comb. Theor. 1 1:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window can comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants can also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the instant disclosure. Further, alleles of the genes comprising the polynucleotide sequences provided herein are within the scope of the instant disclosure. Alleles are endogenous genes that are altered as a result of one or more mutations, such as deletions, additions and/or substitutions of nucleotides. The resulting mRNA and protein can, but need not, have an altered structure or function. Alleles can be identified using standard techniques (such as hybridization, amplification and/or database sequence comparison).

The polynucleotides of the instant disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further described herein. Polynucleotides can be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in, e.g., U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors can be constructed according to standard techniques, or can be selected from a large number of cloning vectors available in the art. While the cloning vector selected can vary according to the host cell intended to be used, useful cloning vectors will generally have the ability to self-replicate, can possess a single target for a particular restriction endonuclease, and/or can carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the instant disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components can generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding an antigen binding protein, e.g., a CAR, can exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, cause a ribosomal “skip” from one codon to the next without the formation of a peptide bond between the two amino acids encoded by the codons (see, e.g., Donnelly and Elliott 2001; Atkins, Wills et al. 2007; Doronina, Wu et al. 2008). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro-sequence or pre-sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences can be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. In some embodiments, nucleic acid sequences of the instant disclosure are codon-optimized for expression in mammalian cells, preferably for expression in human cells. Codon-optimization refers to the exchange in a sequence of interest of codons that are generally rare in highly expressed genes of a given species for codons that are generally frequent in highly expressed genes of such species, such codons encoding the same amino acids as the codons that are being exchanged.

### Methods of Preparing Engineered Cells

Methods of preparing engineered cells, e.g., engineered immune cells, for use in immunotherapy are provided herein. In some embodiments, the methods comprise introducing an antigen binding protein e.g., a CAR into one or more immune cells, or introducing a polynucleotide encoding the antigen binding protein e.g., CAR, and expanding the cells. In some embodiments, the instant disclosure relates to a method of engineering an immune cell comprising: providing an immune cell and expressing at the surface of the cell at least one antigen binding protein e.g., a CAR. In some embodiments, the method comprises: transfecting the cell with at least one polynucleotide encoding an antigen binding protein e.g., a CAR, and expressing the at least one polynucleotide in the cell.

In some embodiments, the polynucleotides encoding the antigen binding protein e.g., a CAR are present in one or more expression vectors for stable expression in the cells. In some embodiments, the polynucleotides are present in viral vectors for stable expression in the cells. In some embodiments, the viral vectors can be for example, lentiviral vectors or adenoviral vectors.

In some embodiments, polynucleotides encoding polypeptides according to the present disclosure can be mRNA which is introduced directly into the cells, for example by electroporation. In some embodiments, CytoPulse technology can be used to transiently permeabilize living cells for delivery of material into the cells. Parameters can be modified in order to determine conditions for high transfection efficiency with minimal mortality.

Also provided herein are methods of transfecting an immune cell e.g., a T cell. In general, any conventional method known to the person of ordinary skill in the art can be used, such as introducing any of RNA, DNA or protein into a cell by means of electroporation. See, e.g., Luft and Ketteler, J. Biomolec Screening 20(8): 932 (2015) (DOI: 10.1177/1087057115579638). In some embodiments, the method comprises: contacting a T cell with RNA and applying to the T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V per centimeter with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting a T cell comprises contacting said T cell with RNA and applying to the T cell an agile pulse sequence comprising: (a) an electrical pulse with a voltage of about 1600, 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250 to 3000 V per centimeter, e.g., of 2250, 2300, 2350, 2400, 2450, 2500, 2550, 2400, 2450, 2500, 2600, 2700, 2800, 2900 or 3000V per centimeter with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, the method can further comprise a step of genetically engineering a cell by inactivating or reducing the expression level of at least one gene expressing, for example without limitation, TAP2, NLRC5, β2m, CIITA, RFX5, RFXAP and RFXANK, a component of the TCR, a target for an immunosuppressive agent, an HLA gene, and/or an immune checkpoint protein such as, for example, PDCD1 or CTLA-4. By inactivating a gene, it is intended that the gene of interest is not expressed in a functional protein form. In some embodiments, the gene to be inactivated is one or more of the genes selected from the group consisting of, for example without limitation, NLRC5, TAP2, TCRa, TCRβ, β2-microglobulin (“β2m” or β2m), CD52, CIITA, RFX5, RFXAP, RFXANK, GR, deoxycytidine kinase (DCK), PD-1, and CTLA-4. In some embodiments the method comprises inactivating or reducing the expression level of one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease or TALEN®), a megaTAL nuclease or a Cas9 endonuclease.

In another aspect, a step of genetically modifying or engineering immune cells e.g., T cells can comprise: modifying immune cells e.g., T cells by inactivating at least one gene expressing a target for an immunosuppressive agent, and; expanding the cells, optionally in the presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others can act through activation of T cells or by inhibiting the activation of helper cells. The methods according to the instant disclosure allow conferring immunosuppressive resistance to e.g., T cells for immunotherapy by inactivating the target of the immunosuppressive agent in the T cells. As non-limiting examples, targets for an immunosuppressive agent can be a receptor for an immunosuppressive agent such as for example without limitation CD52, glucocorticoid receptor (GR), FKBP family gene members, and cyclophilin family gene members.

Compositions and methods for expressing an antigen binding protein e.g., a CAR, and/or a CD70 binding protein (as described herein) in conjunction with downregulation of functional expression of one or more of CD48, CD58, ICAM-1, TAP2, NLRC5, β2m, TRAC, RFX5, RFXAP, CIITA and RFXANK are provided herein. In other embodiments, the downregulation of functional expression is of (i) one or both of NLRC5 and RFX5 and (ii) one or more of CD58, CD48, and ICAM-1. In another embodiment, the downregulation of functional expression is of both NLRC5 and CD58, both RFX5 and CD58, both RFX5 and CD48, both NLRC5 and CD48, both RFX5 and ICAM-1, or both NLRC5 and ICAM-1. In one other embodiment, the downregulation of functional expression is of only one of CD48, CD58 and ICAM-1. In an additional embodiment, the downregulation of functional expression is of (i) CD58, NLRC5, and RFX5, (ii) CD48, NLRC5, and RFX5, (iii) ICAM-1, NLRC5, and RFX5, (iv) CD58, ICAM-1, and RFX5, (v) CD48, ICAM-1, and RFX5, (vi) CD58, CD48, and RFX5, (vii) CD58, ICAM-1, and NLRC5, (viii) CD48, ICAM-1, and NLRC5, or (ix) CD58, CD48, and NLRC5. Also provided are uses of such compositions and methods for improving the functional activities of immune cells e.g., T cells, such as CAR-T cells. The methods and compositions provided herein are useful for improving in vivo persistence and therapeutic efficacy of engineered immune cells e.g., engineered T cells such as CAR-T cells.

### Engineered Cells

Engineered cells, such as engineered immune cells e.g., engineered T cells provided herein may express an antigen binding protein e.g., a chimeric antigen receptor (CAR), and/or a CD70 binding protein, and express any one or more of CD48, CD58, ICAM-1, TAP2, NLRC5, β2m, TRAC, CIITA, RFX5, RFXAP and RFXANK at a level not greater than 75%, not greater than 50%, not greater than 25%, or not greater than 10% of the expression level in non-engineered immune cells. Advantageously, the engineered immune cells provided herein exhibit improved in vivo persistence and/or increased resistance to rejection by the recipient's immune system, relative to non-engineered cells.

In some embodiments, an engineered cell, e.g., an engineered immune cell, comprises a population of CARs, each CAR comprising different extracellular antigen-binding domains. In some embodiments, an engineered cell, e.g., an engineered immune cell, comprises a population of CARs, each CAR comprising the same extracellular binding domains.

In one embodiment, the engineered cell, e.g., engineered immune cell, is a T cell (e.g., inflammatory T lymphocyte, cytotoxic T lymphocyte, regulatory T lymphocyte (Treg), helper T lymphocyte, tumor infiltrating lymphocyte (TIL)), natural killer T cell (NKT), TCR-expressing cell, dendritic cell, killer dendritic cell, a mast cell, or a B-cell. In some embodiments, the engineered cell, e.g., engineered immune cell, can be derived from CD4+T-lymphocytes or CD8+T-lymphocytes. In another embodiment, the engineered cell may be derived from a population of T-lymphocytes that contains CD4+T-lymphocytes and CD8+T-lymphocytes. In some exemplary embodiments, the engineered immune cell is a T cell. In some exemplary embodiments, the engineered immune cell is a gamma delta T cell. In some exemplary embodiments, the engineered immune cell is a macrophage. In some exemplary embodiments, the engineered immune cell is a natural killer (NK) cell.

In some embodiments, the engineered cell, e.g., the engineered immune cell, can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, the engineered cell, e.g., the engineered immune cell, is obtained or prepared from peripheral blood. In some embodiments, the engineered cell is obtained or prepared from peripheral blood mononuclear cells (PBMCs). In some embodiments, the engineered cell is obtained or prepared from bone marrow. In some embodiments, the engineered cell is obtained or prepared from umbilical cord blood. In some embodiments, the cell is a human cell. Representative human cells are CD34+ cells.

In some embodiments, the cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

Any immune cell capable of expressing heterologous DNAs can be used for the purpose of expressing the antigen binding protein e.g., CAR of interest and further for engineering to express a reduced level of one or more of CD48, CD58, ICAM-1, TAP2, NLRC5, β2m, TRAC, CIITA, RFX5, RFXAP and RFXANK.

In some embodiments, an immune cell e.g., a T cell provided herein further is modified e.g., genetically modified to express one or more of CD48, CD58, ICAM-1, TAP2, NLRC5, β2m, TRAC, CIITA, RFX5, RFXAP and RFXANK at a reduced level relative to a comparable cell that has not been so modified. For example, the immune cells can be genetically modified to knock out all or part of one or more of the CD48, CD58, ICAM-1, TAP2, NLRC5, β2m, TRAC, CIITA, RFX5, RFXAP and RFXANK loci such that the corresponding functional protein is not expressed at the cell's surface, e.g., by deleting genomic DNA that comprises part or all of the entire coding sequence of the locus and/or the genomic DNA that comprises the locus's transcriptional control and/or promoter and/or activation elements and/or by introducing an insertion, deletion or substitution mutation that prevents production of a functional protein. In addition, the immune cells can be genetically modified to knock out all or part of the loci of
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, or both NLRC5 and ICAM-1;
only one of CD48, CD58 and ICAM-1; or
(i) CD58, NLRC5, and RFX5, (ii) CD48, NLRC5, and RFX5, (iii) ICAM-1, NLRC5, and RFX5, (iv) CD58, ICAM-1, and RFX5, (v) CD48, ICAM-1, and RFX5, (vi) CD58, CD48, and RFX5, (vii) CD58, ICAM-1, and NLRC5, (viii) CD48, ICAM-1, and NLRC5, or (ix) CD58, CD48, and NLRC5,
   such that the corresponding functional protein is not expressed at the cell's surface, e.g., by deleting genomic DNA that comprises part or all of the entire coding sequence of the locus and/or the genomic DNA that comprises the locus's transcriptional control and/or promoter and/or activation elements and/or by introducing an insertion, deletion or substitution mutation that prevents production of a functional protein.

In one embodiment, the one or more genomic modifications are at the genomic location of one or more genes (corresponding to one or more targets as described herein) or are elsewhere within the genome and not at the location of the one or more genes (corresponding to one or more targets as described herein), such that the modifications functionally impair or reduce expression of the one or more genes (corresponding to one or more targets as described herein).

In another embodiment, the engineered immune cell further comprises a polynucleotide encoding a CD70 binding protein and/or functionally expresses a CD70 binding protein. In some embodiments, the CD70-binding proteins provided herein comprise an extracellular domain (e.g., a single chain variable fragment (scFv)) and a transmembrane domain. In some embodiments, the CD70-binding protein provided herein comprise an extracellular ligand-binding domain (e.g., scFv), a transmembrane domain, and an intracellular signaling domain. In some embodiments, the CD70-binding protein comprises one or more intracellular signaling domains selected from the group consisting of a CD3C signaling domain, a CD36 signaling domain, a CD3γ signaling domain, a CD38 signaling domain, a CD28 signaling domain, a CD2 signaling domain, an OX40 signaling domain, and a 4-1BB signaling domain, or a variant thereof. In one other embodiment, the CD70-binding protein comprises an intracellular signaling domain that comprises a CD3C signaling domain. In some embodiments, the CD70-binding protein is a CAR.

In some embodiments, the intracellular signaling domain comprises the amino acid sequence of one or more of SEQ ID NOs: 1, 7-14, 17-70, or 89-90. In some embodiments, the intracellular signaling domain comprises the amino acid sequence of one or more of SEQ ID NO: 7, 89, 8, 90, 12, 11, 61, 62, 64, or 65. In some embodiments, the CD70 binding protein comprises a CD3C or a CD3γ signaling domain, or a variant thereof, and does not comprise a costimulatory domain. In some embodiments, the CD70 binding protein comprises a 4-1BB signaling domain, or a variant thereof, and does not comprise a CD3 signaling domain. In some embodiments, the CD70 binding protein comprises a 4-1BB signaling domain and a CD3C signaling domain. In some embodiments, the CD70 binding protein does not comprise an intracellular signaling domain. Different intracellular signaling domain or combination thereof can confer different signaling strength that can contribute to T cell proliferation, potency, survival, persistence, and/or resistant to host immune cell rejection. Described herein are CD70-binding proteins comprising no to one or more intracellular signaling domains. In another embodiment, the CD70 binding protein comprises an scFv having an amino acid sequence shown as SEQ ID NO: 82 or 85. In one other embodiment, the CD70 binding protein comprises an amino acid sequence shown as SEQ ID NO: 86. Any of SEQ ID NOs: 1, 7-14, 17-70, or 89-90 may comprise one or more substitutions, insertions, or deletions. These sequences may contain 5, 4, 3, 2 or fewer substitutions, insertions, or deletions. The polypeptides including such substitutions, insertions, or deletions may retain their activity. For instance, the intracellular signaling domains retain the ability to transduce the relevant signal and the CD70 binding protein retains the ability to specifically bind to CD70.

In some embodiments, engineered immune cells that have been engineered to functionally express a reduced level (relative to corresponding cells that have not been so engineered) of one or more targets described herein and that have been further engineered to express a CD70-binding protein can exhibit different levels of persistence and/or resistance to rejection by host immune cells and can be suitable for use in lymphodepletion in vivo when administered to a patient. In some embodiments, engineered immune cells that functionally express the CD70-binding proteins described herein can inhibit proliferation and/or activities of host immune cells to different degrees that can allow for fine-tuning of the depth of lymphodepletion in vivo when administered to a patient. For example, engineered immune cells that functionally express a CD70-binding protein that demonstrated extended expansion and/or inhibition of host immune cells proliferation or activities, in e.g., an MLR assay, can be used for an extended lymphodepletion. In contrast, engineered immune cells that functionally express a CD70-binding protein that demonstrated less extended expansion and/or inhibition of host immune cells in the same or similar assay can be used when a less complete or a less thorough lymphodepletion is desired.

In one aspect, the reduced level of expression relative to a comparable cell that has not been modified may be a knockout or a knockdown of expression. Different knockdown methods may be suitable, such as those that employ any of various RNA-based techniques (e.g., short hairpin RNA (shRNA), antisense RNA, microRNA (miRNA), small (or short) interfering RNA (siRNA); see, e.g., Van Hoeck et al., Biomaterials, Vol. 286, July 2022, 121510, ISSN 0142-9612; Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016), as well as Krause et al. U.S. Pat. No. 9,556,433, which is incorporated herein by reference in its entirety). An RNA-based reagent, e.g., an RNA interference molecule, may be delivered to a cell, e.g., an immune cell, such that one or more genes are knocked down. In one embodiment, the RNA-based reagent may be configured to target or is targeted to the one or more genes. In one other embodiment, the RNA interference molecule comprises an RNA interference sequence, which comprises one or more sequences that are complementary to one or more target genes. Alternatively, a polynucleotide comprising one or more RNA interference sequences directed to one or more target genes may be inserted into the genome. In one embodiment, the insertion may occur at a location in the genome that is not the location of the one or more target genes. In one embodiment, the RNA-based reagent is configured to target or is targeted to
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.
   In another embodiment, the RNA-based reagent is not configured to target β2m or is not targeted to β2m.

In some embodiments, functional expression levels of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   in immune cells e.g., T cells of the instant disclosure can be decreased by at least about 30%, 35%, 40%, 45%, 50%, 55%, 60%, 65%, 70%, 75%, 80%, 85%, 90%, 95%, 99%, or 100% relative to functional expression levels in comparable cells that have not been engineered to reduce the corresponding expression level. In another embodiment, the functional expression level of β2m is not decreased.

One or more antigen binding proteins e.g., one or more CARs, and/or CD70 binding proteins can be synthesized in situ in the cell after introduction of a polynucleotide construct encoding the proteins into the cell. Alternatively, an antigen binding protein e.g., CAR, and/or CD70 binding protein can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses, including lentiviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments, an engineered immune cell e.g., a T cell of the instant disclosure can comprise at least one antigen binding protein e.g., CAR, and/or a CD70 binding protein. The engineered immune cell e.g., T cell is further modified e.g., genetically engineered to express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.
   In one embodiment, the engineered immune cell e.g., a T cell, is not further modified to express a reduced level of β2m. In another embodiment, the engineered immune cell, such as a CAR T cell, further comprises a polynucleotide encoding a CD70 binding protein and/or functionally expresses a CD70 binding protein, as described herein.

In one other embodiment, the disclosure provides an engineered immune cell, e.g., a CAR T cell, or a population of engineered immune cells comprising the engineered immune cell that comprises one or more genomic modifications that functionally impair or reduce expression of one or more targets described herein. In one embodiment, the one or more genomic modifications are at the genomic location of one or more genes (corresponding to one or more targets as described herein) or are elsewhere within the genome and not at the location of the one or more genes (corresponding to one or more targets as described herein), such that the modifications functionally impair or reduce expression of the one or more genes (corresponding to one or more targets as described herein).

In some embodiments, the engineered immune cell e.g., an engineered T cell can comprise two or more different antigen binding proteins, e.g., two or more different CARs, each CAR comprising different extracellular ligand-binding domains, and/or a CD70 binding protein.

In some embodiments of an engineered immune cell e.g., T cell provided herein, a CAR that the cell expresses can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR. In another embodiment, the engineered immune cell further comprises or functionally expresses a CD70 binding protein, as described herein.

The extracellular ligand-binding domain specifically binds to a target of interest. The extracellular ligand-binding domain may specifically bind a tumor antigen. For instance, the extracellular ligand-binding domain may specifically bind a tumor antigen selected from an oncofetal, overexpressed, tissue restricted, cancer-testis, or onco-viral antigen. The tumor antigen may be associated with a liquid tumor. In some embodiments, the target of interest can be any molecule of interest, including, for example, without limitation, BCMA, EGFRvIII, Flt-3, WT-1, CD20, CD23, CD30, CD38, CD70, CD33, CD133, WT1, TSPAN10, MHC-PRAME, Liv1, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17, Muc3, Muc3, Muc16, FAP alpha (Fibroblast Activation Protein alpha), Ly6G6D (Lymphocyte antigen 6 complex locus protein G6d, c6orf23, G6D, MEGT1, NG25), RNF43 (E3 ubiquitin-protein ligase RNF43, RING finger protein 43), specifically including the human form of any of the listed exemplary targets.

In some embodiments, the antigen binding domain specifically binds BCMA, MUC16 (also known as CA125), EGFR, EGFRvIII, MUC1, Flt-3, WT-1, CD20, CD23, CD30, CD38, CD70, CD33, CD133, MHC-WT1, TSPAN10, MHC-PRAME, MIC-NY-ESO1, HER2 (ERBB2), CAIX (Carbonic anhydrase IX), LIV1, ADAM10, CHRNA2, LeY, NKG2D, CS1, CD44v6, ROR1, CD19, Claudin-18.2 (Claudin-18A2, or Claudin18 isoform 2), PSCA, DLL3 (Delta-like protein 3, Drosophila Delta homolog 3, Delta3), Mud 7 (Mucin17, Muc3, Muc3), FAP alpha (Fibroblast Activation Protein alpha), Ly6G6D (Lymphocyte antigen 6 complex locus protein G6d, c6orf23, G6D, MEGT1, NG25), PSMA, MSLN, or RNF43 (E3 ubiquitin-protein ligase RNF43, RING finger protein 43). CARs and/or antibodies that target the antigens are disclosed, for example, in the following: BCMA—WO201616630, WO2020150339, WO2019196713, WO2016014565, WO2017025038; MUC16: U.S. Pat. No. 9,169,328, WO2016149368, WO2020023888; EGFRvIII: WO2017125830, WO2016016341; Flt3: WO2018222935, WO2020010284, WO2017173410; CD20: WO2018145649, WO2020010235, WO2020123691; CD38: WO2017025323; CD70: WO2019152742, WO2018152181; CD33: WO2016014576; CD133: WO2018072025; CS1: WO2019030240; ROR1: WO2016115559; CD19: WO2002077029, U.S. Pat. No. 11,077,144; Claudin: WO2018006882, WO2021008463; DLL3: WO2020180591; WT1: US20160152725A1, U.S. Pat. No. 7,622,119B2; CD23: U.S. Pat. No. 6,011,138A, CN1568198A; CD30: U.S. Ser. No. 10/815,301B2, U.S. Ser. No. 10/808,035B2; PRAME: US20180148503A1, WO2020186204A1; LIV1: US20200231699A1; NKG2D: WO2021179353A1, US20210269501A1; FAP Alpha: US20200246383A1, US20210115102A1; PSMA: US20210277141A1, WO2020108646A1; MSLN: CN109680002A, CN109628492A.

In some embodiments, the extracellular ligand-binding domain comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 72), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid or other vector containing a polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the instant disclosure is responsible for intracellular signaling following the binding of extracellular ligand-binding domain to the target resulting in the activation of the immune cell and immune response. The intracellular signaling domain has the ability to activate at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Examples of ITAM used in the instant disclosure can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain. In some embodiments the intracellular signaling domain of the CAR of the instant disclosure comprises a domain of a co-stimulatory molecule.

In some embodiments, the intracellular signaling domain of a CAR of the instant disclosure comprises a part of a co-stimulatory molecule selected from the group consisting of fragment of 4-1BB (GenBank: AAA53133) and CD28 (NP_006130 and isoforms thereof).

CARs are expressed on the surface membrane of the cell. Thus, the CAR can comprise a transmembrane domain. Suitable transmembrane domains for a CAR disclosed herein have the ability to (a) be expressed at the surface of a cell, for example an immune cell such as, for example without limitation, lymphocyte cells (e.g., T cells) or Natural killer (NK) cells, and (b) interact with the ligand-binding domain and intracellular signaling domain for directing a cellular response of an immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a domain of the T cell receptor such as α, β, γ or δ, polypeptide constituting CD3 complex, IL-2 receptor e.g., p55 (α chain), p75 (β chain or γ chain), subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain can comprise up to 300 amino acids, for example, from 10 to 100 amino acids or 25 to 50 amino acids. The stalk region can be derived from all or part of naturally occurring molecules, such as from all or part of the extracellular region of CD8, CD4, or CD28, or from all or part of an antibody constant region. Alternatively, the stalk domain can be a synthetic sequence that corresponds to a naturally occurring stalk sequence or can be an entirely synthetic stalk sequence. In some embodiments said stalk domain is a part of human CD8α chain (e.g., NP_001139345 and isoforms thereof). In another particular embodiment, the transmembrane domain comprises a part of the human CD8α chain. In some embodiments, CARs disclosed herein can comprise an extracellular ligand-binding domain that specifically binds BCMA, CD8α human stalk and transmembrane domains, the CD3ζ signaling domain, and 4-1BB signaling domain. In some embodiments, a CAR can be introduced into an immune cell as a transgene via a vector e.g., a plasmid vector. In some embodiments, the vector e.g., plasmid vector can also contain, for example, a selection marker which provides for identification and/or selection of cells which received the vector.

The CAR and/or CD70 binding protein polypeptides can be synthesized in situ in the cell after introduction of polynucleotides encoding the CAR and/or CD70 binding protein polypeptides into the cell. Alternatively, CAR and/or CD70 binding protein polypeptides can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses (e.g., lentiviruses), adenoviruses), liposomes, and the like. Transient transformation methods include, for example without limitation, microinjection, electroporation or particle bombardment. Polynucleotides can be included in vectors, such as for example plasmid vectors or viral vectors.

Also provided herein are immune cells e.g., T cells such as isolated T cells obtained according to any one of the methods described herein. Any immune cell capable of expressing heterologous DNAs can be used for the purpose of expressing the antigen binding protein e.g., CAR of interest, and/or the CD70 binding protein, and further for engineering to express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.

In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK− cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+T-lymphocytes and CD8+T-lymphocytes. In another embodiment, the engineered cell may be derived from a population of T-lymphocytes that contains CD4+T-lymphocytes and CD8+T-lymphocytes.

In some embodiments, the immune cells e.g., T cells such as isolated T cells are further modified e.g., genetically engineered by methods described herein (e.g., known gene editing techniques that employ, for example, TALENs, CRISPR/Cas9, or megaTAL nucleases to partially or wholly delete or disrupt
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5.
   gene loci) so that they express a reduced level of the corresponding protein relative to comparable cells not engineered to express a reduced or altered level of the corresponding protein. In another embodiment, the immune cells are not further modified e.g., genetically engineered by methods described herein (e.g., known gene editing techniques that employ, for example, TALENs, CRISPR/Cas9, or megaTAL nucleases to partially or wholly delete or disrupt β2m loci). In another embodiment, the immune cells (e.g., engineered immune cells, such as CAR T cells) further comprise a polynucleotide encoding a CD70 binding protein and/or functionally expresses a CD70 binding protein, as described herein.

In one other embodiment, the disclosure provides modified or engineered immune cells that comprises one or more genomic modifications that functionally impair or reduce expression of one or more targets described herein. In one embodiment, the one or more genomic modifications are at the genomic location of one or more genes (corresponding to one or more targets as described herein) or are elsewhere within the genome and not at the location of the one or more genes (corresponding to one or more targets as described herein), such that the modifications functionally impair or reduce expression of the one or more genes (corresponding to one or more targets as described herein).

The engineered immune cells provided herein can comprise one or more mimotope sequences that enable sorting of cells to enrich a population for cells engineered as described herein, e.g., cells that express the antigen binding protein, and/or that provide a safety switch mechanism to inactivate the immune cell after the cells have been administered to the patient or recipient, e.g., to limit adverse effects. Such mimotope sequences and their application in cell sorting and as safety switches are known in the art and described, for example, in US2018/0002435, which is incorporated herein by reference in its entirety.

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, can be used. In some embodiments, cells can be derived from a healthy donor, from a subject diagnosed with cancer or from a subject diagnosed with an infection. In some embodiments, cells can be part of a mixed population of cells which present different phenotypic characteristics.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8+ cells are further sorted into naive, stem cell memory, central memory, and effector cells by identifying characteristic cell surface antigens that are associated with each of these types of CD8+ cells. In some embodiments, the expression of phenotypic markers of central memory T cells include CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin.

In certain embodiments, CD4+ T cells are further sorted into subpopulations. For example, CD4+T helper cells can be sorted into naive, central memory, and effector cells by identifying cell populations that have characteristic cell surface antigens.

Also provided herein are cell lines obtained from a modified e.g., transformed or engineered immune cell e.g., engineered T cell according to any of the methods described herein. In some embodiments, an engineered immune cell e.g., engineered T cell according to the instant disclosure comprises a polynucleotide encoding an antigen binding protein e.g., a CAR, and/or a CD70 binding protein, and further modified or engineered e.g., genetically modified to express e.g., functionally express
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level. In another embodiment, the engineered immune cell is not further modified or engineered e.g., genetically modified to express e.g., functionally express β2m. In another embodiment, the engineered immune cell further comprises a polynucleotide encoding a CD70 binding protein and/or functionally expresses a CD70 binding protein, as described herein.

In a particular embodiment, there is provided a T cell comprising a genomic modification that functionally impairs or reduces expression of RFX5 and CD58 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, or CD8+T-lymphocytes.

In another particular embodiment, there is provided a T cell comprising a genomic modification that functionally impairs or reduces expression of NLRC5 and CD58 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of RFX5 and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of NLRC5 and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of CD58 and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of RFX5, NLRC5 and CD58 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of RFX5, NLRC5 and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of RFX5, CD58, and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

In one other particular embodiment, there is provided a T cell comprising a genomic modification that functionally impairs or reduces expression of NLRC5, CD58, and ICAM-1 relative to a T cell without the genomic modification, wherein the T cell also comprises: a CAR capable of specifically binding to a tumor antigen, optionally a CD70-specific CAR, optionally reduced or ablated expression of CD52, and reduced or ablated expression of endogenous TCR. The T cell may be an allogeneic T cell in relation to a subject to be treated. The T cell may be derived from a healthy donor. The T cell may be derived from the group consisting of PBMCs, CD4+T-lymphocytes, and CD8+T-lymphocytes.

### Methods Involving Engineered Immune Cells

The immune cells, e.g., T cells of the instant disclosure, can be activated and expanded, either prior to or after modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Immune cells e.g., T cells can be expanded in vitro or in vivo. Generally, the immune cells of the instant disclosure can be expanded, for example, by contact with an agent that stimulates a CD3 TCR complex and a co-stimulatory molecule on the surface of the immune cells to create an activation signal for the cell. For example, chemicals such as calcium ionophore A23187, phorbol 12-myristate 13-acetate (PMA), or mitogenic lectins like phytohemagglutinin (PHA) can be used to create an activation signal for the immune cell, e.g., a T cell.

In some embodiments, T cell populations can be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate medium (e.g., Minimal Essential Media, RPMI Media 1640 or, X-VIVO™ 5, (Lonza)) that can contain factors necessary for proliferation and viability, including serum (e.g., fetal bovine or human serum), interleukin-2 (IL-2), insulin, IFN-7, IL-4, IL-7, GM-CSF, IL-10, IL-2, IL-15, a TGFβ, and TNF, or any other additives for the growth of cells known to the skilled artisan. Other additives for the growth of cells include, but are not limited to, surfactant, Plasmanate®, and reducing agents such as N-acetyl-cysteine and 2-mercaptoethanol. Media can include RPMI 1640 (as noted herein), AIM V, DMEM, MEM, α-MEM, F-12, X-VIVO™ 10, X-VIVO™ 15 and X-VIVO™ 20, OpTmizer™, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). Immune cells e.g., T cells that have been exposed to varied stimulation times can exhibit different characteristics.

In some embodiments, the cells of the instant disclosure can be expanded by co-culturing with tissue or cells. The cells can also be expanded in vivo, for example in the subject's blood after administrating the cell into the subject.

### Compositions and Populations Comprising Engineered Immune Cells

In another aspect, the instant disclosure provides compositions (such as pharmaceutical compositions) comprising any of the cells of the instant disclosure. In some embodiments, the composition comprises a T cell comprising a polynucleotide encoding an antigen binding protein e.g., a CAR, and/or a CD70 binding protein. The cell is further engineered to express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level relative to comparable cells not engineered to functionally express a reduced or altered level of the corresponding protein, or comprise a population of cells that comprises an engineered immune cell e.g., T cell of the instant disclosure, and one or more pharmaceutically acceptable carriers or excipients. In another embodiment, the cell is not further engineered to express a reduced level of β2m. In another embodiment, the engineered immune cell further comprises a polynucleotide encoding a CD70 binding protein and/or functionally expresses a CD70 binding protein, as described herein.

In one embodiment, the engineered cell, such as an engineered immune cell, e.g., a CAR T cell, described herein comprises one or more genomic modifications that functionally impair or reduce expression of one or more targets described herein. In one embodiment, the one or more genomic modifications are at the genomic location of one or more genes or are elsewhere within the genome and not at the location of the one or more genes, such that the modifications functionally impair or reduce expression of the one or more genes.

In some embodiments, primary cells isolated from a donor are engineered as described herein to provide a population of cells of which a subpopulation (e.g., a proportion less than 100%, such as 10%, 20%, 30%, 40%, 50%, 60%, 70% 80% or 90%) of the resulting cells comprise all of the desired modifications. Such a resulting population, comprising a mixture of cells that comprise all of the modifications and cells that do not, can be used in the methods of treatment of the instant disclosure and to prepare the compositions of the instant disclosure. Alternatively, this population of cells (the “starting population”) can be manipulated by known methods e.g., cell sorting and/or expansion of cells that have the desired modifications, to provide a population of cells that is enriched for those cells comprising one or more of the desired modifications (e.g., enriched for cells that express the desired antigen binding protein and/or enriched for cells that express
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level relative to comparable cells not engineered with respect to expression level of the corresponding protein), that is, that comprises a higher percentage of such modified or engineered cells than did the starting population. The enriched population of cells may comprise a higher percentage of such modified or engineered cells than did the starting population. In another embodiment, the enriched population does not comprise cells that express β2m at a reduced level. In another embodiment, the enriched population comprises one or more engineered immune cells that further comprise a polynucleotide encoding a CD70 binding protein and/or functionally express a CD70 binding protein, as described herein.

The population enriched for the modified cells can then be used in the methods of treatment of the instant disclosure and to prepare the compositions of the instant disclosure, for example. In some embodiments, the enriched population of cells contains, or contains at least, for example, 20%, 30%, 40%, 50%, 60%, 70%, 80%, 90%, 95%, or 99% cells that have one or more of the modifications. In other embodiments, the proportion of cells of the enriched population of cells that comprise one or more of the modifications is at least 30% higher than the proportion of cells of the starting population of cells that comprise the desired modifications.

### Methods of Treating

In one aspect, the present disclosure provides methods of evading the immune response of a subject during a course of immune cell therapy. In one embodiment, the immune cell therapy comprises administering to the subject allogeneic immune cells, wherein the allogeneic immune cells are engineered immune cells. In other embodiments, the engineered immune cells are engineered to functionally express a reduced level of any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1; both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; (x) CD48, CD58, and ICAM-1; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   wherein the engineered immune cells (i) are less vulnerable to rejection via an alloreactive immune response of the subject, (ii) are less vulnerable to rejection via an alloreactive immune response of CD8+ and/or CD4+ T cells of the subject, and/or (iii) have improved capacity to coexist with the subject's immune cells, as compared to non-engineered immune cells. In another embodiment, the engineered immune cells are further characterized as having in vivo CAR T efficacy simultaneously with one or more of (i), (ii), and (iii). In another embodiment, the engineered immune cells further comprise a polynucleotide encoding a CD70 binding protein and/or functionally express a CD70 binding protein, as described herein.

Immune cells, e.g., engineered immune cells, such as the T cells obtained by the methods described above, or cell lines derived from such immune cells or T cells, can be used to treat a condition or a disorder in a subject, or can be used as a medicament. In some embodiments, such methods and/or medicaments can be used for treating a condition or a disorder such as for example a viral disease, a bacterial disease, a cancer, an inflammatory disease, an immune disease, or an aging-associated disease. In some embodiments, the cancer can be selected from the group consisting of gastric cancer, sarcoma, lymphoma (including Non-Hodgkin's lymphoma), leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, immune cells e.g., T cells according to the instant disclosure, or cell line derived from the immune cells e.g., engineered T cells, can be used in the manufacture of a medicament for treatment of a condition or a disorder in a subject in need thereof. In some embodiments, the condition or disorder can be, for example, a cancer, an autoimmune disorder, or an infection.

Also provided herein are methods for treating subjects. In some embodiments the method comprises administering or providing an immune cell e.g., an engineered T cell of the instant disclosure to a subject in need thereof. In some embodiments, the method comprises a step of administering the immune cells e.g., T cells of the instant disclosure, to a subject in need thereof.

In some embodiments, immune cells e.g., engineered T cells of the instant disclosure can undergo robust in vivo cell expansion and can persist for an extended amount of time. Methods of treatment of the instant disclosure can be ameliorating, curative or prophylactic. The method of the instant disclosure can be either part of an autologous immunotherapy or part of an allogenic immunotherapy treatment. The instant disclosure is particularly suitable for allogeneic immunotherapy. Immune cells e.g., engineered T cells provided by a donor, can be transformed into non-alloreactive cells using standard protocols and reproduced as needed, thereby producing e.g., CAR-T cells which can be administered to one or several subjects. Such CAR-T cell therapy can be made available as an allogeneic ALLO CAR T™ therapeutic product.

In another aspect, the instant disclosure provides a method of inhibiting tumor growth or progression in a subject who has a tumor, comprising administering to the subject an effective amount of engineered immune cells e.g., engineered T cells as described herein. In another aspect, the present disclosure provides a method of inhibiting or preventing metastasis of cancer cells in a subject, comprising administering to the subject in need thereof an effective amount of engineered immune cells e.g., engineered T cells as described herein. In another aspect, the instant disclosure provides a method of inducing tumor regression in a subject who has a tumor, comprising administering to the subject an effective amount of engineered immune cells, e.g., engineered T cells as described herein.

In some embodiments, the immune cells, e.g., T cells provided herein can be administered parenterally to a subject. In some embodiments, the subject is a human.

In some embodiments, the method can further comprise administering an effective amount of a second therapeutic agent. In some embodiments, the second therapeutic agent is, for example, crizotinib, palbociclib, an anti-CTLA4 antibody, an anti-4-1 BB antibody, a PD-1 antibody, or a PD-L1 antibody.

Also provided is the use of any of the immune cells e.g., T cells provided herein in the manufacture of a medicament for the treatment of cancer or for inhibiting tumor growth or progression in a subject in need thereof.

In certain embodiments, the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   or the functional expression level of any other gene that is knocked down or knocked out according to the present disclosure, in an engineered immune cell of the instant disclosure is decreased by or by at least about 25%, 30%, 35%, 40%, 45%, 50%, 55%, 60%, 65%, 70%, 75%, 80%, 85%, 90%, 95%, 99%, or 100% relative to the corresponding expression level in a comparable but not so genetically-modified engineered immune cell.
   In one other embodiment, the functional expression level of β2m in an engineered immune cell of the instant disclosure is not decreased. In another embodiment, the engineered immune cell further comprises a polynucleotide encoding a CD70 binding protein and/or functionally expresses a CD70 binding protein, as described herein.

Expression levels can be determined by any known method, such as FACS or MACs. In some embodiments, the engineered immune cell disclosed herein functionally expresses
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   or any other gene that is knocked down or knocked out according to the present disclosure, at a level not greater than 75%, not greater than 50%, not greater than 25%, not greater than 10% or at a level of 0% of the expression level in non-engineered immune cells that otherwise are the same as the engineered immune cells, e.g., comprise the same components as the engineered immune cells. In another embodiments, the engineered immune cell does not functionally express β2m at a reduced level relative to the expression level of β2m in non-engineered immune cells that otherwise are the same as the engineered immune cell, e.g., comprise the same components as the engineered immune cell. In another embodiment, the engineered immune cell further comprises a polynucleotide encoding a CD70 binding protein and/or functionally expresses a CD70 binding protein, as described herein.

In some embodiments, both alleles of one gene are knocked out, so that gene's expression level in the engineered immune cell disclosed herein is 0% of that of a corresponding non-engineered cell. In some embodiments, one of the two alleles of a gene is knocked out, so that gene's expression level in the engineered immune cell disclosed herein is 50% or about 50% (e.g., if a compensatory mechanism causes greater than normal expression of the remaining allele) of that of a corresponding non-engineered cell. Intermediate levels of expression can be observed if, for example, expression is reduced by some means other than knock-out, as described herein.

In some embodiments, the expression level of one or more of CD48, CD58, ICAM-1, TAP2, NLRC5, β2m, TRAC, RFX5, RFXAP, CIITA and RFXANK, or of any other gene the expression level of which is manipulated according to the present disclosure, in the engineered cells of the present disclosure can be measured directly by assaying the cells for gene products and their properties using standard techniques known to those of skill in the art (e.g., RT-qPCR, nucleic acid sequencing, antibody staining, or some combination of techniques). In some embodiments, the functional expression level of one or more of TAP2, NLRC5, β2m, TRAC, CIITA, RFX5, RFXAP and RFXANK is measured by determining the surface expression level of one or more HLA proteins, such as an HLA-A or HLA-B protein, or of beta2 microglobulin (B2M), or of both B2M and one or more HLA proteins on the surface of the engineered immune cell by standard techniques known in the art, e.g., flow cytometry. In various embodiments of the present disclosure, the functional expression level of any one or more of CD48, CD58, and ICAM-1 is measured by determining the surface expression level of each cell surface protein, such as one or more of a CD48, a CD58 or an ICAM-1 protein on the surface of the engineered immune cell, or is measured by flow cytometry. These measurements can be compared to corresponding measurements made on comparable cells that have not been engineered to reduce the corresponding functional expression level. In a population of cells that comprises an engineered cell e.g., engineered immune cell of the invention, a pooled sample of the material being measured, e.g., RNA or protein or cells, will reflect the fact that some of the cells do not express the gene of interest, having had both alleles knocked out, for example, some of the cells express the gene of interest at 50% or about 50%, having had only one allele knocked out, and, if the population comprises non-engineered cells, that some of the cells express a normal level of the gene of interest.

The functional expression level of one or more of CD48, CD58, ICAM-1, TAP2, NLRC5, β2m, TRAC, CIITA, RFX5, RFXAP and RFXANK expression in engineered immune cells of the present disclosure can also be assayed, for example, by measuring the degree to which the engineered immune cells survive in the presence of effector cells e.g., T cells or NK cells, in comparison to the degree to which non-engineered, but otherwise comparable e.g., identical, immune cells survive under the same conditions. See, e.g., FIG. 1D-1E and Example 1.

In some embodiments, administering an engineered immune cell e.g., engineered T cell as disclosed herein, or administering a population of cells comprising such engineered immune cells e.g., engineered T cells, reduces host rejection of the administered cell or population of cells relative to a comparable but non-engineered cell or comparable population that does not comprise such engineered cells. In some embodiments, administering an engineered immune cell, e.g., engineered T cell of the instant disclosure, comprising an antigen binding protein e.g., a CAR, and/or a CD70 binding protein and in which the expression level e.g., functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   is reduced, or administering a population of cells comprising such engineered immune cells, e.g., engineered T cells, wherein the administering reduces host rejection of the administered cell or population of cells relative to a comparable but non-engineered cell or population that does not comprise such engineered cells. In another embodiment, the expression level, e.g., functional expression level of β2m is not reduced. In another embodiment, the engineered immune cells further comprise a polynucleotide encoding a CD70 binding protein and/or functionally express a CD70 binding protein, as described herein.

For example, such administration reduces host rejection by between 1% and 99%, e.g., between 5% and 95%, between 10% and 90%, between 50% and 90%, e.g., by 10%, 20%, 30%, 40%, 50%, 60%, 70%, 80%, or 90% as compared to host rejection of cells that are the same but which are not engineered to express the corresponding protein at a reduced level. In some embodiments, host rejection is reduced by over 90%.

In some embodiments, administering an engineered immune cell e.g., T cell of the instant disclosure comprising an antigen binding protein e.g., a CAR, and/or a CD70 binding protein, and in which the functional expression level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   is reduced, or administering a population of cells comprising such immune cells, e.g., T cells, wherein the administering enhances or improves the persistence and/or increases the persistence of the cells as compared to the persistence of cells that are the same but which are not engineered to express a reduced level of the corresponding protein. In another embodiment, the functional expression level of β2m is not reduced. In another embodiment, the immune cells further comprises a polynucleotide encoding a CD70 binding protein and/or functionally expresses a CD70 binding protein, as described herein.

In some embodiments, persistence is increased by, for example, between 1 and 7 days, by between 1 and 12 weeks (e.g., between 1 and 4 weeks, 4 and 8 weeks, or 8 and 12 weeks), or by between 1 and 12 months, or by a specific length of time that falls within these ranges. In some embodiments, the difference in persistence is measured by comparing the half-life of the administered cells in the population or composition, wherein, for example, the half-life is increased by, for example, between 1 and 7 days, by between 1 and 12 weeks (e.g., between 1 and 4 weeks, 4 and 8 weeks, or 8 and 12 weeks), or by between 1 and 12 months, or by a specific length of time that falls within these ranges. In some embodiments, the difference in persistence is measured by comparing the length of time that the administered cells can be detected after administration. In some embodiments, the improvement in persistence is measured in vitro by comparing the survival of engineered and non-engineered cells in the presence of, for example, immune cells such as T cells or NK cells, e.g., at about 72 hours, 5 days, 7 days or 13 days after mixing. In some embodiments, in such an in vitro assay, between about 1.5 and 10 times as many engineered cells survive as do cells that are not engineered at the time of measurement. In some embodiments, in such an in vitro assay, between about 1.5 and 10 times as many engineered immune cells survive as do immune cells that are not engineered at the time of measurement. The degree of improved persistence or survival of the engineered immune cells described herein depends in part on the degree to which the functional level of expression of one or more targets is reduced and additionally but optionally the level of expression of CD70 in the co-incubated (e.g. “attacking” or host) immune cells.

In some embodiments, reduction in host rejection and/or increases in persistence of administered cells as disclosed herein are determined by any of a variety of techniques known to the person of ordinary skill in the art. In some embodiments, any one or a combination of the following is use: flow cytometry, PCR, e.g., quantitative PCR, and ex vivo coincubation with patient tumor material or with a model tumor cell line expressing the antigen targeted by the CAR-T cell. In some embodiments, qPCR is used to assess the number of CAR T cells that have and do not have the knock-out of interest in order to determine the extent to which the knock-out provides a survival advantage.

In some embodiments, the treatment can be in combination with one or more therapies against cancer selected from the group of antibodies therapy, chemotherapy, cytokines therapy, dendritic cell therapy, gene therapy, hormone therapy, laser light therapy and radiation therapy.

In some embodiments, treatment can be administered to subjects undergoing an immunosuppressive treatment. Indeed, the instant disclosure can rely on cells or a population of cells which have been made resistant to at least one immunosuppressive agent due to the inactivation of a gene encoding a receptor for such immunosuppressive agent. In this aspect, the immunosuppressive treatment can help the selection and expansion of the T cells according to the instant disclosure within the subject.

The administration of the cells or population of cells according to the instant disclosure can be carried out in any convenient manner, including by aerosol inhalation, injection, ingestion, transfusion, implantation or transplantation. The compositions described herein can be administered to a subject subcutaneously, intradermally, intratumorally, intranodally, intramedullary, intramuscularly, by intravenous or intralymphatic injection, or intraperitoneally. In one embodiment, the cell compositions of the instant disclosure are administered by intravenous injection.

In some embodiments, the administration of the cells or population of cells according to the instant disclosure can comprise administration of, for example, from about 10³ or 10⁴ to about 10⁹ cells per kg body weight including all integer values of cell numbers within those ranges. In some embodiments the administration of the cells or population of cells can comprise administration of about 10⁵ to about 10⁶ cells per kg body weight including all integer values of cell numbers within those range, or administration of between 0.1×10⁶ and 5×10⁶ engineered immune cells of the invention per kg body weight, or a total of between 0.1×10⁸ and 5×10⁸ engineered immune cells. The cells or population of cells can be administered in one or more doses. In some embodiments, an effective amount of cells can be administered as a single dose. In some embodiments, an effective amount of cells can be administered as more than one dose over a period time. Timing of administration is within the judgment of the managing physician and depends on the clinical condition of the subject. The cells or population of cells can be obtained from any source, such as a blood bank or a donor. While individual needs vary, determination of optimal ranges of effective amounts of a given cell type for a particular disease or conditions is within the skill of the art. An effective amount means an amount which provides a therapeutic or prophylactic benefit. The dosage administered will be dependent upon the age, health and weight of the recipient, the kind of concurrent treatment, if any, the frequency of treatment and the nature of the effect desired. In some embodiments, an effective amount of cells or composition comprising those cells are administered parenterally. In some embodiments, administration can be an intravenous administration. In some embodiments, administration can be directly done by injection within a tumor.

In some embodiments of the instant disclosure, cells are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, BCMA specific CAR-T cells are administered to a subject in conjunction with one or more of the following: an anti-PD-1 antibody (e.g., nivolumab, pembrolizumab), an anti-PD-L1 antibody (e.g., avelumab, atezolizumab, or durvalumab), an anti-OX40 antibody, an anti-4-1 BB antibody (e.g., Utolimumab), an anti-MCSF antibody, an anti-GITR antibody, and/or an anti-TIGIT antibody. In further embodiments, the immune cells, e.g., T cells, of the instant disclosure can be used in combination with chemotherapy, radiation, immunosuppressive agents, such as cyclosporin, azathioprine, methotrexate, mycophenolate, and FK506, antibodies, or other immunoablative agents such as CAMPATH (alemtuzumab), anti-CD3 antibodies or other antibody therapies, cytoxan, fludarabine, cyclophosphamide, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. Immunology. 1991 July; 73(3): 316-321; Liu, Albers et al. Biochemistry 1992 Apr. 28; 31(16):3896-901; Bierer, Hollander et al. Curr Opin Immunol. 1993 October; 5(5):763-73). In one embodiment, the immune cells, e.g., T cells, of the instant disclosure can be administered to a subject who has previously been administered an immunosuppressive agent, wherein the immunosuppressive agent lymphodepleted the subject thereby allowing engraftment of the immune cells. However, overtime the subject's immune system will reconstitute and recover from the lymphodepletion (see e.g., Tees et al. Safety and PK/PD of ALLO-647, an anti-CD52 antibody, with fludarabine (Flu)/cyclophosphamide (Cy) for lymphodepletion in the setting of allogeneic CAR-T cell therapy. J. Clin. Oncology, vol. 39, Issue 15_suppl 2021). As such, the engineered immune cells of the instant disclosure provide further protection from the subject's immune system at different times during the treatment regimen. In one embodiment, the engineered immune cells coexist with and actively engage the subject's immune system while avoiding rejection.

In a further embodiment, the cell compositions of the instant disclosure are administered to a subject in conjunction with (e.g., before, simultaneously or following) bone marrow transplantation, T cell ablative therapy using either chemotherapy agents such as fludarabine, external-beam radiation therapy (XRT), cyclophosphamide, or antibodies such as CAMPATH. In some embodiments, the cell compositions of the instant disclosure are administered following B-cell ablative therapy such as agents that react with CD20, e.g., Rituxan. For example, in one embodiment, subjects can undergo standard treatment with high dose chemotherapy followed by peripheral blood stem cell transplantation. In certain embodiments, following the transplant, subjects receive an infusion of expanded immune cells of the instant disclosure. In some embodiments, expanded cells are administered before or following surgery.

### Kits

The instant disclosure also provides kits for use in the instant methods. Kits of the instant disclosure include one or more containers comprising a composition of the instant disclosure or an immune cell, e.g., a T cell of the instant disclosure or a population of cells comprising an immune cell, e.g., an engineered T cell of the instant disclosure. In various embodiments, the immune cell, e.g., T cell comprises one or more polynucleotide(s) encoding an antigen binding protein, e.g., a CAR as described herein, and further is engineered to express a reduced level of one or more of CD48, CD58, ICAM-1, TAP2, NLRC5, β2m, TRAC, RFX5, RFXAP, CIITA and RFXANK as described herein. In another embodiment, the immune cell is further engineered to express a reduced level of
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   as described herein. In another embodiment, the immune cell is not further engineered to express a reduced level of β2m.

The kit further comprises instructions for use in accordance with any of the methods of the instant disclosure described herein. Generally, these instructions comprise a description of administration of the composition, immune cell, e.g., a T cell or population of cells for the above described therapeutic treatments.

The instructions relating to the use of the kit components generally include information as to dosage, dosing schedule, and route of administration for the intended treatment. The containers can be unit doses, bulk packages (e.g., multi-dose packages) or subunit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of the present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition is an immune cell e.g., T cell according to the instant disclosure. The container can further comprise a second pharmaceutically active agent.

Kits can optionally provide additional components such as buffers and interpretive information. Normally, the kit comprises a container and a label or package insert(s) on or associated with the container.

### Methods of Sorting and Depletion

In some embodiments, provided are methods for in vitro sorting of a population of immune cells, wherein a subset of the population of immune cells comprises immune cells engineered as described herein to express
any one or more of CD48, CD58, ICAM-1, TAP2, NLRC5, β2m, TRAC, CIITA, RFX5, RFXAP and RFXANK;
only one of CD48, CD58 and ICAM-1;
(i) one or both of NLRC5 and RFX5 and (ii) one or more of CD58, CD48, and ICAM-1;
both NLRC5 and CD58, both RFX5 and CD58, both RFX5 and CD48, both NLRC5 and CD48, both RFX5 and ICAM-1, both NLRC5 and ICAM-1, both CD58 and ICAM-1, both CD58 and CD48, or both CD48 and ICAM-1;
(i) CD58, NLRC5, and RFX5; (ii) CD48, NLRC5, and RFX5; (iii) ICAM-1, NLRC5, and RFX5; (iv) CD58, ICAM-1, and RFX5; (v) CD48, ICAM-1, and RFX5; (vi) CD58, CD48, and RFX5; (vii) CD58, ICAM-1, and NLRC5; (viii) CD48, ICAM-1, and NLRC5; or (ix) CD58, CD48, and NLRC5; or
(i) CD58, ICAM-1, RFX5, and NLRC5; (ii) CD48, ICAM-1, RFX5, NLRC5; (iii) CD48, CD58, RFX5, and NLRC5; (iv) CD48, CD58, ICAM-1, and RFX5; (v) CD48, CD58, ICAM-1, and NLRC5; (vi) β2m, CD58, CD48, and ICAM-1; or (vii) CD48, CD58, ICAM-1, RFX5, and NLRC5,
   at a reduced level and/or express an antigen binding protein, e.g., a CAR.

In various embodiments the method comprises contacting the population of immune cells with a monoclonal antibody specific for an epitope (e.g., a mimotope such as those provided in US2018/0002435) unique to the engineered cell, e.g., an epitope of the antigen binding protein or a mimotope incorporated into the antigen binding protein, and selecting the immune cells that bind to the monoclonal antibody to obtain a population of cells enriched in engineered immune cells that express the antigen binding protein.

In some embodiments, the monoclonal antibody specific for the epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the antigen binding protein e.g., CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10.

In some embodiments, the population of CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30% or 50%. In some embodiments, the immune cells are T-cells.

The CAR-expressing immune cells to be administered to the recipient can be enriched in vitro from the source population. Methods of expanding source populations can include selecting cells that express an antigen such as CD34 antigen, using combinations of density centrifugation, immuno-magnetic bead purification, affinity chromatography, and fluorescent activated cell sorting.

Flow cytometry can be used to quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometry analysis involves two primary steps: 1) labeling selected cell types with one or more labeled markers, and 2) determining the number of labeled cells relative to the total number of cells in the population. In some embodiments, the method of labeling cell types includes binding labeled antibodies to markers expressed by the specific cell type. The antibodies can be either directly labeled with a fluorescent compound or indirectly labeled using, for example, a fluorescent-labeled second antibody which recognizes the first antibody.

In some embodiments, the method used for sorting T cells expressing CAR is the Magnetic-Activated Cell Sorting (MACS). Magnetic-activated cell sorting (MACS) is a method for separation of various cell populations depending on their surface antigens (CD molecules) by using superparamagnetic nanoparticles and columns. MACS can be used to obtain a pure cell population. Cells in a single-cell suspension can be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

A detailed protocol for the purification of a specific cell population such as T-cells can be found in Basu S et al. (2010). (Basu S, Campbell H M, Dittel B N, Ray A. Purification of specific cell population by fluorescence activated cell sorting (FACS). J Vis Exp. (41): 1546).

TABLE 1
Other exemplary sequences

| | | SEQ |
|---|---|---|
| | | ID |
| Domain | Amino Acid Sequence | NO: |
| CD3zeta zeta | LDKRRGRDPEMGGKPRRKNP | 1 |
| concatenated | QEGLYNELQKDKMRVKFSRS | |
| cytoplasmic | ADAPAYQQGQNQLYNELNLG | |
| domain | RREEYDVAEAYSEIGMKGER | |
| | RRGKGHDGLYQGLSTATKDT | |
| | YDALHMQALPPRGRVKFSRS | |
| | ADAPAYQQGQNQLYNELNLG | |
| | RREEYDVLDKRRGRDPEMGG | |
| | KPRRKNPQEGLYNELQKDKM | |
| | AEAYSEIGMKGERRRGKGHD | |
| | GLYQGLSTATKDTYDALHMQ | |
| | ALPPR | |
| CD8α signal peptide | MALPVTALLLPLALLLHAAR | 2 |
| | P | |
| FcγRIIIα hinge | GLAVSTISSFFPPGYQ | 3 |
| CD8α hinge | TTTPAPRPPTPAPTIASQPL | 4 |
| | SLRPEACRPAAGGAVHTRGL | |
| | DFACD | |
| IgG1 hinge | EPKSPDKTHTCPPCPAPPVA | 5 |
| | GPSVFLFPPKPKDTLMIART | |
| | PEVTCVVVDVSHEDPEVKFN | |
| | WYVDGVEVHNAKTKPREEQY | |
| | NSTYRVVSVLTVLHQDWLNG | |
| | KEYKCKVSNKALPAPIEKTI | |
| | SKAKGQPREPQVYTLPPSRD | |
| | ELTKNQVSLTCLVKGFYPSD | |
| | IAVEWESNGQPENNYKTTPP | |
| | VLDSDGSFFLYSKLTVDKSR | |
| | WQQGNVFSCSVMHEALHNHY | |
| | TQKSLSLSPGK | |
| CD8α | IYIWAPLAGTCGVLLLSLVI | 6 |
| transmembrane | TLYC | |
| (TM) | | |
| domain | | |
| 41BB intracellular | KRGRKKLLYIFKQPFMRPVQ | 7 |
| signaling domain | TTQEEDGCSCRFPEEEEGGC | |
| (ISD) | EL | |
| CD3ζ intracellular | RVKFSRSADAPAYQQGQNQL | 8 |
| signaling | YNELNLGRREEYDVLDKRRG | |
| domain (ISD) | RDPEMGGKPRRKNPQEGLYN | |
| | ELQKDKMAEAYSEIGMKGER | |
| | RRGKGHDGLYQGLSTATKDT | |
| | YDALHMQALPPR | |
| FcϵRI α-TM-IC | FFIPLLVVILFAVDTGLFIS | 9 |
| (FcϵRI α | TQQQVTFLLKIKRTRKGFRL | |
| chain | LNPHPKPNPKNN | |
| transmembrane | | |
| and | | |
| intracellular | | |
| domain) | | |
| FcϵRIβ-ΔITAM | MDTESNRRANLALPQEPSSV | 10 |
| (FcϵRI β | PAFEVLEISPQEVSSGRLLK | |
| chain | SASSPPLHTWLTVLKKEQEF | |
| without ITAM) | LGVTQILTAMICLCFGTVVC | |
| | SVLDISHIEGDIFSSFKAGY | |
| | PFWGAIFFSISGMLSIISER | |
| | RNATYLVRGSLGANTASSIA | |
| | GGTGITILIINLKKSLAYIH | |
| | IHSCQKFFETKCFMASFSTE | |
| | IVVMMLFLTILGLGSAVSLT | |
| | ICGAGEELKGNKVPE | |
| CD3 delta | GHETGRLSGAADTQALLRND | 11 |
| cytoplasmic | QVYQPLRDRDDAQYSHLGGN | |
| domain | WARNK | |
| CD28-IC (CD28 co- | RSKRSRGGHSDYMNMTPRRP | 12 |
| stimulatory domain) | GPTRKHYQPYAPPRDFAAYR | |
| | S | |
| FcϵRIγ-SP (signal | MIPAVVLLLLLLVEQAAA | 13 |
| peptide) | | |
| FcϵRI γ-ΔITAM | LGEPQLCYILDAILFLYGIV | 14 |
| (FcϵRI γ | LTLLYCRLKIQVRKAAITSY | |
| chain without | EKS | |
| ITAM) | | |
| GSG-P2A (GSG- | GSGATNFSLLKQAGDVEENP | 15 |
| P2A ribosomal | GP | |
| skip | | |
| polypeptide) | | |
| GSG-T2A (GSG- | GSGEGRGSLLTCGDVEENPG | 16 |
| T2A ribosomal | P | |
| skip | | |
| polypeptide) | | |
| CD3 epsilon | KNRKAKAKPVTRGAGAGGRQ | 17 |
| cytoplasmic | RGQNKERPPPVPNPDYEPIR | |
| domain | KGQRDLYSGLNQRRI | |
| CD3 gamma | GQDGVRQSRASDKQTLLPND | 18 |
| cytoplasmic | QLYQPLKDREDDQYSHLQGN | |
| domain | QLRRN | |
| CD3 zeta | APAYQQGQNQLYNELNLGRR | 19 |
| ITAM zeta1 | EEYDVLDKR | |
| CD3 zeta | PRRKNPQEGLYNELQKDKMA | 20 |
| ITAM zeta2 | EAYSEIGM | |
| CD3 zeta | ERRRGKGHDGLYQGLSTATK | 21 |
| ITAM zeta3 | DTYDALHMQ | |
| CD3 delta | DTQALLRNDQVYQPLRDRDD | 22 |
| ITAM delta | AQYSHLGGN | |
| CD3 epsilon ITAM | ERPPPVPNPDYEPIRKGQRD | 23 |
| epsilon | LYSGLNQR | |
| CD3 gamma ITAM | DKQTLLPNDQLYQPLKDRED | 24 |
| gamma | DQYSHLQGN | |
| CD3 zeta | RVKFSRSADDTQALLRNDQV | 25 |
| variant | YQPLRDRDDAQYSHLGGNRG | |
| (dzz) | RDPEMGGKPRRKNPQEGLYN | |
| | ELQKDKMAEAYSEIGMKGER | |
| | RRGKGHDGLYQGLSTATKDT | |
| | YDALHMQALPPR | |
| CD3 zeta | RVKFSRSADERPPPVPNPDY | 26 |
| variant | EPIRKGQRDLYSGLNQRRGR | |
| (ezz) | DPEMGGKPRRKNPQEGLYNE | |
| | LQKDKMAEAYSEIGMKGERR | |
| | RGKGHDGLYQGLSTATKDTY | |
| | DALHMQALPPR | |
| CD3 zeta | RVKFSRSADDKQTLLPNDQL | 27 |
| variant | YQPLKDREDDQYSHLQGNRG | |
| (gzz) | RDPEMGGKPRRKNPQEGLYN | |
| | ELQKDKMAEAYSEIGMKGER | |
| | RRGKGHDGLYQGLSTATKDT | |
| | YDALHMQALPPR | |
| CD3 zeta | RVKFSRSADDTQALLRNDQV | 28 |
| variant | YQPLRDRDDAQYSHLGGNRG | |
| (deg) | RDPEMGGKERPPPVPNPDYE | |
| | PIRKGQRDLYSGLNQRKGDK | |
| | QTLLPNDQLYQPLKDREDDQ | |
| | YSHLQGNALPPR | |
| CD3 zeta | RVKFSRSADAPAYQQGQNQL | 29 |
| zeta | YNELNLGRREEYDVLDKRRG | |
| (zdzezg) | RDPEMGGKDTQALLRNDQVY | |
| | QPLRDRDDAQYSHLGGNKGP | |
| | RRKNPQEGLYNELQKDKMAE | |
| | AYSEIGMALPPRGRVKFSRS | |
| | ADERPPPVPNPDYEPIRKGQ | |
| | RDLYSGLNQRRGRDPEMGGK | |
| | ERRRGKGHDGLYQGLSTATK | |
| | DTYDALHMQKGDKQTLLPND | |
| | QLYQPLKDREDDQYSHLQGN | |
| | ALPPR | |
| CD3 zeta | RVKFSRSADDTQALLRNDQV | 30 |
| zeta | YQPLRDRDDAQYSHLGGNRG | |
| (dzezgz) | RDPEMGGKAPAYQQGQNQLY | |
| | NELNLGRREEYDVLDKRKGE | |
| | RPPPVPNPDYEPIRKGQRDL | |
| | YSGLNQRALPPRGRVKFSRS | |
| | ADPRRKNPQEGLYNELQKDK | |
| | MAEAYSEIGMRGRDPEMGGK | |
| | DKQTLLPNDQLYQPLKDRED | |
| | DQYSHLQGNKGERRRGKGHD | |
| | GLYQGLSTATKDTYDALHMQ | |
| | ALPPR | |
| CD3 zeta | RVKFSRSADAPAYQQGQNQL | 31 |
| zeta | YNELNLGRREEYDVLDKRRG | |
| (zzzdeg) | RDPEMGGKPRRKNPQEGLYN | |
| | ELQKDKMAEAYSEIGMKGER | |
| | RRGKGHDGLYQGLSTATKDT | |
| | YDALHMQALPPRGRVKFSRS | |
| | ADDTQALLRNDQVYQPLRDR | |
| | DDAQYSHLGGNRGRDPEMGG | |
| | KERPPPVPNPDYEPIRKGQR | |
| | DLYSGLNQRKGDKQTLLPND | |
| | QLYQPLKDREDDQYSHLQGN | |
| | ALPPR | |
| CD3 zeta | RVKFSRSADDTQALLRNDQV | 32 |
| zeta | YQPLRDRDDAQYSHLGGNRG | |
| (degzzz) | RDPEMGGKERPPPVPNPDYE | |
| | PIRKGQRDLYSGLNQRKGDK | |
| | QTLLPNDQLYQPLKDREDDQ | |
| | YSH | |
| | LQGNALPPRGRVKFSRSADA | |
| | PAYQQGQNQLYNELNLGRRE | |
| | EYDVLDKRRGRDPEMGGKPR | |
| | RKNPQEGLYNELQKDKMAEA | |
| | YSEIGMKGERRRGKGHDGLY | |
| | QGLSTATKDTYDALHMQALP | |
| | PR | |
| CD3 zeta | RVKFSRSADAPAYQQGQNQL | 33 |
| YA | YAELNLGRREEYDVLDKRRG | |
| | RDPEMGGKPRRKNPQEGLYN | |
| | ELQKDKMAEAYSEIGMKGER | |
| | RRGKGHDGLYQGLSTATKDT | |
| | YDALHMQALPPR | |
| CD3 zeta | RVKFSRSADAPAYQQGQNQL | 34 |
| YAYAYA | YAELNLGRREEYDVLDKRRG | |
| | RDPEMGGKPRRKNPQEGLYA | |
| | ELQKDKMAEAYSEIGMKGER | |
| | RRGKGHDGLYAGLSTATKDT | |
| | YDALHMQALPPR | |
| CD3 zeta | RVKFSRSADAPAYQQGQNQL | 35 |
| YAtrunc | YAELNLGRREEYDVLDKRAL | |
| | PPR | |
| CD3 zeta | RVKFSRSADAPAYQQGQNQL | 36 |
| zeta | YAELNLGRREEYDVLDKRRG | |
| (zdzezg- | RDPEMGGKDTQALLRNDQVY | |
| 6xYA) | APLRDRDDAQYSHLGGNKGP | |
| | RRKNPQEGLYAELQKDKMAE | |
| | AYSEIGMALPPRGRVKFSRS | |
| | ADERPPPVPNPDYAPIRKGQ | |
| | RDLYSGLNQRRGRDPEMGGK | |
| | ERRRGKGHDGLYAGLSTATK | |
| | DTYDALHMQKGDKQTLLPND | |
| | QLYAPLKDREDDQYSHLQGN | |
| | ALPPR | |
| CD3 zeta | RVKFSRSADDTQALLRNDQV | 37 |
| zeta | YAPLRDRDDAQYSHLGGNRG | |
| (dzezgz- | RDPEMGGKAPAYQQGQNQLY | |
| 6xYA) | AELNLGRREEYDVLDKRKGE | |
| | RPPPVPNPDYAPIRKGQRDL | |
| | YSGLNQRALPPRGRVKFSRS | |
| | ADPRRKNPQEGLYAELQKDK | |
| | MAEAYSEIGMRGRDPEMGGK | |
| | DKQTLLPNDQLYAPLKDRED | |
| | DQYSHLQGNKGERRRGKGHD | |
| | GLYAGLSTATKDTYDALHMQ | |
| | ALPPR | |
| CD3 zeta | APAYQQGQNQLYAELNLGRR | 38 |
| ITAM zeta1 | EEYDVLDKR | |
| YA | | |
| CD3 zeta | PRRKNPQEGLYAELQKDKMA | 39 |
| ITAM zeta2 | EAYSEIGM | |
| YA | | |
| CD3 zeta | ERRRGKGHDGLYAGLSTATK | 40 |
| ITAM zeta3 | DTYDALHMQ | |
| YA | | |
| CD3 delta | DTQALLRNDQVYAPLRDRDD | 41 |
| ITAM delta | AQYSHLGGN | |
| YA | | |
| CD3 epsilon ITAM | ERPPPVPNPDYAPIRKGQRD | 42 |
| epsilon YA | LYSGLNQR | |
| CD3 gamma ITAM | DKQTLLPNDQLYAPLKDRED | 43 |
| gamma YA | DQYSHLQGN | |
| CD3 zeta | APAYQQGQNQLYAELNLGRR | 44 |
| ITAM zeta1 | EEYDVLDKR | |
| YA | | |
| CD3 zeta | PRRKNPQEGLYAELQKDKMA | 45 |
| ITAM zeta2 | EAYSEIGM | |
| YA | | |
| CD3 zeta | ERRRGKGHDGLYAGLSTATK | 46 |
| ITAM zeta3 | DTYDALHMQ | |
| YA | | |
| CD3 delta ITAM delta | DTQALLRNDQVYAPLRDRDD | 47 |
| YA | AQYSHLGGN | |
| CD3 epsilon ITAM | ERPPPVPNPDYAPIRKGQRD | 48 |
| epsilon YA | LYSGLNQR | |
| CD3 gamma ITAM | DKQTLLPNDQLYAPLKDRED | 49 |
| gamma YA | DQYSHLQGN | |
| CD3 zeta | APAYQQGQNQLYAELNLGRR | 50 |
| ITAM zeta1 | EEYDVLDKR | |
| YA | | |
| CD3 zeta | PRRKNPQEGLYAELQKDKMA | 51 |
| ITAM zeta2 | EAYSEIGM | |
| YA | | |
| CD3 zeta | ERRRGKGHDGLYAGLSTATK | 52 |
| ITAM zeta | DTYDALHMQ | |
| 3 | | |
| YA | | |
| CD3 delta | DTQALLRNDQVYAPLRDRDD | 53 |
| ITAM delta | AQYSHLGGN | |
| YA | | |
| CD3 epsilon ITAM | ERPPPVPNPDYAPIRKGQRD | 54 |
| epsilon YA | LYSGLNQR | |
| CD3 gamma ITAM | DKQTLLPNDQLYAPLKDRED | 55 |
| gamma YA | DQYSHLQGN | |
| CD3 zeta | APAYQQGQNQLYAELNLGRR | 56 |
| ITAM zeta1 | EEYDVLDKR | |
| YA | | |
| CD28 | RSKRSRLLHSDYMNMTPRRP | 57 |
| intracellular | GPTRKHYQPYAPPRDFAAYR | |
| | S | |
| CD28.YMFM | RSKRSRLLHSDYMFMTPRRP | 58 |
| intracellular | GPTRKHYQPYAPPRDFAAYR | |
| | S | |
| CD28.AYAA | RSKRSRLLHSDYMNMTPRRP | 59 |
| intracellular | GPTRKHYQAYAAPRDFAAYR | |
| | S | |
| CD28 variant | RSKRSRLLHSDFMNMTARRA | 60 |
| | GPTRKHYQPYAPPRDFAAYR | |
| | S | |
| CD2 | KRKKQRSRRNDEELETRAHR | 61 |
| intracellular | VATEERGRKPHQIPASTPQN | |
| (full) | PATSQHPPPPPGHRSQAPSH | |
| | RPPPPGHRVQHQPQKRPPAP | |
| | SGTQVHQQKGPPLPRPRVQP | |
| | KPPHGAAENSLSPSSN | |
| CD2 | KRKKQTPQNPATSQHPPPPP | 62 |
| intracellular | GHRSQAPSHRPPPPGHRVQH | |
| (truncated) | QPQKRPPAPSGTQVHQQKGP | |
| | PLPRPRVQPKPPHGAAENSL | |
| | SPSSN | |
| OX40 | ALYLLRRDQRLPPDAHKPPG | 63 |
| intracellular | GGSFRTPIQEEQADAHSTLA | |
| | KI | |
| CD3 zeta | RVKFSRSADAPAYQQGQNQL | 64 |
| variant | YNELNLGRREEYDVLDKRRG | |
| (1XX) | RDPEMGGKPRRKNPQEGLFN | |
| | ELQKDKMAEAFSEIGMKGER | |
| | RRGKGHDGLFQGLSTATKDT | |
| | FDALHMQALPPR | |
| CD3 zeta | RVKFSRSADAPAYQQGQNQL | 65 |
| variant | FNELNLGRREEFDVLDKRRG | |
| (XX3) | RDPEMGGKPRRKNPQEGLFN | |
| | ELQKDKMAEAFSEIGMKGER | |
| | RRGKGHDGLYQGLSTATKDT | |
| | YDALHMQALPPR | |
| CD27 | QRRKYRSNKGESPVEPAEPC | 66 |
| intracellular | HYSCPREEEGSTIPIQEDYR | |
| | KPEPACSP | |
| CD3 zeta | RVKFSRSADAPAYQQGQNQL | 67 |
| variant | FNELNLGRREEFDVLDKRRG | |
| (X2X) | RDPEMGGKPRRKNPQEGLYN | |
| | ELQKDKMAEAYSEIGMKGER | |
| | RRGKGHDGLFQGLSTATKDT | |
| | FDALHMQALPPR | |
| CD3 zeta | RVKFSRSADERRRGKGHDGL | 68 |
| variant | YQGLSTATKDTYDALHMQRG | |
| (delta12) | RDPEMGGK | |
| CD3 zeta | RVKFSRSADAPAYQQGQNQL | 69 |
| variant | YNELNLGRREEYDVLDKRRG | |
| (delta23) | RDPEMGGK | |
| CD3 zeta | RVKFSRSADGVYNALQKDKM | 70 |
| variant | AEAYSEIRGRDPEMGGK | |
| (delta13) | | |
| R, rituximab | CPYSNPSLC | 71 |
| mimotope | | |
| GGGGS)3 | GGGGSGGGGSGGGGS | 72 |
| GGGGS linker | GGGGS | 73 |
| (GGGGS)4 | GGGGSGGGGGGGGSGGGGS | 74 |
| Witlow linker | GSTSGSGKPGSGEGSTKG | 75 |
| Q, QBEND-10 | ELPTQGTFSNVSTNVS | 76 |
| epitope | | |
| Q, QBEND-10 | ELPTQGTFSNVSTNVSPAKP | 77 |
| epitope | TTTA | |
| QR3 | GSGGGGSCPYSNPSLCSGGG | 78 |
| | GSELPTQGTFSNVSTNVSPA | |
| | KPTTTACPYSNPSLC | |
| QQ | GSGGGGSELPTQGTFSNVST | 79 |
| | NVSPAKPTTTAGSGGGGSEL | |
| | PTQGTFSNVSTNVSPAKPTT | |
| | TA | |
| CD28 hinge | IEVMYPPPYLDNEKSNGTII | 80 |
| | HVKGKHLCPSPLFPGPSKP | |
| CD28 TM | FWVLVVVGGVLACYSLLVTV | 81 |
| | AFIIFWV | |
| 4F11 scFv | QVTLKESGPVLVKPTETLTL | 82 |
| | TCTVSGFSLSNARMGVTWIR | |
| | QPPGKALEWLAHIFSNDEKS | |
| | YSTSLKSRLTISKDTSKTQV | |
| | VLTMTNMDPVDTATYYCARI | |
| | RDYYDISSYYDYWGQGTLVS | |
| | VSSGGGGSGGGGSGGGGSGG | |
| | GGSDIQMTQSPSAMSASVGD | |
| | RVTITCRASQDISNYLAWFQ | |
| | QKPGKVPKRLIYAASSLQSG | |
| | VPSRFSGSGSGTEFTLTISS | |
| | LLPEDFATYYCLQLNSFPFT | |
| | FGGGTKVEIN | |
| 8F8 scFv | QVQLQESGPGLVQPSETLSL | 83 |
| | TCTVSGGSISYYYWSWIRQP | |
| | PGKGLEWIGNINYMGNTIYN | |
| | PSLKSRVTISVDTSKDQFSL | |
| | KLTSVSAADTAVYYCVRAEG | |
| | SIDAFDFWGQGTLVAVSLGG | |
| | GGSGGGGSGGGGSGGGGSDI | |
| | QMTQSPSTLSASVGDRVTIT | |
| | CRASQSISSWLAWYQQKPGK | |
| | APKVLIYKASNLESGVPSRF | |
| | SGSGSGTEFTLTISSLQPDD | |
| | FATYYCQQYNSYSCTFGQGT | |
| | KLEIK | |
| CD70 | MPEEGSGCSVRRRPYGCVLR | 84 |
| | AALVPLVAGLVICLVVCIQR | |
| | FAQAQQQLPLESLGWDVAEL | |
| | QLNHTGPQQDPRLYWQGGPA | |
| | LGRSFLHGPELDKGQLRIHR | |
| | DGIYMVHIQVTLAICSSTTA | |
| | SRHHPTTLAVGICSPASRSI | |
| | SLLRLSFHQGCTIASQRLTP | |
| | LARGDTLCTNLTGTLLPSRN | |
| | TDETFFGVQWVRP | |
| 4F11 scFv | QVTLKESGPVLVKPTETLTL | 85 |
| | TCTVSGFSLSNARMGVTWIR | |
| | QPPGKALEWLAHIFSNDEKS | |
| | YSTSLKSRLTISKDTSKTQV | |
| | VLTMTNMDPVDTATYYCARI | |
| | RDYYDISSYYDYWGQGTLVS | |
| | VSSGGGGSGGGGSGGGGSDI | |
| | QMTQSPSAMSASVGDRVTIT | |
| | CRASQDISNYLAWFQQKPGK | |
| | VPKRLIYAASSLQSGVPSRF | |
| | SGSGSGTEFTLTISSLLPED | |
| | FATYYCLQLNSFPFTFGGGT | |
| | KVEIN | |
| 4F11z | MALPVTALLLPLALLLHAAR | 86 |
| | PQVTLKESGPVLVKPTETLT | |
| | LTCTVSGFSLSNARMGVTWI | |
| | RQPPGKALEWLAHIFSNDEK | |
| | SYSTSLKSRLTISKDTSKTQ | |
| | VVLTMTNMDPVDTATYYCAR | |
| | IRDYYDISSYYDYWGQGTLV | |
| | SVSSGGGGSGGGGSGGGGSD | |
| | IQMTQSPSAMSASVGDRVTI | |
| | TCRASQDISNYLAWFQQKPG | |
| | KVPKRLIYAASSLQSGVPSR | |
| | FSGSGSGTEFTLTISSLLPE | |
| | DFATYYCLQLNSFPFTFGGG | |
| | TKVEINTTTPAPRPPTPAPT | |
| | IASQPLSLRPEACRPAAGGA | |
| | VHTRGLDFACDIYIWAPLAG | |
| | TCGVLLLSLVITLYCRVKFS | |
| | RSADAPAYQQGQNQLYNELN | |
| | LGRREEYDVLDKRRGRDPEM | |
| | GGKPRRKNPQEGLYNELQKD | |
| | KMAEAYSEIGMKGERRRGKG | |
| | HDGLYQGLSTATKDTYDALH | |
| | MQALPPR | |
| 4F11 Variable | DIQMTQSPSAMSASVGDRVT | 87 |
| light chain | ITCRASQDISNYLAWFQQKP | |
| | GKVPKRLIYAASSLQSGVPS | |
| | RFSGSGSGTEFTLTISSLLP | |
| | EDFATYYCLQLNSFPFTFGG | |
| | GTKVEIN | |
| 4F11 Variable | QVTLKESGPVLVKPTETLTL | 88 |
| heavy | TCTVSGFSLSNARMGVTWIR | |
| chain | QPPGKALEWLAHIFSNDEKS | |
| | YSTSLKSRLTISKDTSKTQV | |
| | VLTMTNMDPVDTATYYCARI | |
| | RDYYDISSYYDYWGQGTLVS | |
| | VSS | |
| 41BB intracellular | GRKKLLYIFKQPFMRPVQTT | 89 |
| signaling | QEEDGCSCRFPEEEEGGCEL | |
| domain | | |
| (ISD) | | |
| CD3ζ intracellular | RVKFSRSADAPAYKQGQNQL | 90 |
| signaling | YNELNLGRREEYDVLDKRRG | |
| domain | RDPEMGGKPRRKNPQEGLYN | |
| (ISD) | ELQKDKMAEAYSEIGMKGER | |
| | RRGKGHDGLYQGLSTATKDT | |
| | YDALHMQALPPR | |

### EXAMPLES

### Example 1. Protective Effect of a CD58 Knockout (KO)+RFX5 or NLRC5 KO in Non-CAR T Cells

The protective effect of CD58 KO to enhance survival as a single modification (single KO) or to provide further survival benefit of an RFX5 KO or an NLRC5 KO against host rejection was tested using primed T and natural killer (NK) mixed lymphocyte reaction (MLR) assays.

PBMCs from HLA-A2-expressing (HLA-A2+) healthy human donors were used as allogeneic effectors (host), and PBMCs from non-HLA-A2-expressing (HLA-A2−) healthy donors were used to generate target cells (graft). Gene editing was performed to generate graft T cells with the following KOs: TRAC KO, TRAC/CD58 KO, TRAC/β2m KO, TRAC/RFX5 KO, TRAC/NLRC5 KO, TRAC/β2m/CD58 KO, TRAC/RFX5/CD58 KO, and TRAC/NLRC5/CD58 KO via CRISPR/Cas9. After 1 week in vitro expansion, target cells were then purified to remove any TCR α/β expressing cells. The level of specific gene knockout or MHC-I and MHC-II knockdown was evaluated via FACS.

Alloreactive T cells are thought to be the main mediators of allorejection. Therefore, primed alloreactive T cell mixed lymphocyte reactions (MLRs) were performed using the gene-edited cells as target cells to evaluate the protective effect of the different knockouts on survival against the primed alloreactive T cells. To generate primed effector T cells, allogeneic PBMCs from HLA-A2+ donors were co-cultured with irradiated PBMCs or pan T cells isolated from the graft donors for 1 week to promote outgrowth and expansion of alloreactive T cells. Effector pan T cells were then purified out and co-incubated with gene-edited graft T cells at 1:1 ratio in R10+20 IU/mL IL-2 for 2 days. The extent of killing was determined by analyzing the absolute number of surviving graft T cells by gating on live HLA-A2-TCRαβ− CD4+CD8+ and desired gene edits (e.g., CD58 KO) (FIG. 1E).

As shown in FIG. 1E, a CD58 KO enhanced survival of RFX5 KO cells or NLRC5 KO cells as compared to a single RFX5 KO or a single NLRC5 KO.

In an NK MLR assay, human NK cells were isolated from freshly isolated HLA-A2+ human PBMCs via MACS purification (Miltenyi, human pan NK cell isolation kit, Cat #130-092-657). In a 96-well plate, 20,000 graft T cells were seeded with 20,000 or 100,000 host NK cells and cultured in R10+1000 IU/mL IL-2 for 2 days. Survival of graft T cells was determined by flow cytometry using absolute counts by gating on live HLA-A2-TCRαβ− CD56− CD4+CD8+ and desired gene edits (e.g., CD58 KO).

As shown in FIG. 1F, a CD58 KO enhanced survival of RFX5 KO cells or NLRC5 KO cells when compared to a single RFX5 KO cells or a single NLRC5 KO cells. A single CD58 KO did not affect graft cell survival in NK MLR. Similar trends were observed for both an effector cell:target cell ratio (E:T) of 1:1 and E:T=5:1.

### Example 2. Protective Effect of CD48 KO or ICAM-1 KO+RFX5 KO in Non-CAR T Cell Grafts

Pan T cells from HLA-A2− donors were used for target cell generation. Gene editing was performed to generate target cells with the following KOs: TRAC KO, TRAC/RFX5 KO, TRAC/CD48 KO, TRAC/ICAM-1 KO, TRAC/RFX5/CD48 KO, and TRAC/RFX5/ICAM-1 KO via CRISPR/Cas9. After in vitro expansion for 2 weeks, targets were then purified to remove any TCR α/β expressing cells. The level of specific gene knockout or MHC-I and MHC-II knockdown was evaluated via FACS.

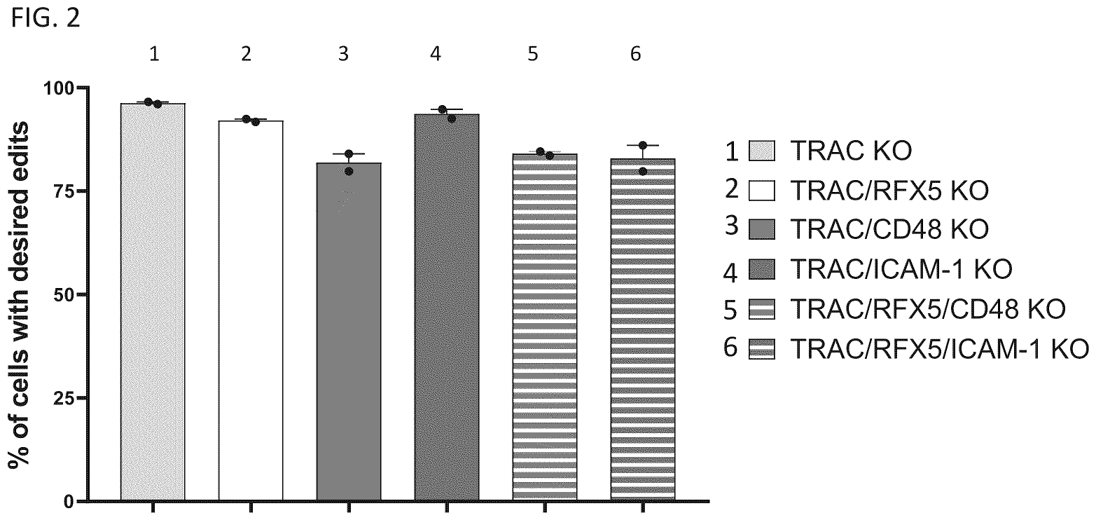

As shown in FIG. 2, high gene editing efficiency was observed for all groups (based on an average of two donors). More than 80% of cells at day 13 of production had the desired gene edits. While an RFX5 KO was used in this set of testing, no NLRC5 KO and no β2m KO were used.

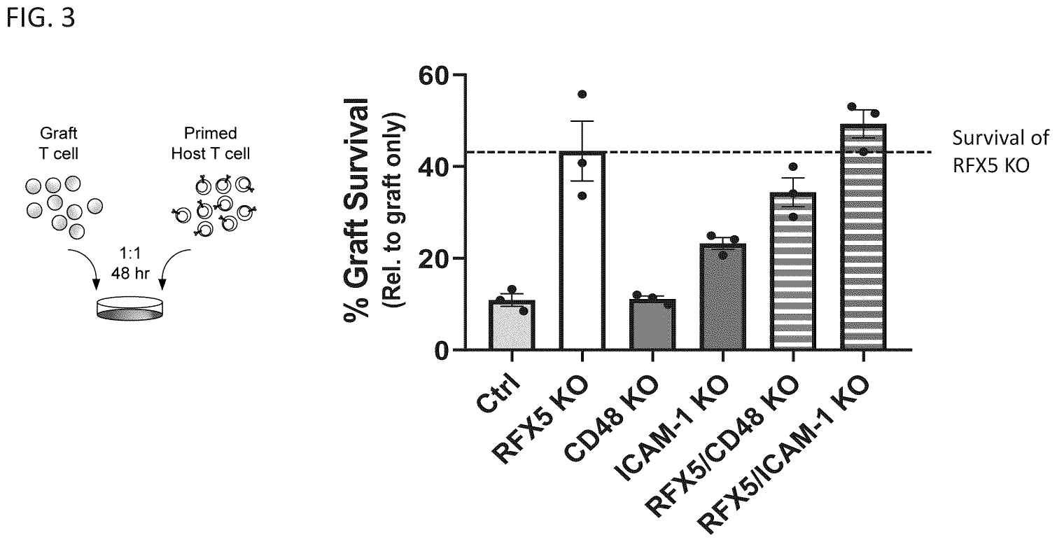

The protective effect of ICAM-1 or CD48 KO on enhancing survival of an RFX5 KO against allogeneic T cell rejection was tested using a primed T cell MLR assay as described in Example 1. As shown in FIG. 3, an ICAM-1 KO may slightly enhance survival of RFX5 KO as shown in a primed T cell MLR assay as compared to a CD48 KO. As single KOs, ICAM-1 KO enhanced target cell survival compared to control graft (TRAC KO). Survival of CD48 KO was similar to control graft.

The protective effect of ICAM-1 KO to further enhance the survival of RFX5 KO against host PBMC rejection as compared to a CD48 KO was tested in a PBMC MLR assay. Survival of gene-edited graft cells in the presence of allogeneic PBMC donors (n=3) was assessed. The effector:target (E:T) ratio used was 10:1. Survival of graft cells was quantified by FACS 9 days after the co-culture. As shown in FIG. 4, an ICAM-1 KO further enhanced survival of RFX5 KO as shown in a PBMC MLR assay as compared to a CD48 KO.

### Example 3. Protective Effect of CD58 KO or ICAM-1 KO+RFX5 KO Using CAR T Cell Grafts

The protective effect of CD58 KO to enhance CAR T cell survival as a single modification (single KO) or to provide further survival benefit of an RFX5 KO or an NLRC5 KO against T cell rejection was tested using a primed T MLR assay. CD19 CAR T cells (CD19 FMC63) expressing a turbodomain, which is a constitutively active chimeric cytokine receptor (CACCR) that includes a TpoR domain variant (478-582; H499L; S505N; W515K) and an intracellular IL2Rb domain (339-379,393-433,518-551) (see Lin et al., US US 2021-0260118 A1, which is incorporated herein by reference in its entirety), were engineered from healthy donor cells. These CAR T cells will be hereinafter referred to as CD19 CAR T cells in this Example. The expression of the CAR and turbodomain was under the control of either an EF1alpha or a PGK promoter as noted below.

The CD19 CAR T cells were modified to introduce one or more knockouts and then used as target cells. Specifically, the CD19 CAR T cells were gene-edited to provide the following knockouts: TRAC KO, TRAC/β2m KO, TRAC/CD58 KO, TRAC/RFX5 KO, TRAC/NLRC5 KO, TRAC/β2m/CD58 KO, TRAC/CD58/RFX5 KO, and TRAC/CD58/NLRC5 KO via CRISPR/Cas9.

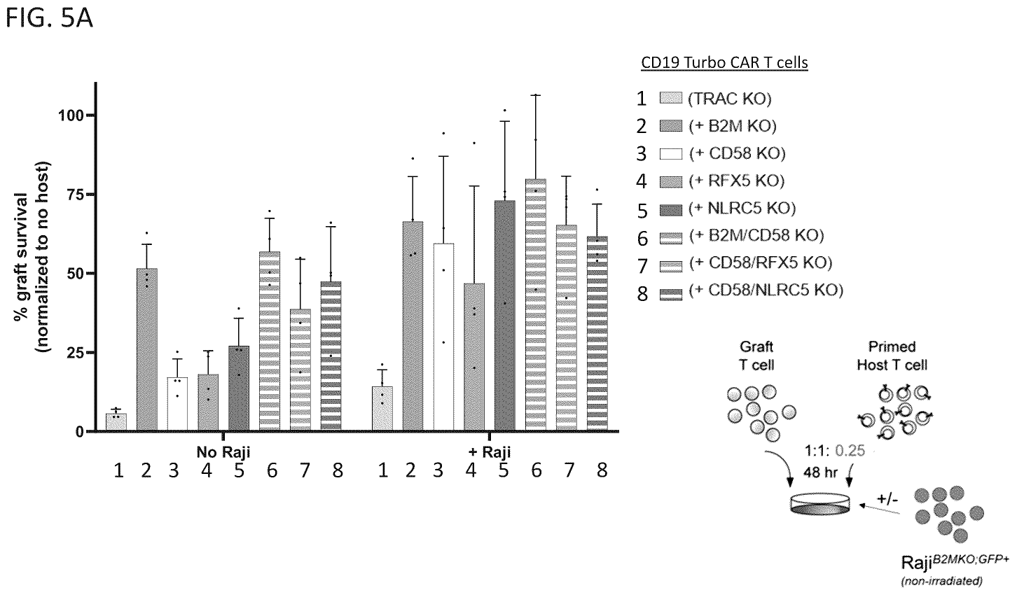

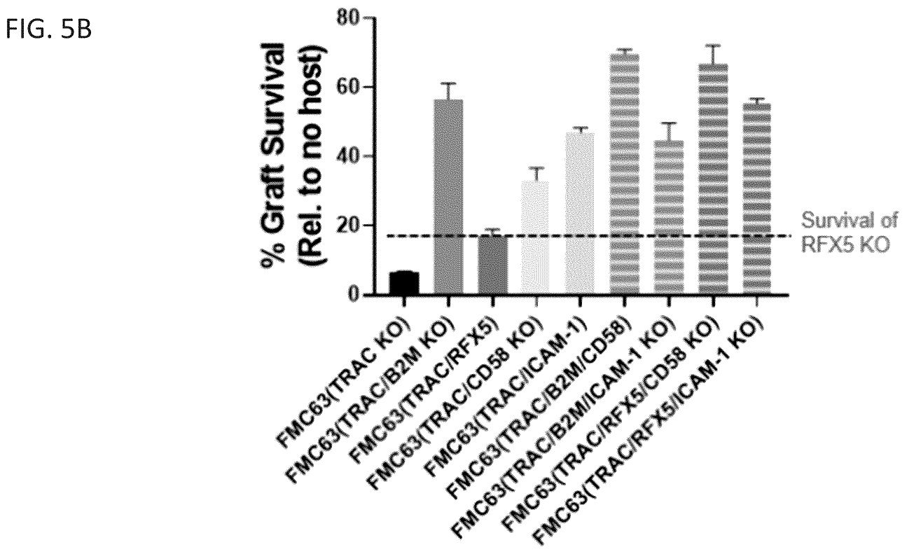

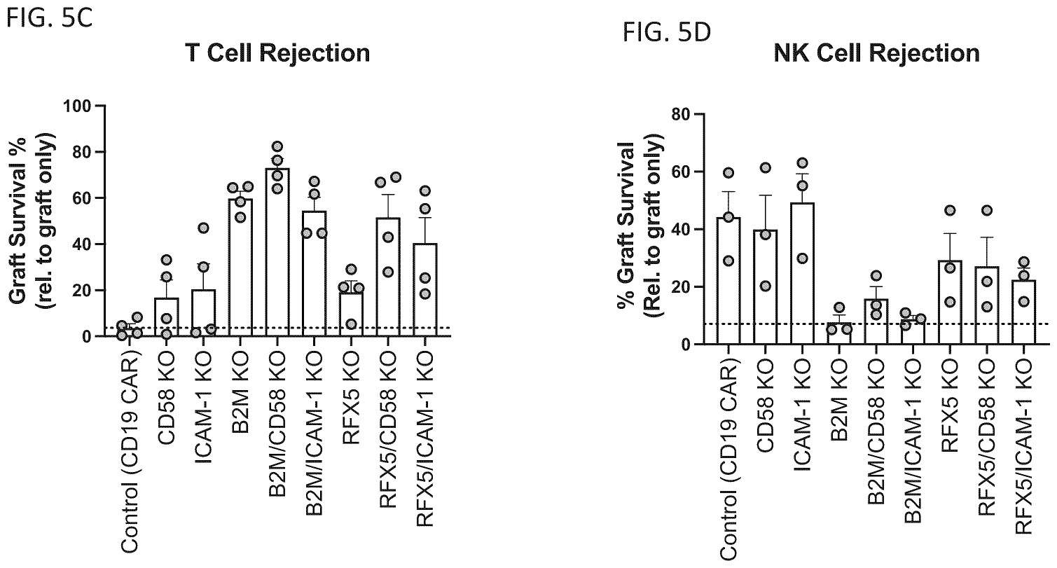

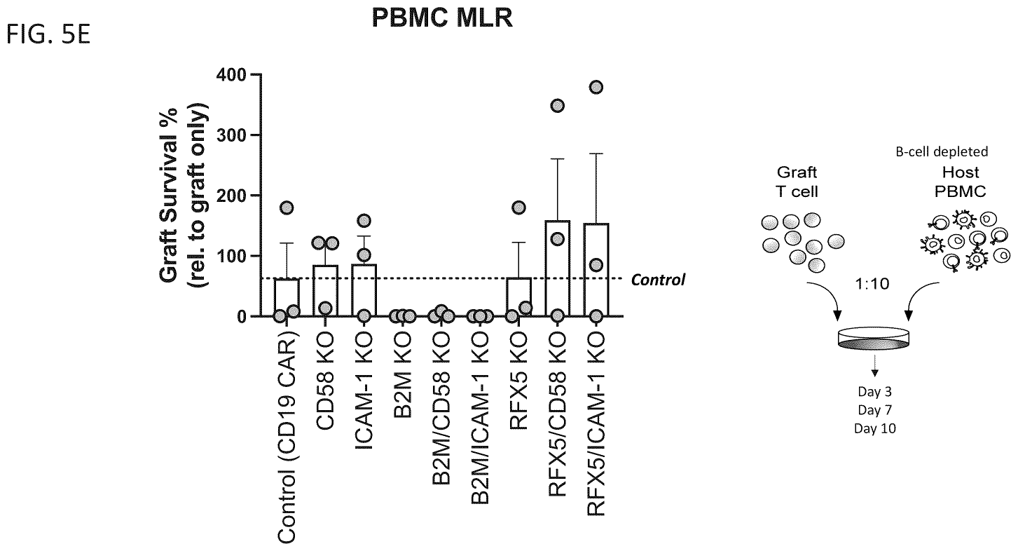

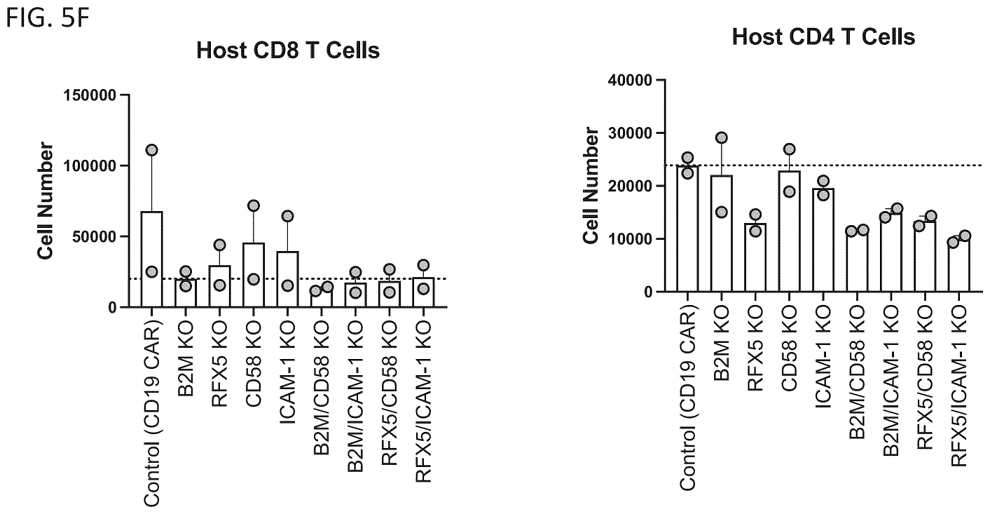

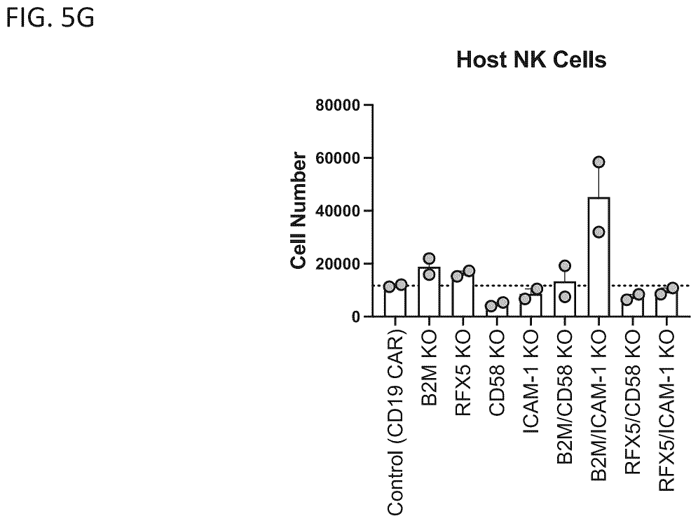

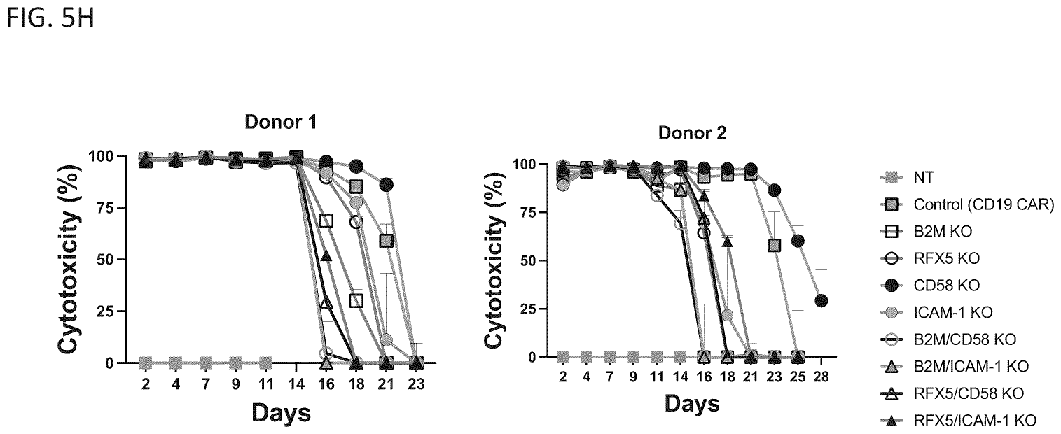

For the experimental data presented in FIGS. 5A-J, the CD19 CAR T cells were engineered to have an EF1alpha promoter for controlling expression of the CAR and turbodomain.

A primed T MLR assay was performed to test the effectiveness of gene-edited CD19 CAR T cells (as target cells) against allogeneic T cell rejection. Data was pooled from 4 graft/host donor pairs: one graft and 4 hosts. Cells were cultured at effector:target (E:T)=1:1 ratio in the presence of 20 IU/mL of IL-2 for 2 days. In some cases, the CAR T cells were activated using GFP-expressing β2m KO Raji tumor cells. Survival of graft CAR T cells were assessed by FACS and gated as live GFP-HLA-A2-TCRαβ− CD4+CD8+CAR+ and desired gene edits (e.g., CD58 KO).

As shown in FIG. 5A, a single β2m KO, a single CD58 KO, a single NLRC5 KO, and a single RFX5 KO all enhance graft cell survival compared to control CAR T graft (TRAC KO). In addition, when the CD19 CAR T cells were not activated (without Raji tumor cells), a CD58 KO further enhances survival versus a single NLRC5 KO or a single RFX5 KO CAR T cells. Activated CD19 CAR T cells (in the presence of Raji tumor cells) survive better than non-activated CAR T cells (no Raji tumor cells). All cloaking KO graft cells, e.g., a CD58 KO, survived relatively comparable, and all survived significantly better than control graft (TRAC KO).

The protective effect of CD58 KO or ICAM-1 KO to enhance CAR T cell survival as a single modification (single KO) or to provide further survival benefit of an RFX5 KO against T cell rejection was tested using a primed T MLR assay.

The CD19 CAR T cells were gene-edited to provide the following knockouts: TRAC KO, TRAC/β2m KO, TRAC/RFX5 KO, TRAC/CD58 KO, TRAC/ICAM-1 KO, TRAC/β2m/CD58 KO, TRAC/β2m/ICAM-1 KO, TRAC/RFX5/CD58 KO, and TRAC/RFX5/ICAM-1 KO.

A primed T MLR assay was performed to test the effectiveness of gene-edited CD19 CAR T cells against allogeneic T cell rejection. Data shown was average of technical triplicates of one representative graft/host pair from 3 unique pairs. Cells were cultured at effector:target=1:1 ratio in the presence of 20 IU/mL of IL-2 for 2 days. The CAR T cells were activated using GFP-expressing β2m KO Raji tumor cells. Survival of graft CAR T cells were assessed by FACS and gated as live GFP-HLA-A2-TCRαβ− CD4+CD8+CAR+ and desired gene edits (e.g., CD58 KO).

As shown in FIG. 5B, a single β2m KO, a single RFX5 KO, a single CD58 KO, and a single ICAM-1 KO all enhance graft CAR T cells survival compared to control (TRAC KO). CD58 KO provided slight added benefit to the survival of β2m KO, while ICAM-1 KO did not. Both CD58 KO and ICAM-1 KO provided substantial added survival benefit to RFX5 KO CAR T cells, compared to RFX5 single KO graft.

The CD19 CAR T cells (expressing a turbodomain as described above) were engineered to introduce different knockout combinations of B2M, RFX5, CD58, and NLRC5 and then used as graft cells in a primed T MLR assay, an NK cell MLR assay and a PBMC MLR assay. Specifically, the CD19 CART cells were gene-edited to provide the following knockouts: TRAC KO, TRAC/CD58 KO, TRAC/ICAM-1 KO, TRAC/β2m KO, TRAC/β2m/CD58 KO, TRAC/β2m/ICAM-1 KO, TRAC/RFX5 KO, TRAC/RFX5/CD58 KO, and TRAC/RFX5/ICAM-1 KO via CRISPR/Cas9.

For the primed T MLR assay, a ratio of 1:1:0.25 for host:graft:Raji tumor cells (WT Raji cells having a B2M knockout) was used. Primed host T-cells were enriched 2× using Miltenyi Pan T Isolation kit. A readout was performed after 2 days. As shown in FIG. 5C, CD58 KO and ICAM-1 KO resulted in improved survival of CAR T cells in the T cell MLR assay as single modifications or in combination with a RFX5 knockout (each dot represents one unique allogeneic host/graft pair). The combination of RFX5, CD58, and ICAM-1 were found to significantly mitigate T cell rejection compared to an RFX5 knockout alone. In addition, a CD58/RFX5 double knockout was found to specifically mitigate T cell rejection to almost the same extent as B2M knockout (KO) alone cells.

For the primed NK MLR assay, a ratio of 10:1:0.25 for NK cells:graft:Raji tumor cells (WT Raji cells) was used. Frozen NK cells were thawed for use. A readout was performed after 2 days (1,000 IU/ml IL-2). As shown in FIG. 5D, B2M knockout cells were found to be sensitive to NK cell killing, as expected (each dot represents one graft/NK pair). CD58 and ICAM-1 knockout cells, either alone or in combination, with other knockout targets, i.e., B2M and RFX5, did not exhibit any additional NK cell rejection, as compared to the B2M knockout. These results suggest that the RFX/CD58 or ICAM-1 double knockouts could potentially mitigate T cell rejection to the same extent as B2M KO but without eliciting the same magnitude of NK cell rejection, potentially mitigating rejection by both T and NK cells.

These assays analyzed graft survival after coculture with primed T cells or primed NK cells. The CD58 and ICAM 1 knockouts were found to decrease T cell-mediated rejection both as single knockouts and in an RFX5 KO background. In the NK MLR, the CD58 and ICAM-1 knockouts were not found to have a detrimental effect on graft survival (FIGS. 5C-D).

For the PBMC MLR assay, a ratio of 10:1 for host:graft cells was used for a 10-day co-culture. PBMCs from 3 different donors were depleted of B cells prior to co-culture. A readout was performed after 10 days. As shown in FIG. 5E, a CD58 knockout and an ICAM-1 knockout improve graft survival as single modifications or in combination with an RFX5 knockout (each dot represents one unique allogeneic host/graft pair—graft, 3 host donors). As shown in FIG. 5F, the addition of a CD58 knockout with either a B2M knockout or an RFX5 knockout was found to decrease host immune cell expansion in 2 of the 3 donors (left panel—host CD8+ cells; right panel—host CD4+ cells). As shown in FIG. 5G, host NK cells expand to a greater degree when co-cultured with graft cells that have a B2M knockout alone or in combination with other knockouts. The addition of a CD58 knockout alone or in combination with an RFX5 knockout was found to reduce expansion.

In another PBMC MLR assay using the CD19 CAR T cells, the effect of different knockouts on different subsets of immune cells was evaluated. PBMCs from 3 different donors were depleted of B cells prior to co-culture. A ratio of 10:1 host:graft cells was used for a 10-day culture. A readout was performed after 10 days. The TRAC/CD58 KO and TRAC/ICAM-1 KO results were measured on a separate plate (error bars were removed for ease of visualization).

As shown in FIG. 5H, host CD8+ cells expanded to a lesser degree when co-cultured with graft cells that have different knockouts and knockout combinations. For instance, the addition of a combined RFX5/CD58 knockout reduced expansion as compared to the control (FMC63; TRAC KO graft). As shown in FIG. 5I, host CD4+ cells expanded to a lesser degree when co-cultured with graft cells that have different knockouts and knockout combinations. For instance, the addition of a combined RFX5/CD58 knockout reduced expansion as compared to the control (FMC63; TRAC KO graft).

In vitro long-term killing assays were performed using the CD19 CAR T cells against target cells, e.g., Raji WT cells or Raji CD19^{low} cells, at a E:T ratio of 8:1. As shown in FIG. 5J, the CD19 CAR T cells containing different knockouts demonstrated cytotoxic activity against the Raji WT target cells. The control CAR T cells with a TRAC knockout alone demonstrated effective killing compared to most of the other CAR T cells having additional knockouts. Surprisingly, the CAR T cells with a TRAC/CD58 knockout conferred similar or slightly better killing than the control CAR T cells. Similar results were obtained when Raji CD19^{low} cells were used as target cells.

### Example 4. Protective Effect of CD58 KO or ICAM-1 KO+RFX5 KO Against NK Cell Rejection

The CD19 CAR T cells described in Example 3 (expressing a turbodomain) were modified to introduce one or more knockouts and then used as target cells. The expression of the CAR and turbodomain was under the control of either an EF1alpha or a PGK promoter as noted below.

Specifically, the CD19 CAR T cells were gene-edited to provide the following knockouts: TRAC KO, TRAC/β2m KO, TRAC/RFX5 KO, TRAC/CD58 KO, TRAC/ICAM-1 KO, β2m/CD58 KO, β2m/ICAM-1 KO, RFX5/CD58 KO, and RFX5/ICAM-1 KO via CRISPR/Cas9.

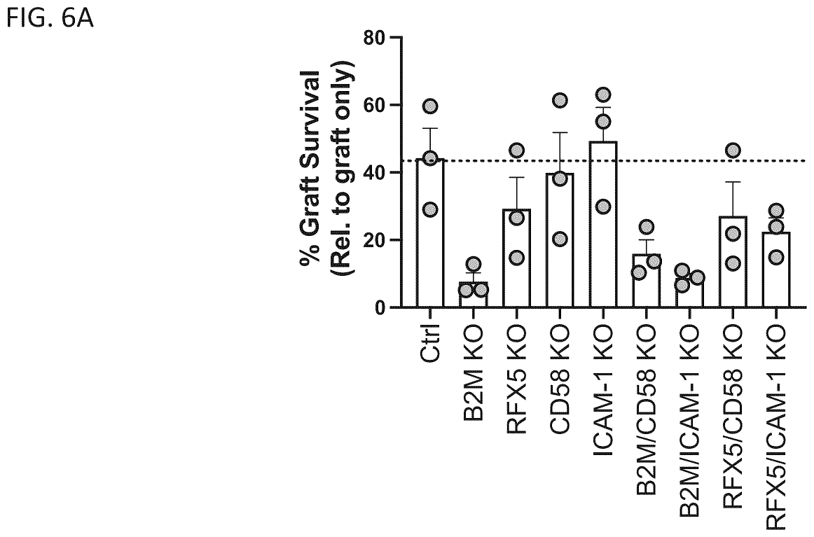

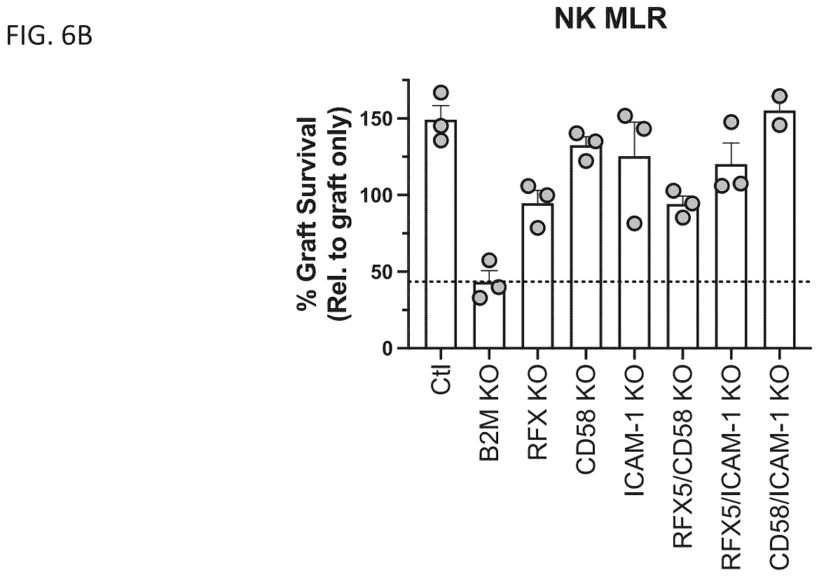

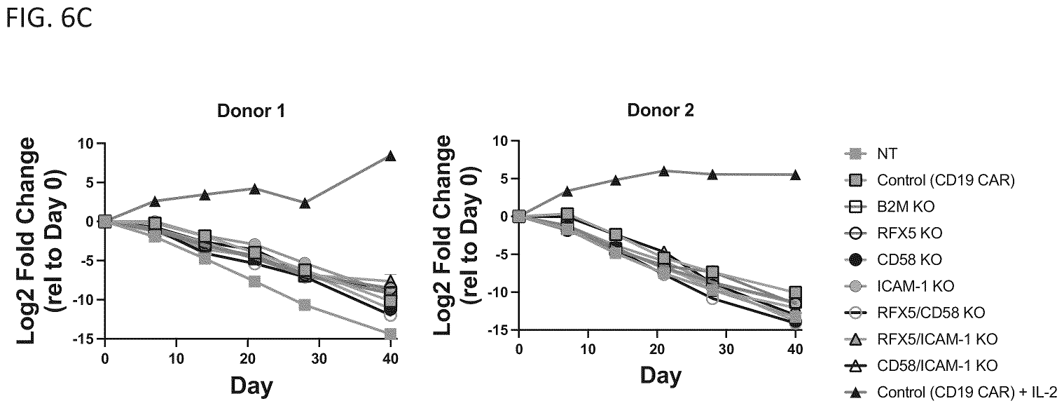

For the experiments presented in FIG. 6A, an EF1alpha promoter was used to control expression of the CAR and turbodomain. For the experiments presented in FIG. 6B, a PGK promoter was used to control expression of the CAR and turbodomain.

An NK MLR assay was used to test survival of the gene-edited CD19 CAR T cell graft against allogeneic NK rejection. Data was pooled from 3 graft/host donor pairs: one graft and 3 hosts. NK cells isolated from frozen PBMCs were co-cultured with the gene-edited CD19 CAR T cells in the presence of 1,000 IU/mL of IL-2 for 2 days. The CD19 CAR T cells were activated using GFP-expressing Raji tumor cells. The ratio of NK cells:graft:Raji cells was 10:1:0.25. Survival of graft CAR T cells were assessed by FACS and gated as live GFP-HLA-A2-TCRαβ-CD56− CD4+CD8+CAR+ and desired gene edits (e.g., CD58 KO).

As shown in FIG. 6A, a single β2m KO incurred substantial NK rejection which resulted in low survival, while a RFX5 single KO incurred some NK rejection but to a less extent. A single CD58 KO or ICAM-1 KO had similar survival compared to control graft (TRAC KO). CD58 KO slightly rescued β2m KO from NK rejection. CD58 KO or ICAM-1 did not show substantial rescue of RFX5 KO survival against NK rejection.

In another NK cell MLR, fresh NK cells were used. A ratio of 1:1:0.25 was used for NK cells: graft: Raji target cells. NK cells were co-cultured with gene-edited CD19 CAR T cells in the presence of 1,000 IU/ml IL-2 for 2 days. As shown in FIG. 6B, a CD58 knockout and an ICAM-1 knockout do not incur an NK cell rejection (each dot represents one graft/NK pair). In addition, the CAR T knockout cells were not found to exhibit IL-2 independent growth (FIG. 6C).

TABLE 4.1
List of sgRNA sequences

| SEQ ID | Gene used | sgRNA sequence |
|---|---|---|
| NO: | for | (5′-3′) |
| 91 | TRAC | gcugguacacggcaggguca |
| 92 | B2M (β2m) | ucacgucauccagcagagaa |
| 93 | NLRC5 | cagcucugcaaggcucuggg |
| 94 | RFX5 | aguacauguaugccuauagg |
| 95 | CD58 | aaggcacauugcuugguaca |
| 96 | CD48 | ucacuugguacauaugaccg |
| 97 | ICAM-1 | ugagggggacacagaugucu |

### Example 4.1—Cytotoxic Properties of CAR T Cell Knockouts

The CD19 CAR T cells described in Example 3 (expressing a turbodomain) were modified to introduce one or more knockouts. The CD19 CAR T cells were engineered from pan T cells from two HLA-A2− donors. The expression of the CAR and turbodomain was under the control of either an EF1alpha or a PGK promoter as noted below.

Specifically, the CD19 CAR T cells were gene-edited to provide the following knockouts: TRAC KO, TRAC/β2m KO, TRAC/RFX5 KO, TRAC/CD58 KO, TRAC/ICAM-1 KO, TRAC/RFX5/CD58 KO, TRAC/RFX5/ICAM-1 KO, and TRAC/CD58/ICAM-1 KO via CRISPR/Cas9.

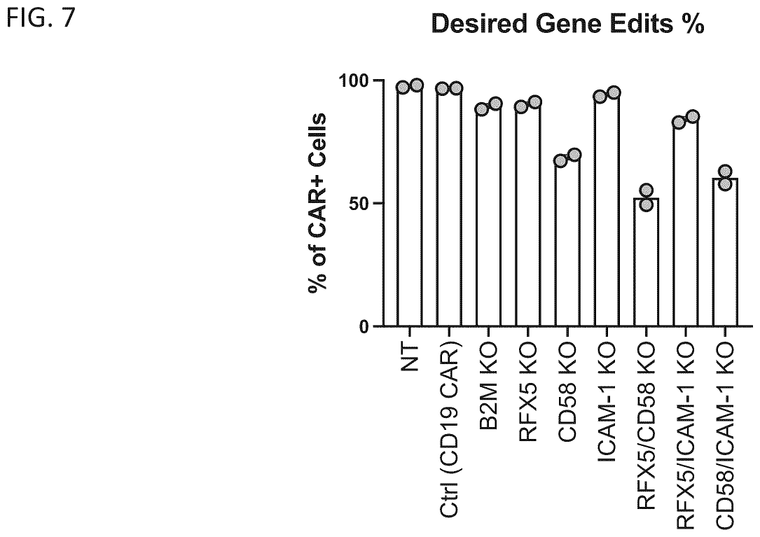

For the experimental data presented in FIG. 7-12, the CD19 CAR T cells were engineered to have a PGK promoter for controlling expression of the CAR and turbodomain.

The TRAC knockout was common to all edited cells. After in vitro expansion for 16 days, gene-edited cells were then purified to remove any TCR α/β expressing cells. The level of specific gene knockout or MHC-I and MHC-II knockdown was evaluated via flow cytometry.

As shown in FIG. 7, high gene editing efficiency was observed for all groups (based on an average of two donors). More than 50% of cells at day 16 of production had the desired gene edits. For all knockout conditions listed above, 1×10⁶ cells were edited and then expanded. As shown in FIG. 8, expansion of the edited cells was not affected by any of the various knockouts as compared to the TRAC knockout control.

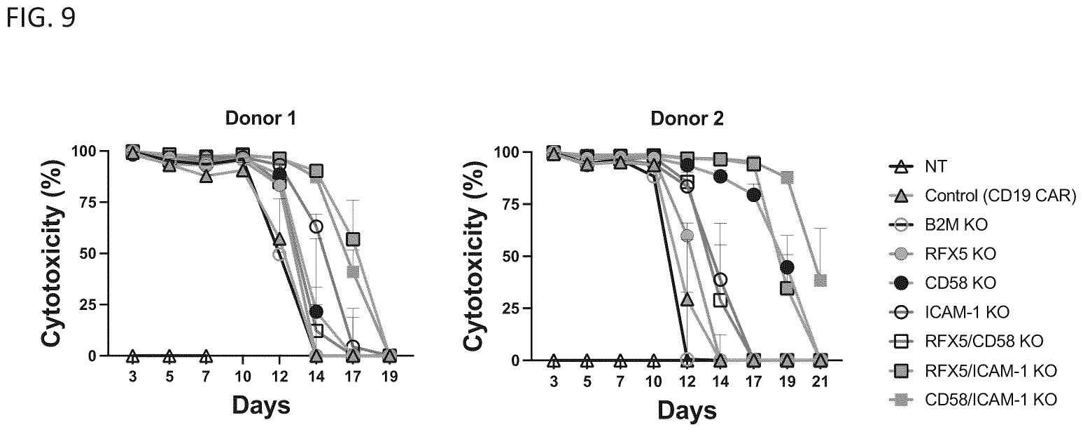

In vitro long-term killing assays were performed using the CD19 CAR T cells with various knockouts against target cells, e.g., Raji WT or Raji CD19^{low} cells, at a E:T ratio of 8:1. As shown in FIG. 9, the various CAR T cells containing different knockouts demonstrated cytotoxic activity against the Raji WT target cells. All of the CAR T knockout cells were found to demonstrate equal or superior cytotoxicity as compared to the control CAR T cells (TRAC knockout only). CAR T cells were co-cultured with luciferase-GFP-expressing Raji cells at an effector-to-target ratio of 8:1. Every 2-3 days, half the cells were passaged onto fresh Raji cells. The remaining half of cells was then used to determine target cell killing using Bright-glo reagent (Promega).

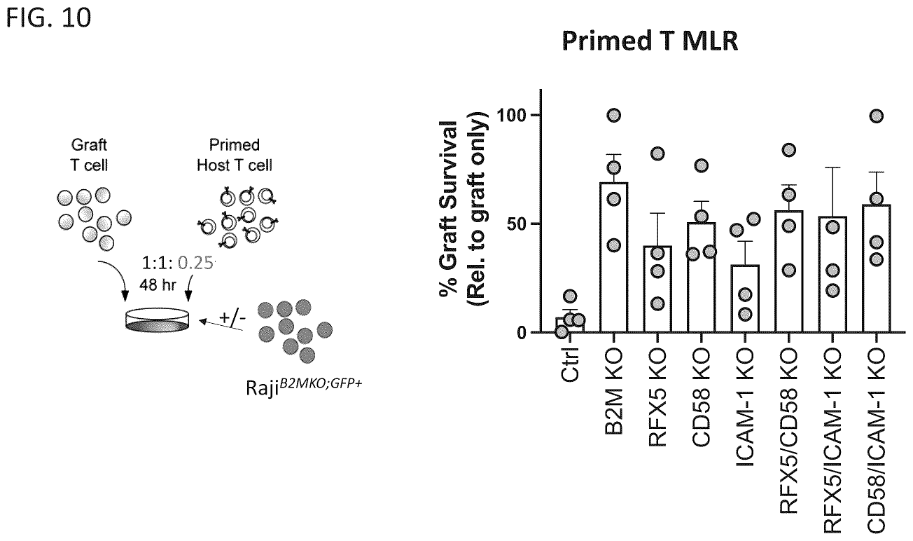

A series of primed T MLR assays were performed with several gene-edited CD19 CAR T cells having various knockouts. Primed host T cells were isolated 2× with Miltenyi Pan T isolation kit 7 days after co-culture with WT Raji cells having a B2M knockout irradiated graft pan T cells. A ratio of 1:1:0.25 for T cell host:graft:Raji (B2M knockout) was used and a readout was obtained on day 2. As shown in FIG. 10, a CD58 knockout was found to effectively mitigate T cell rejection as a single knockout or in combination with other knockouts (each dot represents one graft/host pair).

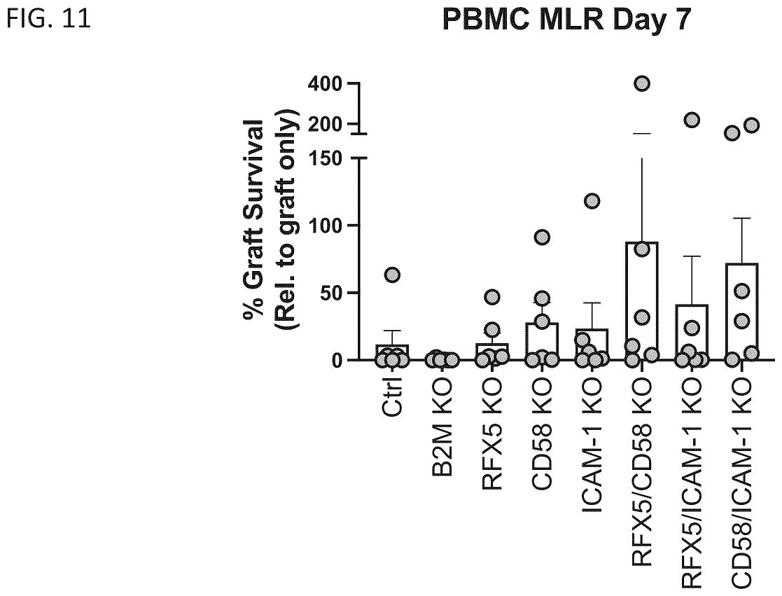

PBMC MLR assays were also performed. A ratio of 10:1 host:graft cells was used for a 10-day culture. PBMCs from a donor were depleted of B cells prior to co-culture. Readouts were performed on days 3, 7 and 9. As shown in FIG. 11, the graft CAR T cells with various knockouts demonstrated a survival advantage over the control and the B2M knockout at day 7. The combined knockouts (e.g., RFX5/CD58, RFX5/ICAM-1, and CD58/ICAM-1) demonstrated better survival against rejection than the single knockouts (e.g., RFX5, CD58, and ICAM-1). The CD58 knockout cells demonstrated the best survival among the single knockout graft cells. The CD58 knockout in combination with an RFX5 knockout or an ICAM-1 knockout demonstrated the best survival against rejection at day 9 (data not shown). In addition, the single CD58 knockout showed the best survival at day 9 as compared to other single knockouts (data not shown).

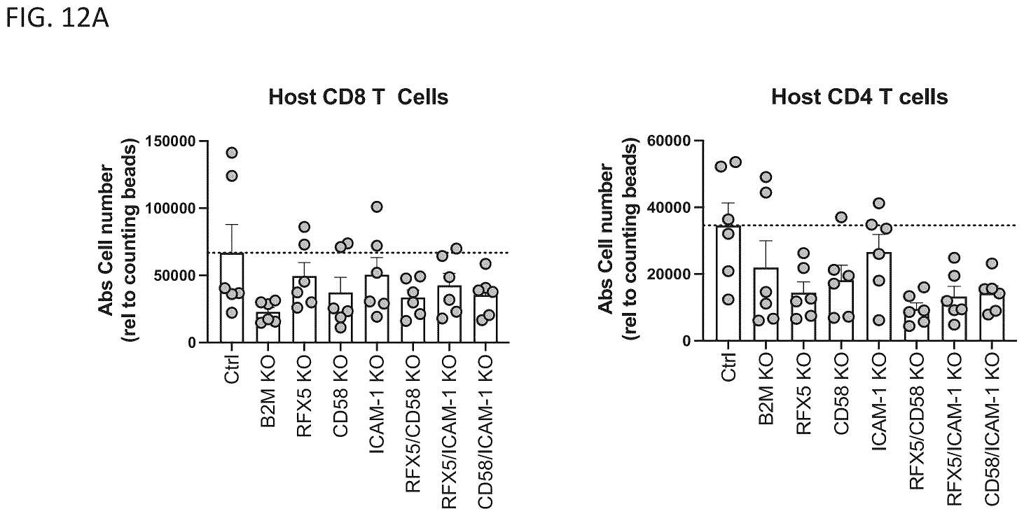

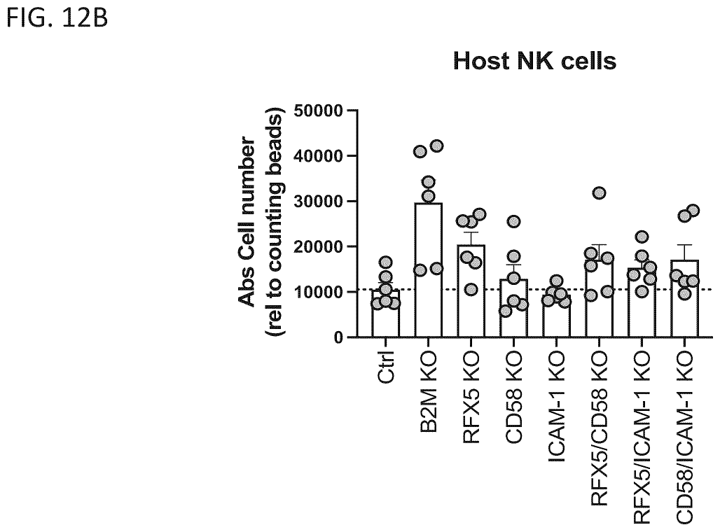

The ability of the different gene-edited CAR T cells to impact the expansion of host immune cells was evaluated. As shown in FIG. 12A-12B, the CAR T cells were found to reduce the expansion of host CD8+ and host CD4+ cells (A). The B2M knockout CAR T cells were found to be the least effective at reducing host NK cell expansion when compared to other knockout cells (B).

In addition, CD19 CAR T cells were engineered to have an EF1alpha promoter for controlling expression of the CAR and turbodomain. These cells were also tested for their cytotoxic ability and found to demonstrate similar cytotoxic ability (data not shown).”

### Example 4.2—CD19 CAR/CD70 Binding Protein T Cells with a CD58 Knockout

Transduction of LVV constructs into PBMCs results in engineered cells with randomly integrated transgene(s) in the host cell genome. The transgene(s) can be introduced into cells by site-specific integration (SSI) into a predetermined genetic locus to ensure uniformity of the insertion site of the transgene(s) in the genome and limit the number of integration events. Site-specific integration aided by, for example, adeno-associated virus vector (AAV), can also enable expression of more than one gene, while maintaining high transduction efficiency.

An SSI approach aided by AAV was used to deliver three different constructs to generate three different types of engineered cells for use in these experiments: 1) a CD 19 CAR construct, 2) a CD19 CAR/CD70 binding protein construct, and 3) a CD70 CAR construct. For construct 2), a CD70 binding protein (or domain) was derived from an anti-CD70 antibody clone 4F11 in the form of an scFv and a CD3C signaling domain (4F11z). In each case, the TRAC locus was targeted for integration of the construct. Three different PBMC donors were used. Expression of each construct was driven by a PGK promoter. Further gene editing was performed via CRISPR/Cas9 in 1) and 2) to generate graft CAR T cells with certain knockouts: TRAC knockout only, TRAC/B2M knockout, TRAC/CD58 knockout. After a 1-week in vitro expansion, target cells were then purified to remove any TCR α/β expressing cells. The level of specific gene knockout or MHC-I and MHC-II knockdown was evaluated by flow cytometry. PBMCs from HLA-A2-expressing (HLA-A2+) healthy human donors were used as allogeneic effectors (host), while the engineered cells were used as graft CAR T cells.

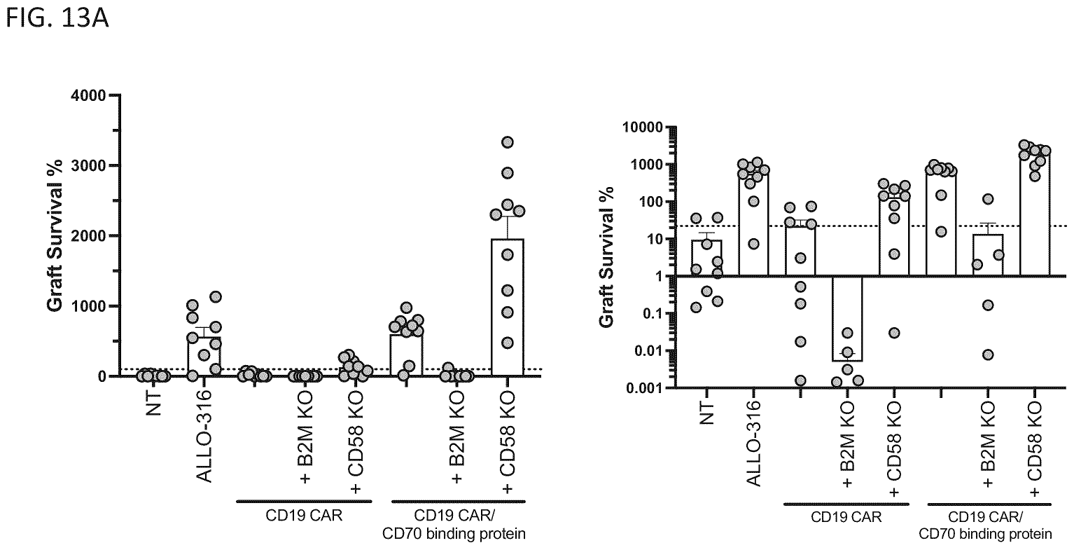

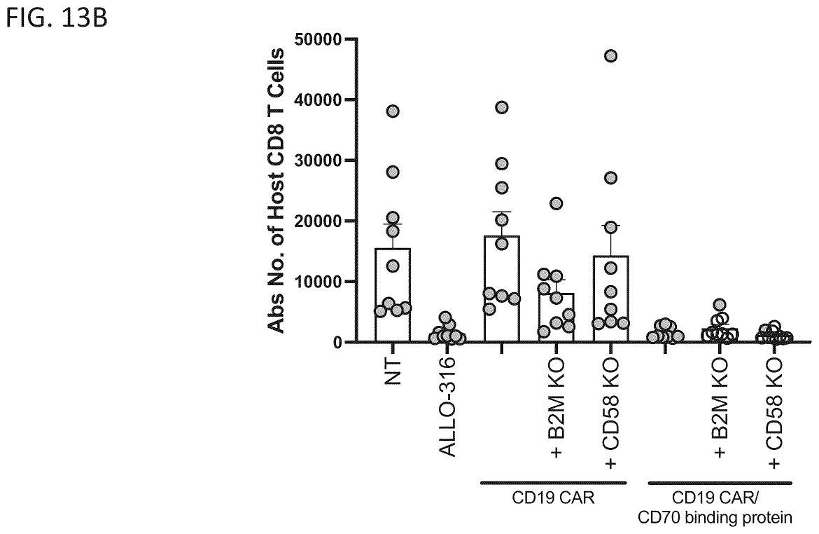

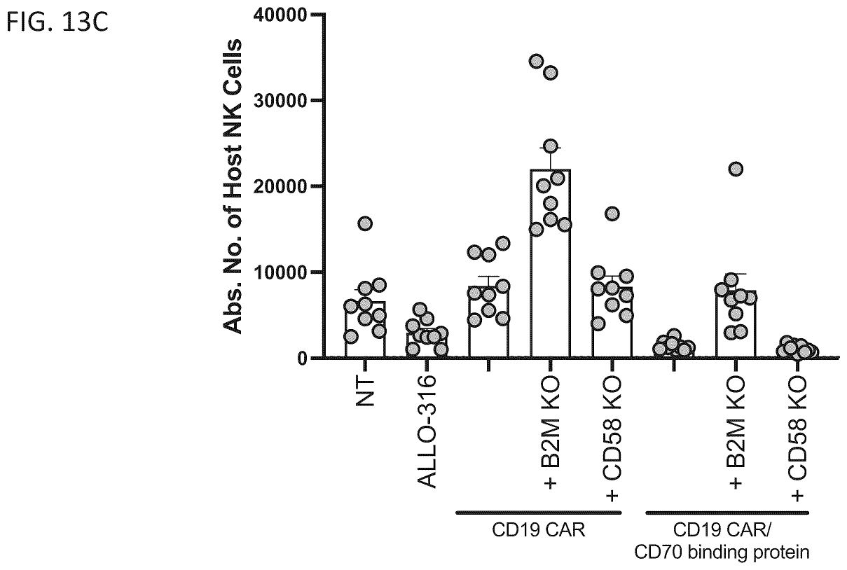

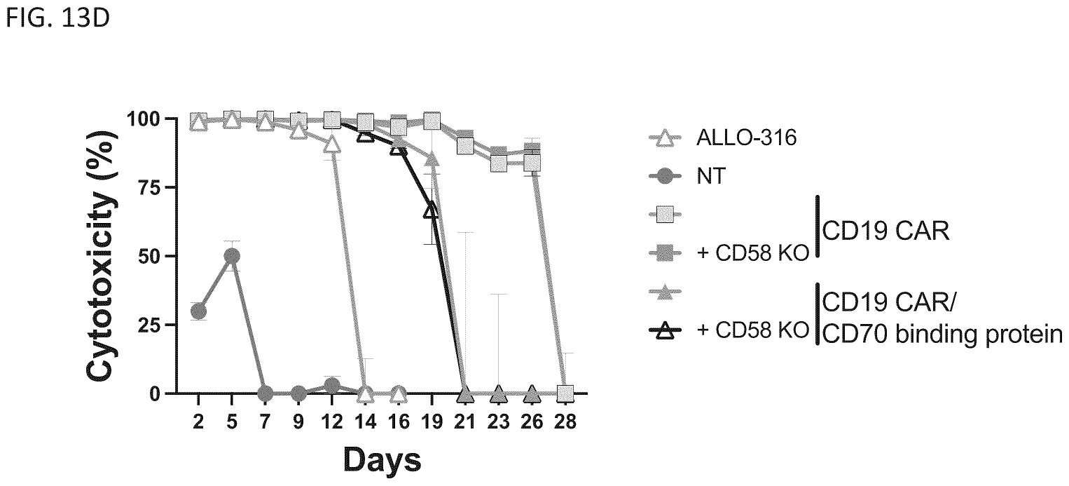

PBMC MLR assays were performed. A ratio of 10:1 host:graft cells was used for a 13-day culture. PBMCs from 3 different donors were depleted of B cells prior to co-culture. Readouts were performed on day 13. As shown in FIG. 13A, the CD58 knockout was found to enhance survival of the CD19 CAR/CD70 binding protein CAR T cells (each symbol represents 1 of 9 unique graft/host pairs; each symbol shape represents a unique graft).

The ability of the engineered CAR T cells to impact the expansion of host immune cells after 9 days in co-culture was evaluated. As shown in FIG. 13B, the CD19 CAR/CD70 binding protein CAR T cells were found to reduce expansion or deplete host CD8+ T cells. Similar results were observed for host CD4+ T cells. As shown in FIG. 13C, the CD19 CAR/CD70 binding protein CAR T cells were observed to prevent host NK cell expansion.

In vitro long-term killing assays were performed using the various CAR T knockout cells against target Raji WT cells, at a E:T ratio of 4:1. As shown in FIG. 13D, the introduction of a CD58 knockout did not decrease the cytotoxic activity of the CAR T cells. CAR T cells were co-cultured with luciferase-GFP-expressing Raji cells at an effector-to-target ratio of 8:1. Every 2-3 days, half the cells were passaged onto fresh Raji cells. The remaining half of cells was then used to determine target cell killing using Bright-glo reagent (Promega).

### General Protocols

Target cell generation (for FIG. 1) Primary human T cells were isolated from frozen healthy donor peripheral blood mononuclear cells (PBMCs) using magnetic-activated cell sorting (MACS) negative selection (Miltenyi, human pan T cell isolation kit, Cat #130-096-535) and activated with 1:100 (v:v) T cell TransAct (Miltenyi, Cat #130-111-160)+100 IU/mL IL-2 (Miltenyi, Cat #130-097-746) in R10 (RPMI-1640+10% FBS+25 mM HEPES+Sodium Pyruvate+non-essential amino acids). After 2 days, T cells were gene edited using the Neon Transfection System (Invitrogen). Briefly, ribonucleoprotein (RNP) complexes were generated by mixing cas9 enzyme (IDT, Cat #1081059) and sgRNA at a 2:1 molar ratio for 10 min at room temperature. If two sgRNAs were used, incubation was performed with cas9 at a 1:1:1 ratio (sgRNA1: sgRNA2: cas9). If three sgRNAs were used, incubation was performed with cas9 at a 0.67:0.67:0.67:1 ratio (sgRNA1: sgRNA2: sgRNA3: cas9). Cells were pulsed at 1600 V, 10 ms width for a total of 3 times and immediately recovered in R10 media supplemented with 100 IU/mL IL-2+10 ng/mL IL-7 (Miltenyi, Cat #130-095-363). Edited T cells were incubated at 37° C., 5% CO₂. Fresh R10 with 100 IU/mL IL-2 was added the next day to the cells. After 5-7 days post gene editing, KO efficiency was assessed via flow cytometry. KO efficiency was assessed in various ways: For TRAC, CD3/TCRab expression was assessed; for β2m, NLRC5 and RFX5, anti-β2m or an anti-HLA antibody was used. anti-CD58 antibody was used to assess CD58 expression. TRAC KO cells were purified using MACS negative selection (Stem Cell Technologies, EasySep human TCR alpha/beta depletion kit, Cat #17847) according to the manufacturer's recommendations. Purified T cells were used immediately or frozen at 5e6 cell aliquots in 90% FBS+10% DMSO.

Target cell production (for all figures except FIG. 1E-F) Primary human T cells were isolated from frozen healthy donor peripheral blood mononuclear cells (PBMCs) using EasySep Human T cell Isolation Kit (StemCell Technologies, Cat #17951). Isolated T cells were activated with 1:100 (v:v) T cell TransAct (Miltenyi, Cat #130-111-160)+100 IU/mL IL-2 (Miltenyi, Cat #130-097-746) in X-Vivo 15 medium (Lonza, Cat #04-418Q)+5% Human AB Serum (Gemini, Cat #100-318). After 2 days, T cells were gene edited using the Nucleofector 4D system (Lonza). Briefly, ribonucleoprotein (RNP) complexes were generated by mixing cas9 enzyme (IDT, Cat #1081059) and sgRNA at a 1:1 molar ratio for 10 min at room temperature. If two sgRNAs were used, incubation was performed with cas9 at a 0.5:0.5:1 ratio (sgRNA1: sgRNA2: cas9). If three sgRNAs were used, incubation was performed with cas9 at a 0.3:0.3:0.3:1 ratio (sgRNA1: sgRNA2: sgRNA3: cas9). Immediately after electroporation, cells were recovered in X-Vivo 15 medium+5% Human AB Serum and 100 IU/mL IL-2. In some cases (FIGS. 5-6), electroporated T cells were transduced with an adenovirus for the expression of a CD19 CAR (FMC63 TurboCAR). TCR depletion (Stem Cell Technologies, EasySep human TCR alpha/beta depletion kit, Cat #17847) was typically performed between days 14-16. TCR-depleted T cells were cryopreserved and thawed for additional assays at later times. Cell expansion during production was tracked by counting viable cells with Vi-CELL counter (Beckman Coulter). Fold expansion was calculated by comparing viable cell counts at various time points to day 2 (time of gene editing). CD19 CAR T cells were identified by anti-idiotypic antibody (Acro Biosystems, Cat #FM3HPY53). KO efficiency was also assessed via flow cytometry in multiple timepoints throughout cell production. KO efficiency was assessed in various ways: For TRAC, CD3/TCRab expression was assessed; for β2m, NLRC5 and RFX5, an anti-HLA-ABC antibody was used. anti-CD58, anti-CD48, and anti-ICAM-1 antibodies were used to assess CD58, CD48, and ICAM-1 KO efficiency, respectively. TRAC KO cells were purified using MACS negative selection (Stem Cell Technologies, EasySep human TCR alpha/beta depletion kit, Cat #17847) according to the manufacturer's recommendations. Purified T cells were frozen at 5e6 cell aliquots in CryoStor CS5 medium (Stem Cell Technologies, Cat #07933).

Primed T MLR (FIGS. 1E-F, 3, and 5) HLA-A2+ Human PBMCs were primed against irradiated PBMCs or pan T cells derived from donors (HLA-A2−) used to make target T cells above to promote expansion of alloreactive T cell clones. Briefly, target PBMCs were irradiated at 30 Gy and co-cultured with host PBMCs at a 1:1 ratio in R10+20 IU/mL IL-2+10 ng/mL TL-7+10 ng/mL IL-15 (Miltenyi, Cat #130-095-765) for 4 days. Media was exchanged to R10 without cytokines and the cells were continued to culture for 3 more days. Afterwards, pan T cells were isolated using MACS negative selection (Miltenyi, human pan T cell isolation kit, Cat #130-096-535) per the manufacturer's recommendations. In a 96-well plate, 20,000 target T cells were seeded with 20,000 primed host T cells and cultured in R10+20 IU/mL IL-2 for 2 days at 37° C., 5% CO₂. In some cases (FIG. 5), 5,000 GFP-expressing β2m KO Raji tumor cells were added to the co-culture to activate CD19 CAR T graft cells. Survival of graft T cells was determined by flow cytometry using absolute counts by gating on live GFP-HLA-A2-TCRαβ− CD4+CD8+(CAR+) and desired gene edits (e.g., CD58 KO).

NK MLR (FIGS. 1F and 6). FIG. 1B: Human NK cells were isolated from freshly isolated human HLA-A2+ PBMCs via MACS purification (Miltenyi, human pan NK cell isolation kit, Cat #130-092-657). In a 96-well plate, 20,000 HLA-A2− graft T cells were seeded with 20,000 or 100,000 host NK cells, for effector:target=1:1 or 5:1, and cultured in R10+1000 IU/mL IL-2 for 2 days at 37° C., 5% CO₂. Survival of graft T cells was determined by flow cytometry using absolute counts by gating on live HLA-A2-TCRαβ− CD56− CD4+CD8+ and desired gene edits (e.g., CD58 KO).

FIG. 6: Human NK cells were isolated from frozen human HLA-A2+ PBMCs via MACS purification (StemCell Technologies, EasySep human NK cell isolation kit, Cat #100-0960). In a 96-well plate, 20,000 HLA-A2− graft T cells were seeded with 200,000 host NK cells, for effector:target=10:1, and cultured in R10+1000 IU/mL IL-2 for 2 days at 37° C., 5% CO₂. 5,000 GFP-expressing Raji tumor cells were added to the co-culture to stimulate CD19 CAR T graft cells. Survival of graft T cells was determined by flow cytometry using absolute counts by gating on live GFP-HLA-A2-TCRαβ− CD56− CD4+CD8+(CAR+) and desired gene edits (e.g., CD58 KO).

PBMC MLR (FIG. 4) In a 96-well plate, 20,000 HLA-A2− graft T cells were seeded with 200,000 HLA-A2+ host PBMC cells (10:1 effector:target ratio) and cultured in R10+20 IU/mL IL-2 for 9 days at 37° C., 5% CO₂. Cells were fed every 3-4 days with fresh R10+IL-2. Survival of graft T cells was determined by flow cytometry by gating on live HLA-A2-TCRαβ− CD56− CD4+CD8+ and desired gene edits (e.g., CD58 KO).

### General Methods

Generation of KO T Cells

Primary human T cells were isolated from frozen healthy donor peripheral blood mononuclear cells (PBMCs) using magnetic-activated cell sorting (MACS) negative selection (Miltenyi, human pan T cell isolation kit, Cat #130-096-535) and activated with 1:100 (v:v) T cell TransAct (Miltenyi, Cat #130-111-160)+100 IU/mL IL-2 (Miltenyi, Cat #130-097-746) in R10 (RPMI-1640+10% FBS+25 mM HEPES+Sodium Pyruvate+non-essential amino acids). After 2 days, T cells were gene edited using the Neon Transfection System (Invitrogen). Briefly, ribonucleoprotein (RNP) complexes were generated by mixing cas9 enzyme (IDT, Cat #1081059) and sgRNA at a 1:1 molar ratio for 10 min at room temperature. If two sgRNAs were used, incubation was performed with cas9 at a 0.5:0.5:1 ratio (sgRNA1: sgRNA2: cas9). Cells were pulsed at 1600 V, 10 ms width for a total of 3 times and immediately recovered in R10 media supplemented with 100 IU/mL IL-2+10 ng/mL IL-7 (Miltenyi, Cat #130-095-363). Edited T cells were incubated at 37° C., 5% CO₂. Fresh R10 with 100 IU/mL IL-2 was added the next day to the cells. After 5-7 days post gene editing, KO efficiency was assessed via flow cytometry. KO efficiency ranged from around 50% to as high as about 100%. KO efficiency was assessed in various ways: For TRAC, CD3/TCRab expression was assessed; for β2m, NLRC5 and RFX5, anti-β2m or an anti-HLA antibody was used. Lower editing efficiencies are acceptable because edited cells are purified as described below. KO was checked at about day 22 and was found to be relatively stable.

TRAC KO cells were purified using MACS negative selection (Stem Cell Technologies, EasySep human TCR alpha/beta depletion kit, Cat #17847) according to the manufacturer's recommendations. Purified T cells were used immediately or frozen at 5e6 cell aliquots in 90% FBS+10% DMSO.

TABLE 4.2
List of sgRNA sequences

| SEQ ID | Gene used | sgRNA sequence |
|---|---|---|
| NO: | for | (5′-3′) |
| 91 | TRAC | gcugguacacggcaggguca |
| 92 | B2M (β2m) | ucacgucauccagcagagaa |
| 93 | NLRC5 | cagcucugcaaggcucuggg |
| 94 | RFX5 | aguacauguaugccuauagg |
| 98 | CIITA | guggcacacugugagcugcc |
| 99 | RFXANK (sgRNA 1) | uuguugacgagguugucacc |
| 100 | RFXANK (sgRNA 2) | gcucgucuggcuuguugacg |
| 101 | RFXANK (sgRNA 3) | gucaacaagccagacgagcg |
| 102 | RFXAP (sgRNA 1) | ucuuagcagcauggaggcgc |
| 103 | RFXAP (sgRNA 2) | cuuagcagcauggaggcgca |
| 104 | RFXAP (sgRNA 3) | cauggaggcgcaggguguag |
| 105 | TAP2 | cgcguccaccagcagcaggg |

### Example 5—T Cell Rejection in an NSG Mouse Model

Persistence and anti-tumor efficacy of CAR T cells (e.g., CD19 CAR T cells as described herein) with various gene knockouts (KG) can be assessed in an allogeneic T cell rejection model in vivo. Suitable mice, e.g., 8-12 week old NOD.Cg-Prkdcscid 1l2rgtm1Wj1/SzJ (NSG) mice, are obtained from Jackson Laboratories (Bar Harbor, ME). NSG mice are irradiated (e.g., at 1 Gy on day −6) and receive a suitable number of allogeneic human T cells, e.g., 7×10⁶ in vitro expanded allogeneic human T cells derived from an HLA-A2+ recipient. Briefly, allogeneic T cells are activated immediately after recovery from cryopreservation, e.g., using T cell TransAct™ (Miltenyi Biotec, Auburn, CA; 1:100 dilution), in X-Vivo 15 medium (Lonza, Basel, Switzerland) supplemented with 5% human AB serum (Gemini Bio-Products, West Sacramento, CA) and 100 IU/mL IL-2 (Miltenyi Biotec, Auburn, CA). Two days after activation, T cells are centrifuged to remove TransAct, and then are resuspended, e.g., in fresh XVivo-15 medium supplemented with 5% human AB serum and 100 IU/ml IL-2 for 6-8 days before animal dosing, via intravenous injection. On day −4, mice receive cells expressing the CAR T target, e.g., 1×10⁵ luciferase-labeled B2M KO Raji tumor cells via intravenous injection. On day −1, mice are randomized based on total body bioluminescence. On day 0, CD19 CAR T cells produced from an HLA-A2− donor with various gene editing modifications (e.g., TRAC KO alone or with a KO of one or more of CD48, CD58, ICAM-1, β2m, NLRC5 and RFX5) are intravenously injected with a suitable number of cells, e.g., 3×10⁶ CAR′ cells. Total T cell numbers are kept constant across all groups by normalizing with non-transduced T cells. Growth of Raji tumor are tracked by total body bioluminescence at indicated time points. To track CAR T cell persistence in vivo, peripheral blood is obtained, e.g., 40 μL of peripheral blood is obtained by cheek-bleeding on day 2, 9, and 16 post CAR T dosing. After red blood cell lysis, samples are stained with antibodies, e.g., anti-human CD45, HLA-A2, and anti-idiotypic antibodies, to identify CD19 CAR T cells. Counting beads (123count eBeads, Thermo Fisher) are used to normalize CAR T cell counts. N=8 mice per group.

### Example 6 NK Rejection in a Syngeneic Mouse Model

Production of gene-edited mouse T cells: Spleens of C57BL/6 mice with CD45.2 alleles are harvested, and single cell suspension is obtained, e.g., by gentleMAC Dissociator (Miltenyi). Mouse T cells are then purified, e.g., with the mouse T cell isolation kit II and subsequently activated by mouse T cell activation/expansion kit, following manufacturer's protocols (Miltenyi). After activation, e.g., one day post activation, mouse T cells are gene edited, e.g., with CRISPR/Cas9 via electroporation (Nucleofector 4D, Lonza) similarly as human T cells. Exemplary sgRNA sequence(s) that are used are listed in Table 4.1 or 4.2 above. Gene-edited mouse T cells are expanded in vitro under suitable conditions, e.g., for 6 days in the presence of IL-2 (40 ng/ml) and IL-7 (40 ng/ml), before animal dosing.

Adoptive transfer of graft T cells: suitable mice, e.g., 8-12 week old C57BL/6 mice with CD45.1 or CD45.2 alleles, are obtained from Jackson Laboratories (Bar Harbor, ME). C59BL/6 mice with CD45.1 alleles are irradiated, e.g., at 2.5 Gy on day −3, and receive 1×10⁷ control (non-gene-edited) or gene-edited (a KO of one or more of CD48, CD58, ICAM-1, β2m, NLRC5 and RFX5) in vitro expanded T cells from C57BL/6 mice, e.g., expanded T cells with CD45.2 alleles, via intravenous injection on day 0. Some C57BL/6 mice with CD45.1 alleles also receive NK depleting antibody (anti-NK1.1 antibody, BioXcell) on days −3 and 0 via intraperitoneal injection, at 200 ug/mouse for each injection, and are indicated as a “+NK depletion” cohort. Cell counts of host immune cells and graft T cells in the peripheral blood are tracked by flow cytometry. Samples, e.g., 40 μL of peripheral blood, are obtained by cheek bleeding on indicated time points post graft T cell dosing. After red blood cell lysis, samples are stained with anti-mouse antibodies for CD45.1, CD45.2, CD3, CD4, CD8, CD19, CD335, CD11b, and H2-K^{b} to identify graft T cells and host immune cell subsets. Counting beads (123count eBeads, Thermo Fisher) are used to normalize cell counts. N=5 mice per group. Mean±SEM shown.

Sublethal irradiation is used to temporarily reduce host immune cells of C57BL/6 mice before graft dosing. Some mice also receive NK depleting antibody to deeply deplete residual NK cells. Gene-edited (one or more KOs as described herein) or un-edited control graft T cells from syngeneic mice are then adoptively transferred to the host. Engraftment of the adoptive transferred T cells is tracked at various time points by flow cytometry.

All references cited herein, including patents, patent applications, papers, text books, and the like, and the references cited therein, to the extent that they are not already, are hereby incorporated by reference in their entirety.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

What is claimed is:

1. An engineered immune cell comprising a genomic modification that functionally impairs or reduces expression of (i) RFX5 and/or NLRC5 and (ii) CD58 relative to a cell without the genomic modification.

2. The engineered immune cell of claim 1, wherein the genomic modification comprises a knockdown and/or a knockout of (i) RFX5 and/or NLRC5 and/or (ii) CD58.

3. The engineered immune cell of claim 1 or 2, wherein the genomic modification comprises one or more modifications at the gene locus of (i) RFX5 and/or NLRC5 and (ii) CD58.

4. The engineered immune cell of any one of claims 1-3, wherein the genomic modification comprises a deletion or an insertion at the gene locus of (i) RFX5 and/or NLRC5 and (ii) CD58.

5. The engineered immune cell of any one of claims 1-4, wherein the genomic modification is selected from the group consisting of (i) an insertion of one or more nucleotides, (ii) an insertion of a polynucleotide sequence that encodes a protein, (iii) a deletion of one or more nucleotides, and (iv) a substitution of one or more nucleotides.

6. The engineered immune cell of any one of claims 1-5, wherein the genomic modification was introduced by a gene editing technology selected from TALEN, zinc finger, Cas-CLOVER, and a CRISPR/Cas system.

7. The engineered immune cell of claim 1, wherein the genomic modification comprises an insertion of an RNA interference sequence.

8. The engineered immune cell of claim 7, wherein the RNA interference sequence is an shRNA sequence, an siRNA sequence, or a miRNA sequence.

9. The engineered immune cell of claim 7 or 8, wherein the RNA interference sequence comprises a sequence that is complementary to (i) RFX5 and/or NLRC5 and/or (ii) CD58 gene sequences.

10. The engineered immune cell of any one of claims 1-9, which further comprises a polynucleotide sequence encoding an antigen binding protein and/or a CD70 binding protein.

11. The engineered immune cell of claim 10, wherein the antigen binding protein is a chimeric antigen receptor (CAR) or a T cell receptor (TCR).

12. The engineered immune cell of any one of claims 1-11, wherein the cell is further engineered to comprise one or more genomic modifications that functionally impair or reduce expression of one or more of ICAM-1, TAP2, β2m, TRAC, CIITA, RFXAP, RFXANK, and CD48 relative to a non-engineered cell.

13. The engineered immune cell of any one of claims 1-12, wherein the engineered immune cell has improved persistence and/or improved resistance against alloreactive immune cell rejection as compared to an immune cell that does not comprise the genomic modification.

14. The engineered immune cell of claim 13, wherein the alloreactive immune cell rejection comprises alloreactive T cell-mediated rejection and/or alloreactive natural killer (NK) cell-medicated rejection.

15. The engineered immune cell of any one of claims 1-14, which comprises one or more genomic modifications that functionally impair or reduce expression of RFX5 and CD58.

16. The engineered immune cell of any one of claims 1-14, which comprises one or more genomic modifications that functionally impair or reduce expression of NLRC5 and CD58.

17. The engineered immune cell of any one of claims 1-14, which comprises one or more genomic modifications that functionally impair or reduce expression of RFX5, NLRC5, and CD58.

18. The engineered immune cell of any one of claim 1-17, wherein β2m is functionally expressed at a reduced level.

19. The engineered immune cell of any one of claims 1-17, which comprises an unmodified β2m gene, or wherein β2m is not functionally expressed at a reduced level.

20. The engineered immune cell of claim 13, wherein the increased persistence is determinable and/or determined by a mixed lymphocyte reaction (MLR) assay.

21. The engineered immune cell of claim 13, wherein the improved resistance against alloreactive immune cell rejection is determinable and/or determined by an MLR assay.

22. The engineered immune cell of any one of claims 1-21, wherein the engineered immune cell exhibits:
(i) a reduced level of expression of an MHC class I protein or complex at the cell surface,
(ii) a reduced level of expression of an MHC class II protein or complex at the cell surface, or
(iii) a reduced level of expression of an MHC class I protein or complex at the cell surface and a reduced level of expression of an MHC class II protein or complex at the cell surface.

23. The engineered immune cell of claim 10 or 11, wherein the antigen binding protein is a CAR.

24. The engineered immune cell of claim 10 or 11, which expresses the antigen binding protein and/or the CD70 binding protein.

25. The engineered immune cell of claim 10, wherein the polynucleotide sequence encoding the antigen binding protein and/or the CD70 binding protein is located within a disrupted CD58, RFX5, NLRC5, ICAM-1, CD48, TAP2, β2m, TRAC, CIITA, RFXAP or RFXANK locus.

26. The engineered immune cell of any one of claims 1-25, wherein the engineered immune cell further comprises one or more genomic modifications of an endogenous TCRa gene.

27. The engineered immune cell of claim 26, wherein the engineered immune cell further comprises one or more genomic modifications of an endogenous CD52 gene.

28. The engineered immune cell of any one of claims 1-27, wherein the engineered immune cell is or is obtained from an immune cell of a healthy volunteer, is obtained from a patient, or is obtained from an induced pluripotent stem cell (iPSC).

29. The engineered immune cell of any one of claims 1-27, wherein the engineered immune cell is not a natural killer (NK) cell or is not obtained from an NK cell of a healthy volunteer or patient.

30. The engineered immune cell of any one of claims 1-27, wherein the engineered immune cell is not obtained from an iPSC.

31. A population of engineered immune cells comprising the engineered cells according to any one of claims 1-30, wherein no more than 50% of the engineered immune cells functionally express (i) RFX5 and/or NLRC5 and (ii) CD58.

32. The population of claim 31, wherein no more than 50% of the engineered immune cells functionally express RFX5 and CD58.

33. The population of claim 31, wherein no more than 50% of the engineered immune cells functionally express NLRC5 and CD58.

34. The population of claim 31, wherein no more than 50% of the engineered immune cells functionally express RFX5, NLRC5, and CD58.

35. The population of any one of claims 31-34, wherein no more than 50% of the engineered immune cells also functionally express
a) any one or more of ICAM-1, CD48, TAP2, β2m, TRAC, CIITA, RFXAP and RFXANK; or
b) only one of ICAM-1 and CD48; or
c) both CD48 and ICAM-1.

36. The population of any one of claims 31-35, wherein an engineered immune cell of the population of engineered immune cells has improved persistence and/or improved resistance against alloreactive immune cell rejection as compared to a non-engineered immune cell,
optionally wherein the improved resistance is against alloreactive T cell-mediated rejection and/or alloreactive natural killer (NK)-mediated rejection, and
optionally wherein the increased persistence is determinable and/or determined by a mixed lymphocyte reaction (MLR) assay and/or wherein the improved resistance against alloreactive immune cell rejection is determinable and/or determined by an MLR assay.

37. A population of engineered immune cells comprising engineered immune cells according to any one of claims 1-30, wherein at least 1% of the engineered immune cells functionally express (i) RFX5 and/or NLRC5 and (ii) CD58 at a level not greater than 50% of the expression level in non-engineered immune cells.

38. The population of claim 37, wherein at least 1% of the engineered immune cells functionally express RFX5 and CD58 at a level not greater than 50% of the expression level in non-engineered immune cells.

39. The population of claim 37, wherein at least 1% of the engineered immune cells functionally express NLRC5 and CD58 at a level not greater than 50% of the expression level in non-engineered immune cells.

40. The population of claim 37, wherein at least 1% of the engineered immune cells functionally express RFX5, NLRC5, and CD58 at a level not greater than 50% of the expression level in non-engineered immune cells.

41. The population of claim 37, wherein at least 1% of the engineered immune cells functionally express a) any one or more of ICAM-1, CD48, TAP2, β2m, TRAC, CIITA, RFXAP and RFXANK; or
b) only one of ICAM-1 and CD48; or
c) both CD48 and ICAM-1,
at a level not greater than 50% of the expression level in non-engineered immune cells.

42. The population of any one of claims 37-41, wherein an engineered immune cell of the population of engineered immune cells has improved persistence and/or improved resistance against alloreactive immune cell rejection as compared to a non-engineered immune cell,
optionally wherein the improved resistance is against alloreactive T cell-mediated rejection and/or alloreactive natural killer (NK)-mediated rejection, and
optionally wherein the increased persistence is determinable and/or determined by a mixed lymphocyte reaction (MLR) assay and/or wherein the improved resistance against alloreactive immune cell rejection is determinable and/or determined by an MLR assay.

43. The population of engineered immune cells of any one of claims 31-42, wherein at least 50% of the engineered immune cells exhibit a reduced level of expression of an MHC class I protein or complex at the cell surface.

44. The population of engineered immune cells of any one of claims 31-43, wherein the population of engineered immune cells comprises at least 10% engineered T cells, at least 20% engineered T cells, at least 30% engineered T cells, at least 40% engineered T cells, at least 50% engineered T cells, at least 75% engineered T cells, or at least 90% engineered T cells.

45. The population of engineered immune cells of any one of claims 31-44, wherein at least 10%, at least 20%, at least 30%, at least 40%, at least 50%, at least 75%, or at least 90% of the engineered immune cells further express an antigen binding protein or a CD70 binding protein.

46. The population of engineered immune cells of claim 45, wherein the antigen binding protein is a CAR or a TCR.

47. The population of engineered immune cells of claim 45, wherein a nucleic acid encoding the antigen binding protein and/or the CD70 binding protein is inserted into a disrupted locus of CD58, RFX5, NLRC5, CD48, ICAM-1, TAP2, NLRC5, β2m, TRAC, RFX5, RFXAP, CIITA and RFXANK.

48. The population of engineered immune cells of claim 46, wherein a nucleic acid encoding the CAR is inserted into a disrupted locus of CD58, RFX5, NLRC5, CD48, ICAM-1, TAP2, NLRC5, β2m, TRAC, RFX5, RFXAP, CIITA and RFXANK.

49. The population of engineered immune cells of claim 46, wherein a nucleic acid encoding the TCR is inserted into a disrupted locus that is not a disrupted locus of CD58, RFX5, NLRC5, CD48, ICAM-1, TAP2, NLRC5, β2m, TRAC, RFX5, RFXAP, CIITA and RFXANK.

50. The population of engineered immune cells of any one of claims 31-49, wherein at least 10%, at least 20%, at least 30%, at least 40%, at least 50%, at least 75%, or at least 90% of the engineered cells further comprises one or more genomic modifications of one or more of an endogenous TCRa gene and an endogenous CD52 gene.

51. The population of engineered immune cells of any of claims 31-50, wherein at least 10%, 20%, 30%, 40%, 50%, 75%, or 90% of the engineered immune cells further express one or more proteins selected from the group consisting of HLA-E, HLA-E single-chain trimer, HLA-G or HLA-G single-chain trimer, UL18 or UL18 single-chain trimer, HLA-A2 and HLA-A2 single-chain trimer.

52. The population of engineered immune cells of any one of claims 31-51, wherein the functional expression level of one or more of TAP2, NLRC5, β2m, TRAC, RFX5, RFXAP, CIITA and RFXANK is measured by determining the surface expression level of HLA, beta2 microglobulin (B2M) or both HLA and B2M on the surface of the engineered immune cell or is measured by flow cytometry.

53. The population of engineered immune cells of any one of claims 31-51, wherein the functional expression level of one or more of CD48, CD58, and ICAM-1 is measured by determining the surface expression level of one or more of CD48, CD58, and ICAM-1 on the surface of the engineered immune cell or is measured by flow cytometry.

54. A method of making the engineered immune cell of any one of claims 1-30 or a population of any one of claims 31-53 comprising
a) modifying the genome of an engineered immune cell, and
b) producing the engineered immune cell that comprises genomic modifications.

55. The method of claim 54, wherein the genome of the engineered immune cell is modified using TALEN, zinc finger, Cas-CLOVER, or a CRISPR/Cas system.

56. A pharmaceutical composition comprising the engineered immune cell of any one of claims 1-30, the population of engineered immune cells of any one of claims 31-53, or the engineered immune cell made by the method of claim 54 or 55, and further comprising at least one pharmaceutically acceptable carrier or excipient, and optionally
wherein the engineered immune cell or one or more cells of the population (i) further express one or more proteins selected from the group consisting of HLA-E, HLA-E single-chain trimer, HLA-G, HLA-G single-chain trimer, UL18, UL18 single-chain trimer, HLA-A2, HLA-A2 single-chain trimer, and human cytomegalovirus (HCMV) US 11, and/or (ii) is further engineered to not express or to express at a reduced level any one or more of TAP2, NLRC5, β2m, TRAC, CIITA, RFXANK, RFXAP and RFX5.

57. A method of treating a condition in a subject comprising administering to the subject: the engineered immune cell of any one of claims 1-30, the population of engineered immune cells of any one of claims 31-53, the engineered immune cell made by the method of claim 54 or 55, or the pharmaceutical composition of claim 56.

58. The method of claim 57, wherein the condition is a solid tumor or a liquid tumor.

59. A method of improving (i) persistence or (ii) resistance against an alloreactive immune cell rejection of allogeneic engineered immune cells comprising
a) modifying allogeneic immune cells to introduce one or more genomic modifications that functionally impair or reduce expression of (i) RFX5 and/or NLRC5 and (ii) CD58 to provide allogeneic engineered immune cells; and
b) administering the allogeneic engineered immune cells to a subject.

60. The method of claim 59, wherein the genomic modifications comprise a knockdown and/or a knockout of (i) RFX5 and/or NLRC5 and (ii) CD58.

61. The method of claim 59 or 60, wherein the genomic modifications comprise one or more modifications at the gene locus of (i) RFX5 and/or NLRC5 and (ii) CD58.

62. The method of any one of claims 59-61, wherein the genomic modifications comprise a deletion or an insertion at the gene locus of (i) RFX5 and/or NLRC5 and (ii) CD58.

63. The method of any one of claims 59-62, wherein the genomic modifications are selected from the group consisting of (i) an insertion of one or more nucleotides, (ii) an insertion of a sequence that encodes a protein, (iii) a deletion of one or more nucleotides, and
(iv) a substitution of one or more nucleotides.

64. The method of any one of claims 59-63, wherein the genomic modifications were introduced by a gene editing technology selected from TALEN, zinc fingers, Cas-CLOVER, and a CRISPR/Cas system.

65. The method of claim 59, wherein the genomic modifications comprise an insertion of an RNA interference sequence.

66. The method of claim 65, wherein the interference sequence is an shRNA sequence, an siRNA sequence, or a miRNA sequence.

67. The method of claim 65 or 66, wherein the interference sequence comprises a sequence that is complementary to (i) RFX5 and/or NLRC5 and (ii) CD58 gene sequences.

68. The method of any one of claims 59-67, wherein the allogeneic engineered immune cells further comprise a polynucleotide sequence encoding an antigen binding protein and/or a CD70 binding protein.

69. The method of claim 68, wherein the antigen binding protein is a CAR or a TCR.

70. The method of any one of claims 59-69, wherein the allogeneic engineered immune cells are further engineered to comprise one or more genomic modifications that functionally impair or reduce expression of one or more of ICAM-1, TAP2, β2m, TRAC, CIITA, RFXAP, RFXANK, and CD48 relative to cells without the modification.

71. The method of any one of claims 59-70, wherein the genomic modifications functionally impair or reduce expression to about 50% or less of the corresponding level in cells without the genomic modifications.

72. The method of any one of claims 59-71, wherein the allogeneic engineered immune cells have improved persistence and/or improved resistance against an alloreactive immune cell rejection as compared to allogeneic non-engineered immune cells.

73. The method of any one of claims 59-72, wherein the improved resistance is against alloreactive T cell-mediated rejection and/or alloreactive natural killer (NK)-mediated rejection.

74. The method of any one of claim 59-73, wherein the increased persistence is determinable and/or determined by a mixed lymphocyte reaction (MLR) assay and/or wherein the improved resistance against alloreactive immune cell rejection is determinable and/or determined by an MLR assay.

75. The method of any one of claims 59-74, wherein the method comprises functionally impairing or reducing expression of RFX5 and CD58.

76. The method of any one of claims 59-74, wherein the method comprises functionally impairing or reducing expression of NLRC5 and CD58.

77. The method of any one of claims 59-74, wherein the method comprises functionally impairing or reducing expression of RFX5, NLRC5, and CD58.

78. The method of any one of claims 59-77, wherein the extent of reduction in the expression level of (i) RFX5 and/or NLRC5 and (ii) CD58 is determined relative to the expression level of (i) RFX5 and/or NLRC5 and (ii) CD58, respectively, in a cell of the same type that has not been modified.

79. The method of any one of claims 59-78, wherein the functional expression level of one or more of CD48, CD58, and ICAM-1 is measured by determining the surface expression level of a CD48 protein, a CD58 protein or an ICAM-1 protein on the surface of the engineered immune cell.

80. The method of any one of claims 59-79, wherein the functional expression level of RFX5 and/or NLRC5 is measured by determining the surface expression level of HLA, beta2 microglobulin (B2M) or both HLA and B2M on the surface of the engineered immune cell.

81. The method of claim 79 or 80, wherein the surface expression level is measured by flow cytometry.

82. The method of any one of claims 59-81, wherein the method further comprises introducing one or more genomic modifications of one or more of a TCRa gene and a CD52 gene.

83. The method of any one of claim 59-82, wherein the allogeneic engineered immune cells comprise an unmodified β2m gene or wherein β2m is not functionally expressed at a reduced level in the allogeneic engineered immune cell.

84. The engineered immune cell of any one of claims 20-30, which comprises an unmodified β2m gene or wherein β2m is not functionally expressed at a reduced level in the engineered immune cell.

85. The engineered immune cell of any one of claim 1-30, wherein the expression of one or more of β2m, RFX5, NLRC5, CIITA, and TAP2 is not functionally impaired or reduced.

86. The population of engineered immune cells of any one of claims 31-53, which do not comprise genomic modifications of one or more of β2m, RFX5, NLRC5, CIITA, or TAP2 gene.

87. The population of engineered immune cells of any one of claims 31-53, wherein the expression of one or more of β2m, RFX5, NLRC5, CIITA, or TAP2 is not functionally impaired or reduced.

88. The method of claim 54 or 55, wherein the genomic modifications do not comprise genomic modifications of β2m.

89. The method of claim 54 or 55, wherein the genomic modifications do not comprise genomic modifications of one or more of β2m, RFX5, NLRC5, CIITA, and TAP2.

90. The method of any one of claims 59-83, wherein the expression of β2m is not functionally impaired or reduced in the engineered immune cell.

91. The method of any one of claims 59-83, wherein the genomic modifications do not comprise genomic modifications of β2m.

92. The method of any one of claims 59-83, wherein the genomic modifications do not comprise genomic modifications of one or more of β2m, RFX5, NLRC5, CIITA, and TAP2.

93. The engineered immune cell of claim 68 or 69, which expresses the CD70 binding protein.

94. The engineered immune cell of any one of claims 11-30, which further comprises a polynucleotide sequence encoding a CD70 binding protein.

95. The engineered cell of claim 94, which expresses the CD70 binding protein.

96. The engineered cell of any one of claims 1-30 and 84-85, or the population of any one of claims 31-53 and 86-87, or the method of any one of claims 54-55, 57-83, and 88-92, wherein the CD70 binding protein comprises a CD70 binding domain and a transmembrane domain.

97. The engineered cell, population, or method of claim 96, wherein the CD70 binding domain comprises a CD70 antibody, or a receptor for CD70 or a CD70 binding fragment thereof.

98. The engineered cell, population, or method of claim 96 or 97, wherein the CD70 binding domain comprises an anti-CD70 antibody, optionally the anti-CD70 antibody is a scFv.

99. The engineered cell, population, or method of any one of claims 96-98, wherein the CD70 binding protein further comprises a hinge domain, optionally the hinge domain comprises a CD8 hinge.

100. The engineered cell, population, or method of any one of claims 96-99, wherein the CD70 binding protein further comprises one or more intracellular signaling domains selected from the group consisting of a CD3z signaling domain, a CD3d signaling domain, a CD3g signaling domain, a CD3e signaling domain, a CD28 signaling domain, a CD2 signaling domain, an OX40 signaling domain, and a 4-1BB signaling domain, or a variant thereof.

101. The engineered cell, population, or method of any one of claims 96-100, wherein the CD70 binding protein comprises a CD3z or a CD3g signaling domain and does not comprise a costimulatory domain.

102. The engineered cell, population, or method of any one of claims 96-101, wherein the CD70 binding protein comprises a 4-1BB signaling domain and does not comprise a CD3z signaling domain.

103. The engineered cell, population, or method of any one of claims 96-98, wherein the CD70 binding protein comprises a 4-1BB signaling domain and a CD3z signaling domain.

102. The engineered immune cell of any one of claims 109-101, wherein the one or more intracellular domain comprises the amino acid sequence of one or more of SEQ ID NOs: 1, 7-14, 17-70, or 89-90.

103. The engineered cell, population, or method of any one of claims 96-102, wherein the CD70 binding protein does not comprise an intracellular signaling domain.


SEQUENCE LISTING (105 sequences)

SEQ ID NO: 1 - protein, 225 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 21 aa, synthetic construct
MALPVTALLLPLALLLHAARP

SEQ ID NO: 3 - protein, 16 aa, synthetic construct
GLAVSTISSFFPPGYQ

SEQ ID NO: 4 - protein, 45 aa, synthetic construct
TTTPAPRPPTPAPTIASQPLSLRPEACRPAAGGAVHTRGLDFACD

SEQ ID NO: 5 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, synthetic construct
IYIWAPLAGTCGVLLLSLVITLYC

SEQ ID NO: 7 - protein, 42 aa, synthetic construct
KRGRKKLLYIFKQPFMRPVQTTQEEDGCSCRFPEEEEGGCEL

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 52 aa, synthetic construct
FFIPLLVVILFAVDTGLFISTQQQVTFLLKIKRTRKGFRLLNPHPKPNPKNN

SEQ ID NO: 10 - protein, 215 aa, synthetic construct
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 11 - protein, 45 aa, synthetic construct
GHETGRLSGAADTQALLRNDQVYQPLRDRDDAQYSHLGGNWARNK

SEQ ID NO: 12 - protein, 41 aa, synthetic construct
RSKRSRGGHSDYMNMTPRRPGPTRKHYQPYAPPRDFAAYRS

SEQ ID NO: 13 - protein, 18 aa, synthetic construct
MIPAVVLLLLLLVEQAAA

SEQ ID NO: 14 - protein, 43 aa, synthetic construct
LGEPQLCYILDAILFLYGIVLTLLYCRLKIQVRKAAITSYEKS

SEQ ID NO: 15 - protein, 22 aa, synthetic construct
GSGATNFSLLKQAGDVEENPGP

SEQ ID NO: 16 - protein, 21 aa, synthetic construct
GSGEGRGSLLTCGDVEENPGP

SEQ ID NO: 17 - protein, 55 aa, synthetic construct
KNRKAKAKPVTRGAGAGGRQRGQNKERPPPVPNPDYEPIRKGQRDLYSGLNQRRI

SEQ ID NO: 18 - protein, 45 aa, synthetic construct
GQDGVRQSRASDKQTLLPNDQLYQPLKDREDDQYSHLQGNQLRRN

SEQ ID NO: 19 - protein, 29 aa, synthetic construct
APAYQQGQNQLYNELNLGRREEYDVLDKR

SEQ ID NO: 20 - protein, 28 aa, synthetic construct
PRRKNPQEGLYNELQKDKMAEAYSEIGM

SEQ ID NO: 21 - protein, 29 aa, synthetic construct
ERRRGKGHDGLYQGLSTATKDTYDALHMQ

SEQ ID NO: 22 - protein, 29 aa, synthetic construct
DTQALLRNDQVYQPLRDRDDAQYSHLGGN

SEQ ID NO: 23 - protein, 28 aa, synthetic construct
ERPPPVPNPDYEPIRKGQRDLYSGLNQR

SEQ ID NO: 24 - protein, 29 aa, synthetic construct
DKQTLLPNDQLYQPLKDREDDQYSHLQGN

SEQ ID NO: 25 - protein, 112 aa, synthetic construct
RVKFSRSADDTQALLRNDQVYQPLRDRDDAQYSHLGGNRGRDPEMGGKPRRKNPQEGLYNELQKDKMAEAYSEIGMKGERRRGKGHDGLYQGLSTATKDTYDALHMQALPPR

SEQ ID NO: 26 - protein, 111 aa, synthetic construct
RVKFSRSADERPPPVPNPDYEPIRKGQRDLYSGLNQRRGRDPEMGGKPRRKNPQEGLYNELQKDKMAEAYSEIGMKGERRRGKGHDGLYQGLSTATKDTYDALHMQALPPR

SEQ ID NO: 27 - protein, 112 aa, synthetic construct
RVKFSRSADDKQTLLPNDQLYQPLKDREDDQYSHLQGNRGRDPEMGGKPRRKNPQEGLYNELQKDKMAEAYSEIGMKGERRRGKGHDGLYQGLSTATKDTYDALHMQALPPR

SEQ ID NO: 28 - protein, 112 aa, synthetic construct
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 29 - protein, 225 aa, synthetic construct
RVKFSRSADAPAYQQGQNQLYNELNLGRREEYDVLDKRRGRDPEMGGKDTQALLRNDQVYQPLRDRDDAQYSHLGGNKGPRRKNPQEGLYNELQKDKMAEAYSEIGMALPPRGRVKFSRSADERPPPVPNPDYEPIRKGQRDLYSGLNQRRGRDPEMGGKERRRGKGHDGLYQGLSTATKDTYDALHMQKGDKQTLLPNDQLYQPLKDREDDQYSHLQGNALPPR

SEQ ID NO: 30 - protein, 225 aa, synthetic construct
RVKFSRSADDTQALLRNDQVYQPLRDRDDAQYSHLGGNRGRDPEMGGKAPAYQQGQNQLYNELNLGRREEYDVLDKRKGERPPPVPNPDYEPIRKGQRDLYSGLNQRALPPRGRVKFSRSADPRRKNPQEGLYNELQKDKMAEAYSEIGMRGRDPEMGGKDKQTLLPNDQLYQPLKDREDDQYSHLQGNKGERRRGKGHDGLYQGLSTATKDTYDALHMQALPPR

SEQ ID NO: 31 - protein, 225 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 32 - protein, 225 aa, synthetic construct
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 112 aa, synthetic construct
RVKFSRSADAPAYQQGQNQLYAELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 112 aa, synthetic construct
RVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 35 - protein, 43 aa, synthetic construct
RVKFSRSADAPAYQQGQNQLYAELNLGRREEYDVLDKRALPPR

SEQ ID NO: 36 - protein, 225 aa, synthetic construct
RVKFSRSADAPAYQQGQNQLYAELNLGRREEYDVLDKRRGRDPEMGGKDTQALLRNDQVYAPLRDRDDAQYSHLGGNKGPRRKNPQEGLYAELQKDKMAEAYSEIGMALPPRGRVKFSRSADERPPPVPNPDYAPIRKGQRDLYSGLNQRRGRDPEMGGKERRRGKGHDGLYAGLSTATKDTYDALHMQKGDKQTLLPNDQLYAPLKDREDDQYSHLQGNALPPR

SEQ ID NO: 37 - protein, 225 aa, synthetic construct
RVKFSRSADDTQALLRNDQVYAPLRDRDDAQYSHLGGNRGRDPEMGGKAPAYQQGQNQLYAELNLGRREEYDVLDKRKGERPPPVPNPDYAPIRKGQRDLYSGLNQRALPPRGRVKFSRSADPRRKNPQEGLYAELQKDKMAEAYSEIGMRGRDPEMGGKDKQTLLPNDQLYAPLKDREDDQYSHLQGNKGERRRGKGHDGLYAGLSTATKDTYDALHMQALPPR

SEQ ID NO: 38 - protein, 29 aa, synthetic construct
APAYQQGQNQLYAELNLGRREEYDVLDKR

SEQ ID NO: 39 - protein, 28 aa, synthetic construct
PRRKNPQEGLYAELQKDKMAEAYSEIGM

SEQ ID NO: 40 - protein, 29 aa, synthetic construct
ERRRGKGHDGLYAGLSTATKDTYDALHMQ

SEQ ID NO: 41 - protein, 29 aa, synthetic construct
DTQALLRNDQVYAPLRDRDDAQYSHLGGN

SEQ ID NO: 42 - protein, 28 aa, synthetic construct
ERPPPVPNPDYAPIRKGQRDLYSGLNQR

SEQ ID NO: 43 - protein, 29 aa, synthetic construct
DKQTLLPNDQLYAPLKDREDDQYSHLQGN

SEQ ID NO: 44 - protein, 29 aa, synthetic construct
APAYQQGQNQLYAELNLGRREEYDVLDKR

SEQ ID NO: 45 - protein, 28 aa, synthetic construct
PRRKNPQEGLYAELQKDKMAEAYSEIGM

SEQ ID NO: 46 - protein, 29 aa, synthetic construct
ERRRGKGHDGLYAGLSTATKDTYDALHMQ

SEQ ID NO: 47 - protein, 29 aa, synthetic construct
DTQALLRNDQVYAPLRDRDDAQYSHLGGN

SEQ ID NO: 48 - protein, 28 aa, synthetic construct
ERPPPVPNPDYAPIRKGQRDLYSGLNQR

SEQ ID NO: 49 - protein, 29 aa, synthetic construct
DKQTLLPNDQLYAPLKDREDDQYSHLQGN

SEQ ID NO: 50 - protein, 29 aa, synthetic construct
APAYQQGQNQLYAELNLGRREEYDVLDKR

SEQ ID NO: 51 - protein, 28 aa, synthetic construct
PRRKNPQEGLYAELQKDKMAEAYSEIGM

SEQ ID NO: 52 - protein, 29 aa, synthetic construct
ERRRGKGHDGLYAGLSTATKDTYDALHMQ

SEQ ID NO: 53 - protein, 29 aa, synthetic construct
DTQALLRNDQVYAPLRDRDDAQYSHLGGN

SEQ ID NO: 54 - protein, 28 aa, synthetic construct
ERPPPVPNPDYAPIRKGQRDLYSGLNQR

SEQ ID NO: 55 - protein, 29 aa, synthetic construct
DKQTLLPNDQLYAPLKDREDDQYSHLQGN

SEQ ID NO: 56 - protein, 29 aa, synthetic construct
APAYQQGQNQLYAELNLGRREEYDVLDKR

SEQ ID NO: 57 - protein, 41 aa, synthetic construct
RSKRSRLLHSDYMNMTPRRPGPTRKHYQPYAPPRDFAAYRS

SEQ ID NO: 58 - protein, 41 aa, synthetic construct
RSKRSRLLHSDYMFMTPRRPGPTRKHYQPYAPPRDFAAYRS

SEQ ID NO: 59 - protein, 41 aa, synthetic construct
RSKRSRLLHSDYMNMTPRRPGPTRKHYQAYAAPRDFAAYRS

SEQ ID NO: 60 - protein, 41 aa, synthetic construct
RSKRSRLLHSDFMNMTARRAGPTRKHYQPYAPPRDFAAYRS

SEQ ID NO: 61 - protein, 116 aa, synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 62 - protein, 85 aa, synthetic construct
KRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 63 - protein, 42 aa, synthetic construct
ALYLLRRDQRLPPDAHKPPGGGSFRTPIQEEQADAHSTLAKI

SEQ ID NO: 64 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 65 - protein, 112 aa, synthetic construct
RVKFSRSADAPAYQQGQNQLFNELNLGRREEFDVLDKRRGRDPEMGGKPRRKNPQEGLFNELQKDKMAEAFSEIGMKGERRRGKGHDGLYQGLSTATKDTYDALHMQALPPR

SEQ ID NO: 66 - protein, 48 aa, synthetic construct
QRRKYRSNKGESPVEPAEPCHYSCPREEEGSTIPIQEDYRKPEPACSP

SEQ ID NO: 67 - protein, 112 aa, synthetic construct
RVKFSRSADAPAYQQGQNQLFNELNLGRREEFDVLDKRRGRDPEMGGKPRRKNPQEGLYNELQKDKMAEAYSEIGMKGERRRGKGHDGLFQGLSTATKDTFDALHMQALPPR

SEQ ID NO: 68 - protein, 48 aa, synthetic construct
RVKFSRSADERRRGKGHDGLYQGLSTATKDTYDALHMQRGRDPEMGGK

SEQ ID NO: 69 - protein, 48 aa, synthetic construct
RVKFSRSADAPAYQQGQNQLYNELNLGRREEYDVLDKRRGRDPEMGGK

SEQ ID NO: 70 - protein, 37 aa, synthetic construct
RVKFSRSADGVYNALQKDKMAEAYSEIRGRDPEMGGK

SEQ ID NO: 71 - protein, 9 aa, synthetic construct
CPYSNPSLC

SEQ ID NO: 72 - protein, 15 aa, synthetic construct
GGGGSGGGGSGGGGS

SEQ ID NO: 73 - protein, 5 aa, synthetic construct
GGGGS

SEQ ID NO: 74 - protein, 20 aa, synthetic construct
GGGGSGGGGSGGGGSGGGGS

SEQ ID NO: 75 - protein, 18 aa, synthetic construct
GSTSGSGKPGSGEGSTKG

SEQ ID NO: 76 - protein, 16 aa, synthetic construct
ELPTQGTFSNVSTNVS

SEQ ID NO: 77 - protein, 24 aa, synthetic construct
ELPTQGTFSNVSTNVSPAKPTTTA

SEQ ID NO: 78 - protein, 55 aa, synthetic construct
GSGGGGSCPYSNPSLCSGGGGSELPTQGTFSNVSTNVSPAKPTTTACPYSNPSLC

SEQ ID NO: 79 - protein, 62 aa, synthetic construct
GSGGGGSELPTQGTFSNVSTNVSPAKPTTTAGSGGGGSELPTQGTFSNVSTNVSPAKPTTTA

SEQ ID NO: 80 - protein, 39 aa, synthetic construct
IEVMYPPPYLDNEKSNGTIIHVKGKHLCPSPLFPGPSKP

SEQ ID NO: 81 - protein, 27 aa, synthetic construct
FWVLVVVGGVLACYSLLVTVAFIIFWV

SEQ ID NO: 82 - protein, 250 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 83 - protein, 245 aa, synthetic construct
QVQLQESGPGLVQPSETLSLTCTVSGGSISYYYWSWIRQPPGKGLEWIGNINYMGNTIYNPSLKSRVTISVDTSKDQFSLKLTSVSAADTAVYYCVRAEGSLDAFDFWGQGTLVAVSLGGGGSGGGGSGGGGSGGGGSDIQMTQSPSTLSASVGDRVTITCRASQSISSWLAWYQQKPGKAPKVLIYKASNLESGVPSRFSGSGSGTEFTLTISSLQPDDFATYYCQQYNSYSCTFGQGTKLEIK

SEQ ID NO: 84 - protein, 193 aa, synthetic construct
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 85 - protein, 245 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 86 - protein, 447 aa, synthetic construct
MALPVTALLLPLALLLHAARPQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 107 aa, synthetic construct
DIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 88 - protein, 123 aa, synthetic construct
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSS

SEQ ID NO: 89 - protein, 40 aa, synthetic construct
GRKKLLYIFKQPFMRPVQTTQEEDGCSCRFPEEEEGGCEL

SEQ ID NO: 90 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - RNA, 20 nt, synthetic construct
  source 1..20: mol_type = other RNA
gctggtacacggcagggtca

SEQ ID NO: 92 - RNA, 20 nt, synthetic construct
  source 1..20: mol_type = other RNA
tcacgtcatccagcagagaa

SEQ ID NO: 93 - RNA, 20 nt, synthetic construct
  source 1..20: mol_type = other RNA
cagctctgcaaggctctggg

SEQ ID NO: 94 - RNA, 20 nt, synthetic construct
  source 1..20: mol_type = other RNA
agtacatgtatgcctatagg

SEQ ID NO: 95 - RNA, 20 nt, synthetic construct
  source 1..20: mol_type = other RNA
aaggcacattgcttggtaca

SEQ ID NO: 96 - RNA, 20 nt, synthetic construct
  source 1..20: mol_type = other RNA
tcacttggtacatatgaccg

SEQ ID NO: 97 - RNA, 20 nt, synthetic construct
  source 1..20: mol_type = other RNA
tgagggggacacagatgtct

SEQ ID NO: 98 - RNA, 20 nt, synthetic construct
  source 1..20: mol_type = other RNA
gtggcacactgtgagctgcc

SEQ ID NO: 99 - RNA, 20 nt, synthetic construct
  source 1..20: mol_type = other RNA
ttgttgacgaggttgtcacc

SEQ ID NO: 100 - RNA, 20 nt, synthetic construct
  source 1..20: mol_type = other RNA
gctcgtctggcttgttgacg

SEQ ID NO: 101 - RNA, 20 nt, synthetic construct
  source 1..20: mol_type = other RNA
gtcaacaagccagacgagcg

SEQ ID NO: 102 - RNA, 20 nt, synthetic construct
  source 1..20: mol_type = other RNA
tcttagcagcatggaggcgc

SEQ ID NO: 103 - RNA, 20 nt, synthetic construct
  source 1..20: mol_type = other RNA
cttagcagcatggaggcgca

SEQ ID NO: 104 - RNA, 20 nt, synthetic construct
  source 1..20: mol_type = other RNA
catggaggcgcagggtgtag

SEQ ID NO: 105 - RNA, 20 nt, synthetic construct
  source 1..20: mol_type = other RNA
cgcgtccaccagcagcaggg